SEQUENCE LISTING (35 sequences)

SEQ ID NO: 1 - protein, 34 aa, Artificial Sequence
  Artificial protein based on human PYY
IKPEAPGEDASPEELNRYYASLRHYLNLVTRQRY

SEQ ID NO: 2 - protein, 34 aa, Artificial Sequence; identical to SEQ ID NO: 1
  artificial protein based on human PYY

SEQ ID NO: 3 - protein, 34 aa, Artificial Sequence; identical to SEQ ID NO: 1
  artificial protein based on human PYY

SEQ ID NO: 4 - protein, 34 aa, Artificial Sequence; identical to SEQ ID NO: 1
  artificial protein based on human PYY

SEQ ID NO: 5 - protein, 34 aa, Artificial Sequence
  artificial protein based on human PYY
IKPEAPGEDASPEELNRYYASLHHYLNLVTRQRY

SEQ ID NO: 6 - protein, 34 aa, Artificial Sequence
  artificial protein based on human PYY
IKPEAPGEDASPEELNRYYASLXHYLNLVTRQRY

SEQ ID NO: 7 - protein, 34 aa, Artificial Sequence
  artificial protein based on human PYY
IKPEAPGEDASPEELNRYYASLYHYLNLVTRQRY

SEQ ID NO: 8 - protein, 34 aa, Artificial Sequence
  artificial protein based on human PYY
IKPEAPGEDASPEELNRAYASLRHYLNLVTRQRY

SEQ ID NO: 9 - protein, 34 aa, Artificial Sequence; identical to SEQ ID NO: 1
  artificial protein based on human PYY

SEQ ID NO: 10 - protein, 34 aa, Artificial Sequence
  artificial protein based on human PYY
IKPEAPGEDASPEELNRYYASLRHALNLVTRQRY

SEQ ID NO: 11 - protein, 34 aa, Artificial Sequence
  artificial protein based on human PYY
IKPEAPGEDASPEELNRYYASLRHYLNLVTRQRF

SEQ ID NO: 12 - protein, 34 aa, Artificial Sequence; identical to SEQ ID NO: 1
  artificial protein based on human PYY

SEQ ID NO: 13 - protein, 34 aa, Artificial Sequence; identical to SEQ ID NO: 1
  artificial protein based on human PYY

SEQ ID NO: 14 - protein, 34 aa, Artificial Sequence; identical to SEQ ID NO: 1
  artificial protein based on human PYY

SEQ ID NO: 15 - protein, 34 aa, Artificial Sequence; identical to SEQ ID NO: 1
  artificial protein based on human PYY

SEQ ID NO: 16 - protein, 34 aa, Artificial Sequence
  artificial protein based on human PYY
IKPEAPGEDASPEELNRYYASLRHYLNLVTRQXY

SEQ ID NO: 17 - protein, 34 aa, Artificial Sequence; identical to SEQ ID NO: 16
  artificial protein based on human PYY

SEQ ID NO: 18 - protein, 34 aa, Artificial Sequence; identical to SEQ ID NO: 1
  artificial protein based on human PYY

SEQ ID NO: 19 - protein, 34 aa, Artificial Sequence; identical to SEQ ID NO: 1
  artificial protein based on human PYY

SEQ ID NO: 20 - protein, 34 aa, Artificial Sequence; identical to SEQ ID NO: 16
  artificial protein based on human PYY

SEQ ID NO: 21 - protein, 34 aa, Artificial Sequence; identical to SEQ ID NO: 16
  artificial protein based on human PYY

SEQ ID NO: 22 - protein, 34 aa, Artificial Sequence; identical to SEQ ID NO: 1
  artificial protein based on human PYY

SEQ ID NO: 23 - protein, 34 aa, Artificial Sequence; identical to SEQ ID NO: 1
  artificial protein based on human PYY

SEQ ID NO: 24 - protein, 34 aa, Artificial Sequence; identical to SEQ ID NO: 1
  artificial protein based on human PYY

SEQ ID NO: 25 - protein, 34 aa, Artificial Sequence; identical to SEQ ID NO: 1
  Articificial protein based on human PYY

SEQ ID NO: 26 - protein, 34 aa, Artificial Sequence; identical to SEQ ID NO: 1
  Articificial protein based on human PYY

SEQ ID NO: 27 - protein, 34 aa, Artificial Sequence; identical to SEQ ID NO: 1
  Artificial protein based on human PYY

SEQ ID NO: 28 - protein, 34 aa, Artificial Sequence; identical to SEQ ID NO: 1
  Artificial protein based on human PYY

SEQ ID NO: 29 - protein, 34 aa, Artificial Sequence
  Artificial protein based on human PYY
IKPEAPGEDAKPEELNRYYASLRHYLNLVTRQRY

SEQ ID NO: 30 - protein, 34 aa, Artificial Sequence; identical to SEQ ID NO: 1
  Artificial protein based on human PYY

SEQ ID NO: 31 - protein, 34 aa, Artificial Sequence; identical to SEQ ID NO: 1
  Artificial protein based on human PYY

SEQ ID NO: 32 - protein, 34 aa, Artificial Sequence; identical to SEQ ID NO: 1
  Artificial protein based on human PYY

SEQ ID NO: 33 - protein, 34 aa, Artificial Sequence; identical to SEQ ID NO: 1
  Artificial protein based on human PYY

SEQ ID NO: 34 - protein, 34 aa, Artificial Sequence; identical to SEQ ID NO: 1
  Artificial protein based on human PYY

SEQ ID NO: 35 - protein, 34 aa, Artificial Sequence
  Artificial protein based on human PYY
IKPEAPGKDASPEELNRYYASLRHYLNLVTRQRY

(19) United States
(12) Patent Application Publication
(10) Pub. No.: US 2013/0040877 A1
(43) Pub. Date: Feb. 14, 2013

(54) LONG-ACTING Y2 RECEPTOR AGONISTS

(71) Applicants: Jacob Kofoed, Vaerlose (DK); Jorgen Olsen, Vaerlose (DK); Soeren Ostergaard, Broenshoej (DK); Rasmus Jorgensen, Maaloev (DK)

(72) Inventors: Jacob Kofoed, Vaerlose (DK); Jorgen Olsen, Vaerlose (DK); Soeren Ostergaard, Broenshoej (DK); Rasmus Jorgensen, Maaloev (DK)

(73) Assignee: Novo Nordisk A/S, Bagsvaerd (DK)

(21) Appl. No.: 13/496,266

(22) PCT Filed: Sep. 17, 2010
(86) PCT No.: PCT/EP2010/063707

§ 371 Date: Apr. 17, 2012

Related U.S. Application Data
(60) Provisional application No. 61/244,511, filed on Sep. 22, 2009.
(60) Provisional application No. 61/261,769, filed on Nov. 17, 2009.

(30) Foreign Application Priority Data
Sep. 18, 2009 (EP) 09170670.5
Nov. 13, 2009 (EP) 09175893.8

(51) Int. Cl. A61K 38/16; A61P 3/10; C07K 14/00

(52) U.S. Cl. 514/6.9; 530/324

(57) ABSTRACT

The present invention relates to PYY analogues or derivatives thereof comprising at least one alteration selected from the group consisting of substitutions, insertions, deletions and modifications and optionally a serum albumin binding side chain comprising an alkyl chain with at least 14 carbon atoms. Moreover, the invention relates to compositions hereof and methods of treatment of conditions responsive to Y2 receptor modulation.

### FIELD OF THE INVENTION

This invention relates to the field of therapeutic peptides, i.e. to new protracted peptide analogues such as Peptide YY (PYY) analogues as well as derivatives thereof, wherein said analogue or derivative comprises at least one alteration in the peptide selected from the group consisting of substitutions, deletions, insertion and modifications which stabilize said peptide analogues against proteolytic breakdown.

### BACKGROUND OF THE INVENTION

Peptide Tyrosine-Tyrosine (PYY) is released during a meal from L-cells in the distal small intestine and the colon. PYY is known to have peripheral effects in the GI-tract and also act centrally as a satiety signal. PYY is released as PYY(1-36) but is cleaved to PYY(3-36) which constitutes approximately 50% of the circulating PYY. The enzyme primarily responsible for the degradation into PYY(3-36) in vivo is dipeptidyl peptidase IV (DPP IV). PYY(3-36) is rapidly eliminated by proteases and other clearance mechanisms. The half life of PYY(3-36) has been reported to be <30 minutes in pigs. Thus, PYY display suboptimal pharmacokinetic properties and has to be administered at least once-daily in order to exert therapeutic effect.

Whereas hPYY(1-36) activates Y1, Y2 and Y5 receptors with very little selectivity the DPP IV processed hPYY(3-36) display increased selectivity for the Y2 receptor albeit some Y1 and Y5 affinity is retained. Y2 receptor activation is known to decrease appetite and food-intake whereas Y1 and Y5 receptor activation leads to an increase in appetite and food-intake. Furthermore, Y1 and Y5 receptor activation can an increase in blood pressure.

PYY signals through the Y2 receptor and the determinants for binding to the Y2 receptor are mainly located in the C-terminal part of the peptide. Accordingly, a change in the C-terminal part of PYY involves a risk of loss of therapeutic effect.

PYY has been suggested for use in the treatment of obesity and associated diseases based on the demonstrated effects of certain of these peptides in animal models and in man and on the fact that obese people have low basal levels of PYY as well as lower meal responses of this peptide. Furthermore, Y2 agonists have been demonstrated to have anti-secretory and pro-absorptive effects in the gastro-intestinal (GI) tract. The potential use of Y2 agonists in the treatment of a number of gastro-intestinal disorders has been suggested.

For treatment of conditions responsive to Y2 receptor modulation such as obesity and intestinal hyper-secretion it would be desirable to use protracted Y2 receptor selective agonists, such as protracted PYY. The relatively short half life of PYY(3-36) limits therapeutic use of this peptide as a steady exposure level would require frequent dosing which would be highly inconvenient for the patients.

Bioactive peptides need to be inactivated after stimulation of their respective receptors. Inactivation routes for circulating peptides include degradation by cell-surface or soluble plasma peptidases as well as other types of clearance mechanisms. Said degradation by proteases is accomplished by a panel of proteases, including both highly specialized peptidases which regulate receptor selectivity of peptides as well as broad specificity peptidases responsible for complete peptide inactivation. Among said clearance mechanisms, the renal clearance is the most important for the removal of PYY albeit also liver clearances in some instances play an important role, while for most peptides and proteins the clearance by perspiration and respiration does not exist. Accordingly, it is desirable to prolong the in vivo half life of PYY. In order to prolong the half life of PYY the peptide can be attached to a larger protein such as albumin.

For the treatment of conditions, such as obesity, responsive to Y receptor modulation it would be attractive to use PYY analogues, which activate the Y receptor subtype Y2 and importantly also display protracted pharmacokinetic properties and as such can be used in a dosing regime with lower frequency of administration than the human PYY, such as human PYY(3-36).

In humans nausea and vomiting have been described as dose limiting adverse events for treatment with Y2 receptor agonists. The therapeutic window, where a positive treatment effect of administration can be observed without any adverse events, has been described to be narrow for Y2 receptor agonists. Furthermore, animal studies suggest that chronic exposure is necessary to obtain optimal therapeutic effect. Due to the narrow therapeutic window, which is clinically described for PYY treatment, it is desirable to obtain the PYY analogue or derivative thereof with improved pharmacokinetic properties compared to human PYY(1-36), human PYY(3-36) or human PYY(3-36) with a serum albumin binding side chain identical to that of said derivative. Said improved pharmacokinetic properties would allow a better titration within the therapeutic window and/or enable avoiding multiple daily administrations.

### SUMMARY OF THE INVENTION

In one embodiment the invention relates to the PYY analogue or a derivative thereof with improved stability against C-terminal proteolytic breakdown which breakdown decreases functionality of said analogue or derivative as compared to human PYY(1-36) or human PYY(3-36), wherein said functionality is determined by Assay (IV), Assay (I) or Assay (V) as described herein.

In one embodiment the invention relates to a PYY analogue or a derivative thereof comprising
i. at least one serum albumin binding side chain comprising an alkyl chain with at least 14 carbon atoms, wherein said alkyl chain optionally further comprises a distal carboxylic acid or a distal tetrazole group; and
ii. at least one amino acid residue substituted into a proteinogenic or non-proteinogenic amino acid residue selected from the group consisting of

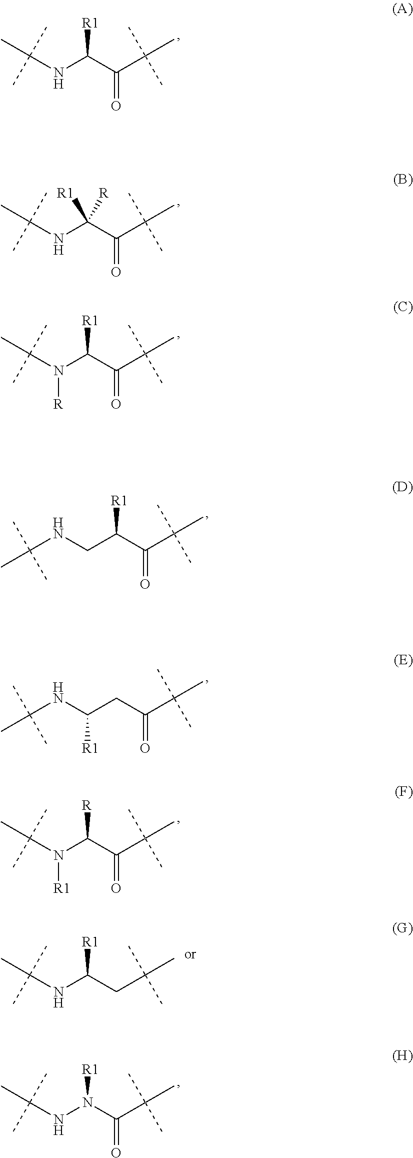

wherein R1 is side a chain of an amino acid and R is H or C1-C12 alkyl.

In one embodiment the invention relates to the PYY analogue or a derivative as defined herein for use in the treatment of a condition responsive to Y receptor modulation, such as obesity, obesity-related diseases, e.g., reduction of food intake, or diabetes. In one embodiment the invention relates to a composition comprising the PYY analogue or a derivative thereof as defined herein and at least one pharmaceutical excipient.

### DESCRIPTION OF THE DRAWINGS

FIG. 1 shows the effect of peptide SEQ ID NO: 3 on food intake in lean fasted-re-fed C57BL/6 mice. The mice were administrated s.c. doses of the peptides in 0.1, 0.3 and 1.0 umol/kg as well as vehicle alone 30 min before food return and cumulative food intake was measured over 48 hr.

### DESCRIPTION OF THE INVENTION

In one embodiment the present invention relates to PYY analogues or derivatives thereof that contain at least one alteration selected from the group consisting of substitutions, deletions and modifications in the peptide backbone which stabilize said analogue or derivative against proteolytic breakdown in vivo. In one embodiment said analogues or derivatives are stabilized against C-terminal proteolytic breakdown.

As stated above, PYY(1-36) is rapidly degraded to the 3-36 form by DPP IV. Further N-terminal degradation is prevented by the polyproline helix. The present inventors have found by in vitro and in vivo investigations that PYY(3-36) is cleaved in the C-terminal, resulting in complete loss of Y2 receptor potency. Therefore, PYY(3-36) (as well as PYY(1-36)) displays suboptimal pharmacokinetic profile with a short half life and would need to be administered frequently, such as at least once-daily or at least twice-daily, in order to obtain a therapeutic effect.

In one embodiment this invention relates to the PYY analogue or derivative thereof which has protracted pharmacokinetic properties. In one embodiment this invention relates to the PYY analogue or derivative thereof which is an agonist of the Y2 receptor and has protracted pharmacokinetic properties. The term “agonist” means any compound that activates the target receptor and elicits at least one of the in vivo or in vitro effects elicited by the endogenous agonist for said receptor. In one embodiment the derivative of the PYY analogue according to the invention show surprisingly good protracted pharmacokinetic properties.

“Protracted properties” of a peptide is prolonged duration of action of the peptide which results in a dosing regime with lower frequency, e.g., once-daily, once-weekly or less than once-weekly. The protracted properties of the PYY analogue or derivative thereof according to the invention could manifest as prolonged half life in plasma or prolonged biological activity compared to human PYY(1-36), human PYY(3-36) or human PYY(3-36) with a serum albumin binding side chain identical to that of said analogue or derivative. In one embodiment the protraction of the PYY analogue or derivative thereof is determined by monitoring the concentration thereof in plasma after administration to animals, such as healthy pigs, using methods as described herein, such as Assay (V) described herein, PK i.v. mini-pig. In one embodiment the protraction of the PYY analogue or derivative thereof is determined by monitoring the duration of effect of said analogue or derivative in at least one biological assay, such as Assay (V), Assay (VII), Assay (VI), Assay (VIII) or Assay (IX) described herein.

The terms “human PYY” and “hPYY” are intended to mean PYY(1-36). In one embodiment human PYY is as defined by SEQ ID NO: 1 and with addition of the N-terminal amino acids Tyr-Pro in position 1 and 2. In one embodiment human PYY is PYY(3-36) as defined by SEQ ID NO: 1. In one embodiment a combination of at least one of the features mentioned herein is achieved.

The beneficial effects of PYY(3-36) is believed to be mediated through the Y2 receptor while activation of the Y1 and Y5 receptors can lead to adverse effects or abolish the therapeutic effect. Thus, it is desirable to develop PYY analogues or derivatives thereof with increased selectivity for the Y2 receptor relative to the Y1 and/or Y5 receptors. An increase in Y2 receptor selectivity is intended to mean a relative decrease in Y1 and/or Y5 receptor potency compared to Y2 receptor potency or a relative increase in Y2 receptor potency compared to Y1 and/or Y5 receptor potency. Thus a PYY(3-36) analogue or derivative thereof which, compared to hPYY(3-36), display a relatively larger decrease in potency on the Y1 and/or Y5 receptor than on the Y2 receptor as determined by Assay (II) and/or (III) and (I), respectively, has an increased Y2 receptor selectivity. In one embodiment the PYY analogues or derivatives thereof have increased selectivity for the Y2 receptor relative to the Y1 and/or Y5 receptor compared to hPYY(3-36). In one embodiment the PYY analogue or derivative thereof has increased selectivity for the Y2 receptor relative to the Y1 receptor, i.e. a higher Y1/Y2 receptor potency ratio, compared to hPYY(3-36). In one embodiment the PYY analogue or derivative thereof has increased selectivity for the Y2 receptor relative to the Y5 receptor, i.e. a higher Y5/Y2 receptor potency ratio, compared to hPYY(3-36). In one embodiment the PYY analogues or derivatives thereof have increased selectivity for the Y2 receptor relative to the Y1 and/or Y5 receptor and are stabilized against C-terminal degradation. In one embodiment the PYY analogues or derivatives thereof have increased selectivity for the Y2 receptor relative to the Y1 and/or the Y5 receptor and have protracted pharmacokinetic properties, such as a longer half-life.

### PYY Analogue

In one embodiment the PYY analogue or derivative obtains protracted pharmacokinetic properties by incorporating alterations selected from the group consisting of substitutions, deletions and modifications into the PYY peptide, wherein the PYY peptide may be human PYY(1-36) or human PYY(3-36). Prevention of proteolysis of the PYY analogue or derivative thereof is possible by incorporating alterations selected from the group consisting of substitutions, deletions and modifications of amino acid residues at or close to the cleavage site of interest with residues not accepted by the active site in the cleaving protease.

A concept that could be employed for obtaining PYY analogues or derivatives thereof is based on the introduction of the imino acid proline in close proximity to the cleavage site disrupting peptide conformation and reducing peptide backbone flexibility resulting in stabilization of the peptide, however, this substitution bears the risk of pertubing the therapeutic effect of said analogue or derivative. In one embodiment the PYY analogue or derivative thereof comprises at least one N-methyl amino acid analogue of an amino acid residue of interest. N-methyl amino acid analogues have the potential to preserve the therapeutic effect of the PYY analogue or derivative thereof since the amino acid side chain is not altered and to enhance the proteolytic stability of the PYY analogue or derivative thereof due to the structural similarity with proline around the peptide bond. Other proteolysis stabilizing concepts include the use of hydrocarbon stabling or alterations to the peptide bond reduced peptide bonds.

The term “analogue” as used herein referring to a peptide means a peptide wherein at least one amino acid residue of the peptide has been substituted with another amino acid residue and/or wherein at least one amino acid residue has been deleted from the peptide and/or wherein at least one amino acid residue has been added to the peptide and/or wherein at least one amino acid residue of the peptide has been modified.

Such addition or deletion of amino acid residues can take place at the N-terminal of the peptide and/or at the C-terminal of the peptide. A simple nomenclature is used to describe the peptides according to the invention, e.g., [N-methyl Arg35] hPYY(3-36) designates an analogue of the human PYY wherein the naturally occurring arginine in position 35 has been substituted with N-methylated arginine and the naturally occurring tyrosine and proline in position 1 and 2, respectively, have been deleted.

In one embodiment references herein to positions in a peptide or the PYY analogue or derivative thereof refers to positions in human PYY(1-36), such as in human PYY(3-36). In one embodiment the N-terminal position in PYY(3-36) is referred to as position 3. In one embodiment the PYY analogue or derivative thereof may be derived from vertebrates, such as a mammal, including human, mouse, sheep, goat, cow, or horse.

The term “peptide” as used herein means a compound composed of at least five constituent amino acids connected by peptide bonds. In one embodiment the N-terminus of the peptide is an amino group and/or said C-terminus is a carboxylic acid group. In one embodiment all amino acids in the PYY analogue or derivative thereof for which the optical isomer is not stated is to be understood to mean the L-isomer. In one embodiment at least one of the amino acids in the PYY analogue or derivative thereof are D-amino acids. In one embodiment the constituent amino acids of the PYY analogue or derivative thereof may be selected from at least one of the group of the proteinogenic amino acids encoded by the genetic code and the non-proteinogenic amino acids, such as natural amino acids which are not encoded by the genetic code and synthetic amino acids. In one embodiment the proteinogenic amino acids comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, selenocysteine, and pyrrolysine. Non-proteinogenic amino acids comprise natural amino acids which are not encoded by the genetic code, e.g. D-isomers of the amino acids encoded by the genetic code such as but not limited to D-alanine, D-leucine or D-glutamine; other natural amino acids such as but not limited to γ-carboxyglutamate, ornithine, hydroxyproline or phosphoserine; synthetic chemically manufactured amino acids such as the beta analogues of amino acids such as but not limited to β-alanine; Calpha methylated amino acid such as but not limited to Aib (α-aminoisobutyric acid), Calpha-methyl Phe or C-alpha-methyl Tyr; N-methyl amino acids, such as but not limited to N-methyl Asn, N-methyl H is, N-methyl Arg or N-methyl Tyr; homo amino acids such as but not limited to homohistidine, homoglutamine, homoarginine (2-Amino-6-guanidino-hexanoic acid, HomoArg), homoleucine or homophenylalanine, beta-homo amino acid such as but not limited to β-homo Gln; nor amino acids such as but not limited to norleucine, diamino acids such as but not limited to diaminopropionic acid, 2,4-diaminobutyric acid or ornithine; N-substituted glycines such as but not limited to (2-Carbamoyl-ethylamino)-acetic acid (NGln) or (3-Guanidino-propylamino)-acetic acid (NArg); amino acids with methylated sidechain functional groups such as but not limited to N-epsilon-methyllysine, (N-epsilon, N-epsilon)dimethyllysine, (N-epsilon, N-epsilon, N-epsilon)trimethyllysine, N-omega-methylarginine or (N-omega, N-omega)dimethylarginine; amino acids with shortened sidechains such as but not limited to 2-amino-3-guanidino-propionic acid (Agp), 2-amino-4-guanidino-butyric acid (Agb); other arginine mimicking amino acids such as but not limited to (2-Guanidino-ethylamino)acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or amino-(1-carbamimidoyl-piperidin-4-yl)acetic acid; or other synthetic amino acids such as but not limited to 3-pyridylalanine, (4-Hydroxy-benzylamino)-acetic acid, Abu (α-aminobutyric acid), Tle (tert-butylglycine), 3-aminomethyl benzoic acid, anthranilic acid, pyridylalanine, 2-pyridylalanine, 4-pyridylalanine, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, (1-aminocyclooctyl) carboxylic acid, thiotyrosine, 3-mercaptophenylalanine, 3- or 4-aminophenylalanine, 3- or 4-acetylphenylalanine, 2- or 3-hydroxyphenylalanine (o- or m-tyrosine), hydroxymethylglycine, aminoethylglycine, 1-methyl-1-mercaptoethylglycine, aminoethylthioethylglycine, 2-amino-histidine, β-hydroxy-histidine or mercaptoethylglycine etc. Many of the non-proteinogenic amino acids useful in the present invention are commercially available. Others may be prepared by methods known in the art. In one embodiment a non-proteinogenic amino acid is a moiety which can be incorporated into a peptide via peptide bonds but is not a proteogenic amino acid. In one embodiment the constituent amino acids of the PYY analogue or derivative thereof according to the invention may be selected from at least one of the group of the amino acids encoded by the genetic code, proteinogenic amino acids which are not encoded by the genetic code and synthetic amino acids.

In one embodiment the invention involves the use of N-substituted glycines, which are a specific subclass of peptidomimetics. The N-substituted glycines are closely related to proteinogenic or non-proteinogenic amino acid counterpart, but differ chemically in that their side chains are appended to nitrogen atoms along the backbone of the molecule, rather than to the α-carbons (as they are in amino acids). For convenience, we have given the monomers of N-substituted glycines designations corresponding to the amino acids with similar functionality, with the prefix N. As examples NArg is intented to mean N-substituted glycine with an arginine sidechain and NGln is intented to mean N-substituted glycine with a glutamine sidechain, see formulas for NArg and NGln below.

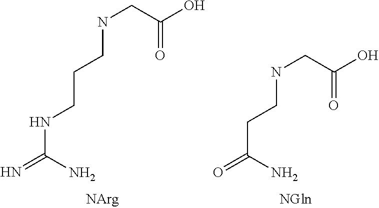 [SMILES: N=C(N)NCCCNCC(=O)O ; NC(=O)CCNCC(=O)O]

In one embodiment the invention involves the use of arginine mimics, which are closely related to proteinogenic or non-proteinogenic amino acid counterpart, but differ chemically in that their side chains are either elongated or truncated. For convenience, we have given the monomers of arginine mimics the following designations: Agp is intented to mean 2-amino-3-guanidino-propionic acid in which the guanidino group containing sidechain has been shortened by two carbon atoms; Agb is intented to mean 2-amino-4-guanidino-butyric acid in which the guanidino group containing sidechain has been shortened by one carbon atom; and homoarginine or HomoArg is intented to mean 2-Amino-6-guanidino-hexanoic acid in which the guanidino group containing sidechain has been elongated by one carbon atom.

In one embodiment the PYY analogue or derivative thereof does not include non-proteinogenic amino acid residues. In one embodiment the PYY analogue or derivative thereof include only L-amino acid residues and/or modified proteinogenic L-amino acid residues.

In one embodiment the PYY analogue or derivative thereof comprises at least one amino acid residue which is substituted with proteinogenic and non-proteinogenic amino acids including but not limited to (both the D- and L-configuration are contemplated, however, for convenience only the L-configuration is shown):

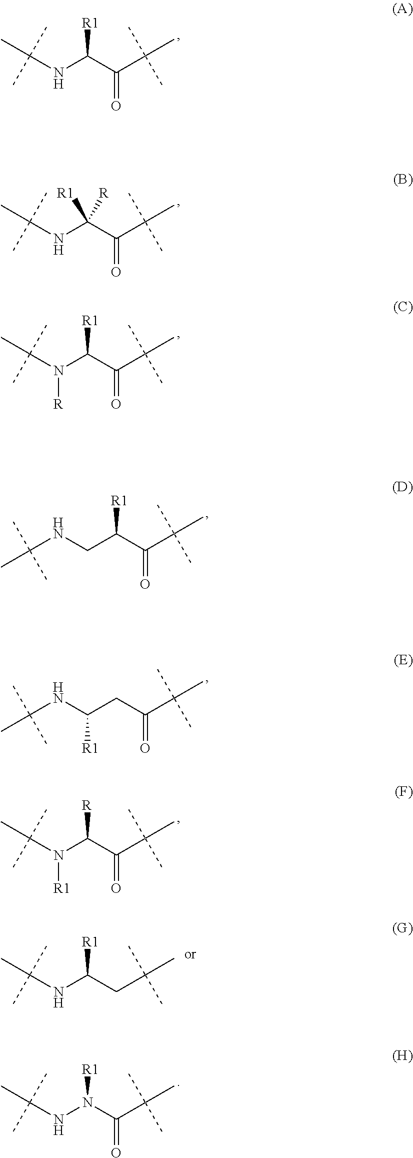

wherein, R1 is intented to mean the functional amino acid sidechain selected from the group consisting of

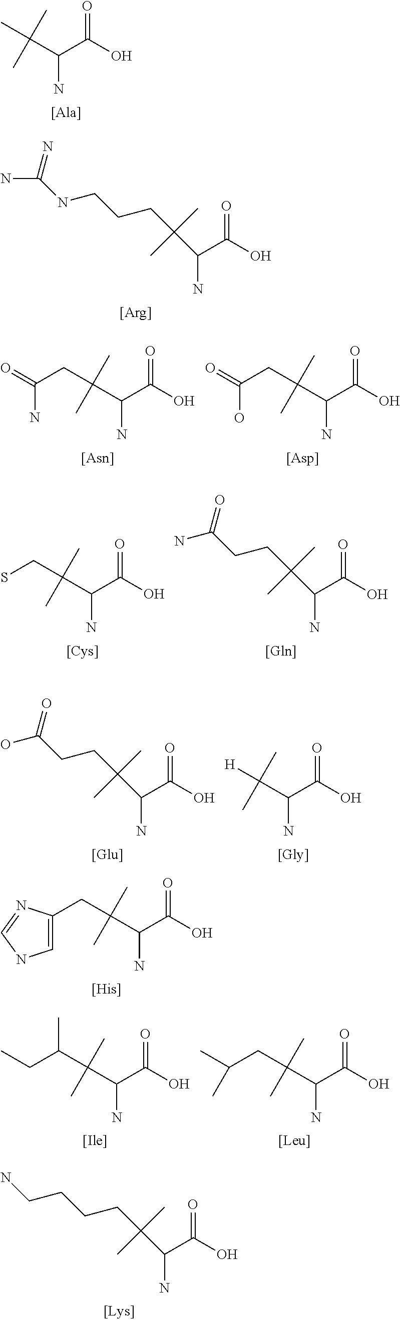

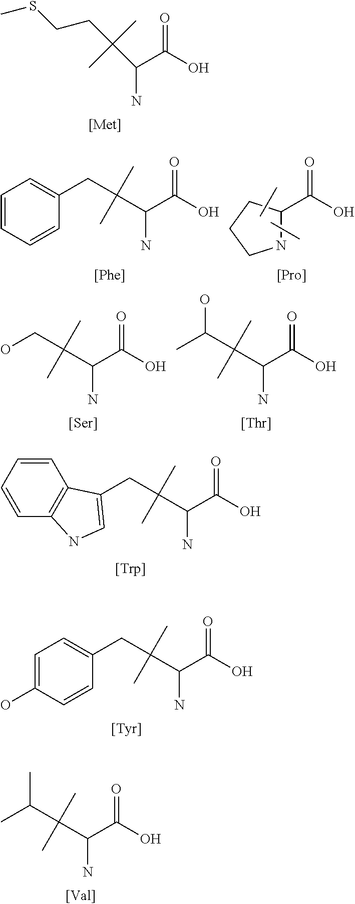

wherein the dashed line represents the disconnection between the functional sidechain and the amino acid backbone; and
R is selected from the group comprising H and C1-C12 alkyl.

In one embodiment the PYY analogue or derivative thereof comprises the amino acid residue represented by formula (C) in at least one position of the peptide backbone of said analogue or derivative. In one embodiment the amino acid residue of formula (C) is found at least one position selected from the group consisting of position 33, 34, 35 and 36. In one embodiment the amino acid residue of formula (C) is found in position 35. In one embodiment when the amino acid residue of formula (C) is found in position 35 then R is the amino acid side chain of Arg and R is selected from the group consisting of H, alkyl (such as C1-C12 alkyl), benzyl or phenyl.

In one embodiment the PYY analogue or derivative thereof comprises at least one peptide bond which is altered to a reduced peptide bond or a peptide bond isoster selected from at least one from the group consisting of a tetrazole, a sulphoneamide and an azide.

In one embodiment a maximum of 8 amino acids have been substituted, deleted, inserted and/or modified in the PYY analogue or derivative thereof as compared to human PYY(1-36). In one embodiment a maximum of 7 amino acids have been substituted, deleted, inserted and/or modified in the PYY analogue or derivative thereof as compared to human PYY(1-36). In one embodiment a maximum of 6 amino acids have been substituted, deleted, inserted and/or modified in the PYY analogue or derivative thereof as compared to human PYY(1-36). In one embodiment a maximum of 5 amino acids have been substituted, deleted, inserted and/or modified in the PYY analogue or derivative thereof as compared to human PYY(1-36). In one embodiment a maximum of 4 amino acids have been substituted, deleted, inserted and/or modified in the PYY analogue or derivative thereof as compared to human PYY(1-36). In one embodiment a maximum of 3 amino acids have been substituted, deleted, inserted and/or modified in the PYY analogue or derivative thereof as compared to human PYY(1-36). In one embodiment a maximum of 2 amino acids have been substituted, deleted, inserted and/or modified in the PYY analogue or derivative thereof as compared to human PYY(1-36). In one embodiment 1 amino acid has been substituted, deleted, inserted and/or modified in the PYY analogue or derivative thereof as compared to human PYY(1-36).

In one embodiment the PYY analogue or derivative thereof exhibits at least 60%, 65%, 70%, 80%, or 90% sequence identity to PYY(1-36) or PYY(3-36) over the entire length of the PYY(1-36) or PYY(3-36), respectively. As an example of a method for determination of sequence identity between two analogues the two peptides [Ala34]PYY(1-36) and PYY(1-36) are aligned. The sequence identity of Ala34 analogue relative to PYY(1-36) is given by the number of aligned identical residues minus the number of different residues divided by the total number of residues in PYY(1-36). Accordingly, in said example the sequence identity is (36-1)/36.

In one embodiment the PYY analogue or derivative thereof comprises at least one alteration, such as at least one of substitution, insertion, deletion and modification. In one embodiment the PYY analogue or derivative thereof includes at least one substitution, insertion, deletion and modification of a “non-essential” amino acid residue. A “nonessential” amino acid residue is intended to mean a residue that can be altered, i.e., deleted or substituted, in the sequence of the peptide without abolishing or substantially reducing the activity of said peptide. In one embodiment “activity” of the PYY analogue or derivative thereof of the invention is Y2 receptor potency as is determined by a Y2 receptor potency assay, such as Assay (I) described herein, Y2 receptor ACTOne assay. The term “substitution” is intended to mean the change of one amino acid in the native sequence with another amino acid. The term “deletion” is intended to mean the removal of one or more amino acids form the native sequence. The term “insertion” is intended to mean the addition of one or more amino acid into the native sequence. The term “modification” is intended to mean alterations covalently attached to the side chain of one or more amino acids or the alpha nitrogen atom of one or more amino acid in the native peptifde sequence.

In one embodiment the C-terminal of the derivative according to the invention may be terminated as either an acid or amide. In one embodiment the C-terminal of the derivative of the invention is an amide.

Substitutions.

In one embodiment the PYY analogue or derivative thereof has at least one substitution in the amino acid sequence compared to human PYY(1-36), alone or in combination with at least one insertion or deletion. In one embodiment the substitution does not abolish or substantially reduce activity of the PYY analogue or derivative thereof. In one embodiment the PYY analogue or derivative thereof has a single substitution or a consecutive or non-consecutive substitution of more than one amino acid residues compared to the amino acid sequence of human PYY(1-36). In one embodiment the PYY analogue or derivative thereof includes one, two or three amino acid substitutions compared to the amino acid sequence of human PYY(1-36).

In one embodiment the amino acid residues of at the helical C-terminus region of PYY (e.g., residues 20, 24, 25, 27 and 29), the tail end residues (32-36), and/or the N-terminus prolines at position 5 and 8 are not substituted. In one embodiment amino acid residues are not substituted at positions 32 through 36 of PYY. In one embodiment amino acid residues of PYY are not substituted at at least one amino acid sequence position selected from: 5, 7, 8, 20, 24, 25, 27, 29, 32, 33, 34, 35, 36 and any combination thereof.

In one embodiment amino acids are substituted by conservative substitution. The term “conservative substitution” as used herein denotes that at least one amino acid is replaced by at least one biologically similar residue. Examples include substitution of amino acid residues with similar characteristics, e.g., small amino acids, acidic amino acids, polar amino acids, basic amino acids, hydrophobic amino acids and aromatic amino acids. For example, in one embodiment Met residues are substituted with norleucine (Nle) or with leucine, isoleucine or valine, which—as opposed to Met—are not readily oxidised. Another example of a conservative substitution with a residue normally not found in endogenous, mammalian peptides and proteins would be the conservative substitution of Arg or Lys with for example, ornithine, canavanine, aminoethylcysteine or another basic amino acid. For further information concerning phenotypically silent substitutions in peptides and proteins, see, e.g., Bowie et. al. Science 247, 1306-1310, 1990. Conservatively substituted analogues of the invention may have, e.g., up to 10 conservative substitutions, such as up to 5 or such as 3 or fewer conservative substitutions.

In one embodiment the PYY analogue or derivative thereof include substitutions of at least one non-proteinogenic and/or non-amino acid, e.g., amino acid mimetics, into the sequence of PYY. In one embodiment the non-amino acids inserted into the sequence of PYY are beta-turn mimetics or molecules, such as —NH—X—CO—, wherein X═(CH2)ₙ (where n can be 2-20) or —NH—CH2CH2(—O—CH2CH2-O—)ₘ—CH2-CO—(where m=1-5). In one embodiment molecules inserted into the sequence of PYY may be selected from aminocaproyl (“Aca”), beta-alanyl, and 8-amino-3,6-dioxaoctanoyl. beta-turn mimetics are available commercially (BioQuadrant Inc, Quebec, Canada).

Deletions.

In one embodiment the PYY analogue or derivative thereof has at least one amino acid residue deleted from the amino acid sequence of human PYY(1-36), alone or in combination with at least one insertion or substitution. In one embodiment the PYY analogue or derivative thereof has at least one amino acid residue deleted at amino acid positions 4 through 35 of PYY. Such deletions may include at least one consecutive or non-consecutive deletion at amino acid positions 4 through 35 of PYY. In one embodiment the amino acid residues at positions 24 through 36 of PYY are not deleted.

In one embodiment the PYY analogue or derivative thereof includes N-terminal or C-terminal truncations, or internal deletions at amino acid positions 4 to 35 as long as at least one biological activity of human PYY(1-36) is retained. In one embodiment positions 1 and 2 of PYY are deleted. In one embodiment the amino acid residues at positions 5 through 8 and 24 through 36, more specifically positions 5 through 8 and positions 32 through 35 of PYY are not deleted.

Insertions.

In one embodiment the PYY analogue or derivative thereof has at least one amino acid residue inserted into the amino acid sequence of human PYY(1-36), alone or in combination with at least one deletion and/or substitution. In one embodiment the PYY analogue or derivative thereof has a single insertion, or consecutive or non-consecutive insertions of more than one amino acid residues into the amino acid sequence of human PYY(1-36). In one embodiment at least one amino acid is inserted at the N-terminal or C-terminal end of the PYY analogue or derivative thereof. In one embodiment amino acid residues are not inserted at positions 24 through 36 of PYY.

In one embodiment the PYY analogue or derivative thereof comprises chemical alterations to at least one amino acid residue. Such chemical alterations include amidation, glycosylation, acylation, sulfation, phosphorylation, acetylation and/or cyclization. The chemical alterations may occur singularly at the N- or C-terminus or at the side chains of amino acid residues within the sequence of the PYY analogue or derivative thereof. In one embodiment the C-terminus of these peptides may have a free —OH or —NH₂ group. In one embodiment the N-terminal end of the peptides may be capped with an isobutyloxycarbonyl group, an isopropyloxycarbonyl group, an n-butyloxycarbonyl group, an ethoxycarbonyl group, an isocaproyl group (isocap), an octanyl group, an octyl glycine group (G(Oct)), an 8-aminooctanic acid group or a Fmoc group. In one embodiment there are multiple sites of chemical alteration along the peptide of the PYY analogue or derivative thereof.

In one embodiment the PYY analogue or derivative thereof include insertions of at least one non-proteinogenic amino acid and/or non-amino acid into the sequence of human PYY(1-36). In one embodiment the non-proteinogenic amino acids inserted into the sequence of human PYY(1-36) may be beta-turn mimetics or molecules inserted into the sequence of PYY. Examples of molecules inserted into the sequence of PYY include aminocaproyl (“Aca”), beta-alanyl and 8-amino-3,6-dioxaoctanoyl.

In one embodiment the PYY analogue or derivative thereof comprises combinations of two or more changes selected from the group consisting of deletion, insertion, and substitution. In one embodiment the PYY analogue or derivative thereof comprises one, two or three amino acid substitutions. In one embodiment the PYY analogue or derivative thereof comprises one, two or three amino acid modifications.

In one embodiment the PYY analogue or derivative thereof has an improved enzymatic stability compared to human PYY(3-36) or human PYY(3-36) with a serum albumin binding side chain identical to that of said analogue or derivative. In one embodiment the enzymatic stability is determined by Assay (IV), in vitro half life in plasma, described herein.

In one embodiment the PYY analogue or derivative thereof comprises the amino acid sequence of formula (I):

Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅-Xaa₂₆-Xaa₂₇-Xaa₂₈-Xaa₂₉-Xaa₃₀-Xaa₃₁-Xaa₃₂-Xaa₃₃-Xaa₃₄-Xaa₃₅-Xaa₃₆ Formula (I)

wherein
Xaa₁ is Tyr, Phe, Ala, 2,3-diaminopropionic acid, 2,4-diaminobutyric acid, ornithine, Lys or absent;
Xaa₂ is Pro, Ala, Leu, Phe, hydroxyproline, 2,3-diaminopropionic acid, 2,4-diaminobutyric acid, ornithine, Lys or absent;
Xaa₃ is Ile, Val, Leu (1-aminocyclopentyl) carboxylic acid, (1-aminocyclohexyl) carboxylic acid, 1-aminobutyric acid, 2,3-diaminopropionic acid, 2,4-diaminobutyric acid, ornithine, Lys;
Xaa₄ is Lys, Glu, 2,3-diaminopropionic acid, 2,4-diaminobutyric acid, ornithine, or Lys;
Xaa₅ is Pro, 2,3-diaminopropionic acid, 2,4-diaminobutyric acid, ornithine, or Lys;
Xaa₆ is Glu, 2,3-diaminopropionic acid, 2,4-diaminobutyric acid, ornithine, or Lys;
Xaa₇ is Ala, 2,3-diaminopropionic acid, 2,4-diaminobutyric acid, ornithine, or Lys;
Xaa₈ is Pro, Ala, 2,3-diaminopropionic acid, 2,4-diaminobutyric acid, ornithine, or Lys;
Xaa₉ is Gly, 2,3-diaminopropionic acid, 2,4-diaminobutyric acid, ornithine, or Lys;
Xaa₁₀ is Glu, 2,3-diaminopropionic acid, 2,4-diaminobutyric acid, ornithine or Lys;
Xaa₁₁ is Asp, Ala, 2,3-diaminopropionic acid, 2,4-diaminobutyric acid, ornithine or Lys;
Xaa₁₂ is Ala, 2,3-diaminopropionic acid, 2,4-diaminobutyric acid, ornithine or Lys;
Xaa₁₃ is Ser, 2,3-diaminopropionic acid, 2,4-diaminobutyric acid, ornithine or Lys;
Xaa₁₄ is Pro or hydroxyproline;
Xaa₁₅ is Glu, 2,3-diaminopropionic acid, 2,4-diaminobutyric acid, ornithine or Lys;
Xaa₁₆ is Glu, 2,3-diaminopropionic acid, 2,4-diaminobutyric acid, ornithine or Lys;
Xaa₁₇ is Leu, Val, Ile, homoleucine, norleucine, (1-aminocyclopentyl) carboxylic acid, (1-aminocyclohexyl) carboxylic acid, or 1-aminobutyric acid;
Xaa₁₈ is Asn, 2,3-diaminopropionic acid, 2,4-diaminobutyric acid, ornithine or Lys;
Xaa₁₉ is Arg, Ala, 2,3-diaminopropionic acid, 2,4-diaminobutyric acid, ornithine or Lys;
Xaa₂₀ is Tyr, Ala, Phe, 3-pyridylalaine, 2,3-diaminopropionic acid, 2,4-diaminobutyric acid, ornithine or Lys;
Xaa₂₁ is Tyr, Ala, Phe, 3-pyridylalaine, 2,3-diaminopropionic acid, 2,4-diaminobutyric acid, ornithine or Lys;
Xaa₂₂ is Ala, 2,3-diaminopropionic acid, 2,4-diaminobutyric acid, ornithine or Lys;
Xaa₂₃ is Ser, 2,3-diaminopropionic acid, 2,4-diaminobutyric acid, ornithine or Lys;
Xaa₂₄ is Leu, Ile, Val, homoleucine, norleucine, (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₂₅ is Arg, Ala, His, aminoisobutyric acid, 2,3-diaminopropionic acid, 2,4-diaminobutyric acid, ornithine or Lys;
Xaa₂₆ is His, Arg, 2,3-diaminopropionic acid, 2,4-diaminobutyric acid, ornithine or Lys;
Xaa₂₇ is Tyr, Ala, Phe, homoPhe or 3-pyridylalanine, 2,3-diaminopropionic acid, 2,4-diaminobutyric acid, ornithine or Lys;
Xaa₂₈ is Ile, Val, Leu, homoleucine, norleucine, (1-aminocyclopentyl) carboxylic acid, (1-aminocyclohexyl) carboxylic acid, aminoisobutyric acid, 1-aminobutyric acid, 2,3-diaminopropionic acid, 2,4-diaminobutyric acid, ornithine or Lys;
Xaa₂₉ is Asn, Gln, 2,3-diaminopropionic acid, 2,4-diaminobutyric acid, ornithine or Lys;
Xaa₃₀ is Met, Leu, Val, Ile, homoleucine, aminoisobutyric acid, norleucine, (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₃₁ is Leu, Val, Ile, aminoisobutyric acid, homoleucine, norleucine, (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₃₂ is Ser, Thr, 2,3-diaminopropionic acid, 2,4-diaminobutyric acid, ornithine or Lys;
Xaa₃₃ is Arg, N-methyl Arg, methyllysine, dimethyllysine, trimethyllysine, 2-amino-3-guanidino-propionic acid (Apg), 2-amino-4-guanidino-butyric acid (Abg), monomethyllarginine, dimethylarginine, (2-Guanidino-ethylamino)-acetic acid, (3-Guanidino-propylamino)-acetic acid (NArg), (4-Guanidino-butylamino)-acetic acid, 2-Amino-3-(1-carbamimidoyl-pyrrolidin-2-yl)-propionic acid, 2-Amino-4-(2-amino-pyrimidin-4-yl)butyric acid, 2-Amino-3-(4-guanidino-phenyl)-propionic acid, or Amino-(1-carbamimidoyl-piperidin-4-yl)-acetic acid;
Xaa₃₄ is Gln, Asn, His, Pro, N-methyl Gln, β-homo Gln, (2-Carbamoyl-ethylamino)-acetic acid (NGln), N-methyl Asn, or N-methyl His;
Xaa₃₅ is Arg, N-methyl Arg, methyllysine, dimethyllysine, trimethyllysine, 2-amino-3-guanidino-propionic acid (Agp), 2-amino-4-guanidino-butyric acid (Agb), homoarginine (2-Amino-6-guanidino-hexanoic acid, HomoArg), monomethylarginine, dimethylarginine, (2-Guanidino-ethylamino)-acetic acid, (3-Guanidino-propylamino)-acetic acid (NArg), (4-Guanidino-butylamino)-acetic acid, 2-Amino-3-(1-carbamimidoyl-pyrrolidin-2-yl)propionic acid, 2-Amino-4-(2-amino-pyrimidin-4-yl)-butyric acid, 2-Amino-3-(4-guanidino-phenyl)-propionic acid, or Amino-(1-carbamimidoyl-piperidin-4-yl)-acetic acid;
Xaa₃₆ is Tyr, Phe, N-methyl Tyr, C-α-methyl Phe, 3-pyridylalanine, (4-Hydroxybenzylamino)-acetic acid (NTyr).

In one embodiment Xaa₁ is Tyr. In one embodiment Xaa₁ is Lys.

In one embodiment Xaa₂ is Pro.

In one embodiment Xaa₃ is Ile. In one embodiment Xaa₃ is Lys.

In one embodiment Xaa₄ is Lys. In one embodiment Xaa₄ is Lys. In one embodiment Xaa₄ is Arg. In one embodiment Xaa₄ is Asp. In one embodiment Xaa₄ is Glu.

In one embodiment Xaa₅ is Pro. In one embodiment Xaa₅ is Lys.

In one embodiment Xaa₆ is Glu. In one embodiment Xaa₆ is Lys.

In one embodiment Xaa₇ is Ala. In one embodiment Xaa₇ is Lys.

In one embodiment Xaa₈ is Pro. In one embodiment Xaa₈ is Lys.

In one embodiment Xaa₉ is Gly. In one embodiment Xaa₉ is Lys.

In one embodiment Xaa₁₀ is Glu. In one embodiment Xaa₁₀ is Lys.

In one embodiment Xaa₁₁ is Asp. In one embodiment Xaa₁₁ is Lys. In one embodiment Xaa 11 is Glu.

In one embodiment Xaa₁₂ is Ala. In one embodiment Xaa₁₂ is Lys.

In one embodiment Xaa₁₃ is Ser. In one embodiment Xaa₁₃ is Lys.

In one embodiment Xaa₁₄ is Pro. In one embodiment Xaa₁₅ is Lys.

In one embodiment Xaa₁₅ is Glu. In one embodiment Xaa₁₆ is Lys.

In one embodiment Xaa₁₆ is Glu. In one embodiment Xaa₁₇ is Lys.

In one embodiment Xaa₁₇ is Leu. In one embodiment Xaa₁₈ is Lys.

In one embodiment Xaa₁₈ is Ala. In one embodiment Xaa₁₈ is Lys. In one embodiment Xaa₁₈ is Ala. In one embodiment Xaa₁₈ is Glu. In one embodiment Xaa₁₈ is Gln.

In one embodiment Xaa₁₉ is Arg. In one embodiment Xaa₁₉ is Lys.

In one embodiment Xaa₂₀ is Tyr. In one embodiment Xaa₂₀ is Lys. In one embodiment Xaa 20 is Ala.

In one embodiment Xaa₂₁ is Tyr. In one embodiment Xaa₂₁ is Lys.

In one embodiment Xaa₂₂ is Ala. In one embodiment Xaa₂₂ is Lys. In one embodiment Xaa₂₂ is Asp. In one embodiment Xaa₂₂ is Glu.

In one embodiment Xaa₂₃ is Ser. In one embodiment Xaa₂₃ is Lys.

In one embodiment Xaa₂₄ is Leu. In one embodiment Xaa₂₄ is Lys.

In one embodiment Xaa₂₅ is Arg. In one embodiment Xaa₂₅ is Lys. In one embodiment Xaa₂₅ is Aib. In one embodiment Xaa 25 is His. In one embodiment Xaa 25 is Aib. In one embodiment Xaa 25 is Tyr.

In one embodiment Xaa₂₆ is His. In one embodiment Xaa₂₆ is Lys.

In one embodiment Xaa₂₇ is Tyr. In one embodiment Xaa₂₇ is Lys. In one embodiment Xaa 27 is Ala.

In one embodiment Xaa₂₈ is Leu. In one embodiment Xaa₂₈ is Lys.

In one embodiment Xaa₂₉ is Asn. In one embodiment Xaa₂₉ is Lys. In one embodiment Xaa₂₉ is Gln.

In one embodiment Xaa₃₀ is Leu. In one embodiment Xaa₃₀ is Lys.

In one embodiment Xaa₃₁ is Val. In one embodiment Xaa₃₁ is Lys.

In one embodiment Xaa₃₂ is Thr. In one embodiment Xaa₃₂ is Lys.

In one embodiment Xaa₃₃ is Arg. In one embodiment Xaa₃₃ is Lys. In one embodiment Xaa₃₃ is N-methyl Arg. In one embodiment Xaa₃₃ is methyllysine. In one embodiment Xaa₃₃ is dimethyllysine. In one embodiment Xaa₃₃ is trimethyllysine. In one embodiment Xaa₃₃ is 2-amino-3-guanidino-propionic acid (Apg). In one embodiment Xaa₃₃ is 2-amino-4-guanidino-butyric acid (Abg). In one embodiment Xaa₃₃ is monomethylarginine. In one embodiment Xaa₃₃ is dimethylarginine. In one embodiment Xaa₃₃ is (2-guanidino-ethylamino)-acetic acid. In one embodiment Xaa₃₃ is (3-guanidino-propylamino)-acetic acid (NArg). In one embodiment Xaa₃₃ is (4-guanidino-butylamino)acetic acid. In one embodiment Xaa₃₃ is 2-amino-3-(1-carbamimidoyl-pyrrolidin-2-yl)propionic acid. In one embodiment Xaa₃₃ is 2-amino-4-(2-amino-pyrimidin-4-yl)-butyric acid. In one embodiment Xaa₃₃ is 2-amino-3-(4-guanidino-phenyl)-propionic acid. In one embodiment Xaa₃₃ is amino-(1-carbamimidoyl-piperidin-4-yl)-acetic acid.

In one embodiment Xaa₃₄ is Gln. In one embodiment Xaa₃₄ is Asn. In one embodiment Xaa₃₄ is His. In one embodiment Xaa₃₄ is Pro. In one embodiment Xaa₃₄ is Lys. In one embodiment Xaa₃₄ is N-methyl Gln. In one embodiment Xaa₃₄ is β-homo Gln. In one embodiment Xaa₃₄ is (2-carbamoyl-ethylamino)-acetic acid (NGln). In one embodiment Xaa₃₄ is N-methyl Asn. In one embodiment Xaa₃₄ is N-methyl His.

In one embodiment Xaa₃₅ is Arg. In one embodiment Xaa₃₅ is Lys. In one embodiment Xaa₃₅ is N-methyl Arg. In one embodiment Xaa₃₅ is methyllysine. In one embodiment Xaa₃₅ is dimethyllysine. In one embodiment Xaa₃₅ is trimethyllysine. In one embodiment Xaa₃₅ is 2-amino-3-guanidino-propionic acid (Agp). In one embodiment Xaa₃₅ is 2-amino-4-guanidino-butyric acid (Agb). In one embodiment Xaa₃₅ is homoarginine (2-amino-6-guanidino-hexanoic acid, HomoArg). In one embodiment Xaa₃₅ is monomethylarginine. In one embodiment Xaa₃₅ is dimethylarginine. In one embodiment Xaa₃₅ is (2-guanidino-ethylamino)-acetic acid. In one embodiment Xaa₃₅ is (3-guanidino-propylamino)-acetic acid (NArg). In one embodiment Xaa₃₅ is (4-guanidino-butylamino)acetic acid. In one embodiment Xaa₃₅ is 2-amino-3-(1-carbamimidoyl-pyrrolidin-2-yl)propionic acid. In one embodiment Xaa₃₅ is 2-amino-4-(2-amino-pyrimidin-4-yl)-butyric acid. In one embodiment Xaa₃₅ is 2-amino-3-(4-guanidino-phenyl)-propionic acid. In one embodiment Xaa₃₅ is amino-(1-carbamimidoyl-piperidin-4-yl)-acetic acid.

In one embodiment Xaa₃₆ is Tyr. In one embodiment Xaa₃₆ is Phe. In one embodiment Xaa₃₆ is N-methyl Tyr. In one embodiment Xaa₃₆ is C-α-methyl Phe. In one embodiment Xaa₃₆ is 3-pyridylalanine. In one embodiment Xaa₃₆ is (4-Hydroxybenzylamino)-acetic acid (NTyr).

In one embodiment Xaa₁ and Xaa₂ are absent. In one embodiment Xaa₁, Xaa₂, Xaa₃ and Xaa₄ are absent.

In one embodiment the PYY analogue or a derivative thereof is not [N-methyl Arg33] PYY(3-36), [N-methyl Gln34] PYY(3-36), [N-methyl Arg35] PYY(3-36), [N-methyl Tyr36] PYY(1-36), or [N-methyl Tyr36] PYY(3-36).

In one embodiment the PYY analogue or a derivative thereof comprises an amino acid substitution in at least one position selected from the group consisting of position 33, 34, 35 and 36.

### Derivatives of PYY Analogues

In one embodiment the PYY analogue or derivative obtains protracted pharmacokinetic properties by attachment of said analogue or derivative to serum albumin in vivo. Said attachment can be either covalent or non-covalent. In one embodiment fatty acids are attached to the PYY analogue or derivative causing it to bind non-covalently to albumin. In one embodiment this invention describes the design of peptides comprising attachment of serum albumin binding side chains that strongly bind to albumin and prolong the duration of action of the peptide drug with the result that the peptide drug can be dosed less frequently, such as once-daily or more rarely, than, e.g., human PYY(3-36). In one embodiment the PYY analogue or derivative thereof comprises attachment of at least one side chain that strongly bind to albumin, i.e. a serum albumin binding side chain. In one embodiment the serum albumin binding side chain comprises a distal carboxylic acid group or a distal tetrazole group. In one embodiment the serum albumin binding side chain comprises a fatty diacid. The terms “serum albumin binding side chain”, “serum albumin binding group” and “albumin binding group” are used interchangeably herein. In one embodiment term “albumin” is intended to mean “serum albumin”.

In one embodiment the serum albumin binding side chain comprises an alkyl chain with at least 14 carbon atoms. In one embodiment the serum albumin binding side chain comprises at least 14 carbon atoms, such as 16, 18 or 20 carbon atoms. In one embodiment the serum albumin binding side chain comprises an amide. In one embodiment the serum albumin binding side chain comprises a distal carboxylic acid group or a distal tetrazole group. In one embodiment the alkyl chain comprises a distal carboxylic acid group or a distal tetrazole group. In one embodiment the serum albumin binding side chain comprises an alkyl chain with at least 14 carbon atoms, wherein said alkyl chain comprises a distal carboxylic acid or a distal tetrazole group. In one embodiment the serum albumin binding side chain comprises a C18 dicarboxylic acid or a C16 dicarboxylic acid. In one embodiment the serum albumin binding side chain comprises a C16 carboxylic acid.

The term “derivative” and the related terms “derivatised” and “derivatisation” as used herein in relation to a peptide means a chemically altered peptide, wherein at least one substituent is not present in the non-altered peptide, i.e. a peptide which has been covalently altered. Typical alterations are amides, carbohydrates, alkyl groups, acyl groups, esters and the like. In one embodiment at least one serum albumin binding side chain is present at the N-terminus, C-terminus or at the side chains of amino acid residues in the sequence of the PYY analogue or derivative thereof. In one embodiment additional sites for derivatisation are provided by substitution of at least one amino acid with lysine, aspartic acid, glutamic acid or cysteine. In one embodiment the PYY analogue or derivative thereof are conjugated to one, two or three serum albumin binding side chain molecules.

In one embodiment any amino acid position in the PYY analogue or derivative thereof may be derivatised. In one embodiment the amino acid residue which is derivatised comprises an amino group. In one embodiment the amino acid residue which is derivatised comprises a primary amino group in a side chain. In one embodiment the amino acid residue which is derivatised is lysine. In one embodiment the amino acid residue which is derivatised is cysteine. In one embodiment the PYY analogue or derivative thereof is only derivatised in one position, e.g. only one amino acid residue is derivatised.

In one embodiment the N-terminal position of the PYY analogue or derivative thereof is derivatised. In one embodiment the N-terminal position of the PYY analogue or derivative thereof is acylated. In one embodiment the N-terminal position of the PYY analogue or derivative thereof is derivatised with an albumin binding group comprising CH₃(CH₂)ᵣCO—, wherein r is 16 or 18. In one embodiment the N-terminal position of human PYY(3-36) or an analogue or derivative thereof is derivatised with an albumin binding group comprising CH₃(CH₂)ᵣCO—, wherein r is 16 or 18.

Examples of amino acid residues comprising an amino group is lysine, ornithine, Epsilon-N-alkylated lysine such as Epsilon-N methyllysine, O-aminoethylserine, O-aminopropylserine or longer O-alkylated serines containing a primary or secondary amino group in the side chain. In one embodiment the derivatised amino acid residue comprises a primary amino group in a side chain. Examples of amino acid residues comprising a primary amino group is lysine ornithine, O-aminoethylserine, O-aminopropylserine or longer 0 alkylated serines containing a primary amino group in the side chain.

In one embodiment the term “serum albumin binding side chain” is intended to mean a moiety which has serum albumin binding properties. In one embodiment the term “serum albumin binding” refers to a inherent ability of a compound to bind to circulating serum albumin and said binding is optinally non-covalent. In one embodiment the “serum albumin binding” Various compounds exhibit different ability to bind to albumin. A high ability of a compound to bind to serum albumin results in a high fraction of bound compound while the corresponding unbound fraction in serum will be low.

An example of a method for determination of albumin binding is as follows: Serum albumin binding can be measured by using columns with immobilised serum albumin from human or other species. The affinity of a given peptide can be measured by an altered elution time from the column and the relative affinities between different albumin binding peptides can be established by comparing the elution time profiles. In one method serum albumin peptides can be biotinylated and the binding of the peptide can be determined by enzyme linked immuno assay (ELISA) technique using microtiter plate with immobilised albumin. The visualisation of the binding is done by using avidin or streptavidin conjugated to either horseradish peroxidise or alkaline phosphatase. The relative affinities of different albumin binding peptides can be measured. Other affinity experiments that may be used in the measurement of albumin binding include Biacore analysis and microcalorimetry.

In one embodiment the serum albumin binding side chain is lipophilic. In one embodiment the serum albumin binding side chain is attached to a lysine residue optionally via a spacer by conjugation chemistry such as by alkylation, acylation, ester formation, or amide formation or to a cysteine residue by maleimide coupling. The term “spacer” as used herein means a molecular unit separates a peptide and a serum albumin binding side chain. In one embodiment the term “spacer” as used herein means a spacer that separates a peptide and an albumin binding side chain with a chemical moiety which comprises at least 5 non-hydrogen atoms where 30-50% of these are either N or 0.

In one embodiment the albumin binding side chain is negatively charged at physiological pH. In one embodiment the albumin binding side chain comprises a group which can be negatively charged. In one embodiment the albumin binding side chain comprises a carboxylic acid group.

In one embodiment the albumin binding side chain is selected from the group consisting of a straight chain alkyl group, a branched alkyl group, a group which has an ω-carboxylic acid group, and a partially or completely hydrogenated cyclopentanophenanthrene skeleton.

In one embodiment the albumin binding side chain is a cibacronyl residue.

In one embodiment the albumin binding side chain has from 6 to 40 carbon atoms, from 8 to 26 carbon atoms or from 8 to 20 carbon atoms.

In one embodiment the albumin binding side chain is an acyl group selected from the group comprising CH₃(CH₂)ᵣCO—, wherein r is an integer from 4 to 38, specifically an integer from 4 to 24, more preferred selected from the group comprising CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In one embodiment the albumin binding side chain is an acyl group of a straight-chain or branched alkane α,ω-dicarboxylic acid.

In one embodiment the albumin binding side chain is A-B-C-D- or A-C-D- or A-B-C- or A-C-,

wherein A- is

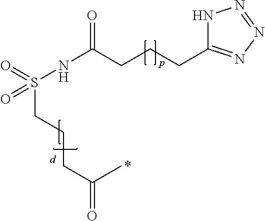

wherein p is selected from the group consisting of 10, 11, 12, 13 and 14, and d is selected from the group consisting of 0, 1, 2, 3, 4 and 5, and

-B- is selected from the group consisting of

and

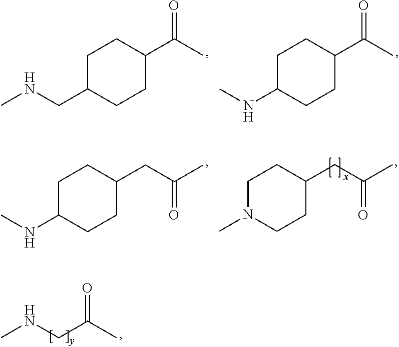

wherein x is selected from the group consisting of 0, 1, 2, 3 and 4, and y is selected from the group consisting of 1, 2, 3, 4, 5, 6, 7, 8, 9, 10, 11 and 12,

or A- is

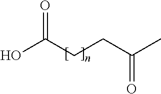

wherein n is selected from the group consisting of 12, 13, 14, 15, 16 17, 18 and 19, and B is selected from the group consisting of

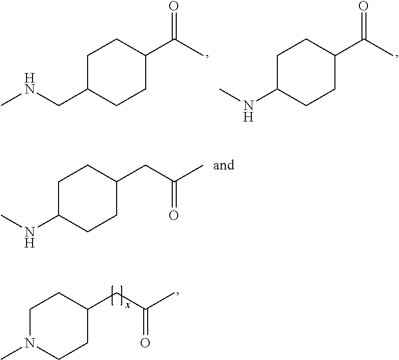

wherein x is selected from the group consisting of 0, 1, 2, 3 and 4, and

-C- is selected from the group consisting of

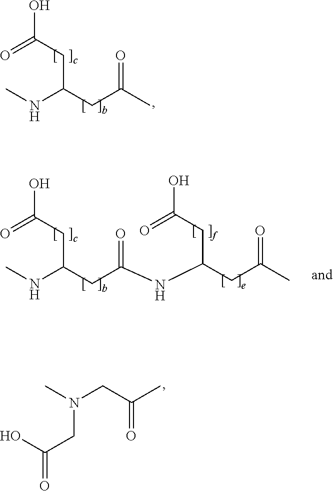

wherein b and e are each independently selected from the group consisting of 0, 1 and 2, and c and f are each independently selected from the group consisting of 0, 1 and 2 with the proviso that b is 1 or 2 when c is 0, or b is 0 when c is 1 or 2, and e is 1 or 2 when f is 0, or e is 0 when f is 1 or 2, and

-D- is attached to said amino acid residue and is a spacer.

In one embodiment the serum albumin binding side chain is attached to the N-terminal amino group, the amino group of the amidated C-terminal or the side chain of an amino acid, such as the side chain of 2,3-diaminopropionic acid, 2,4-diaminobutyric acid, ornithine or Lys.

In one embodiment the serum albumin binding side chain comprises at least one fatty diacid, such as hexadecanedioic acid, octadecanedioic acid or dodecanedioic acid, which due to the negative charge in the distal end of said fatty acid increases affinity of the PYY analogue or derivative thereof to serum albumin. In one embodiment the fatty diacid or tetrazole may be attached to a spacer, such as, but not limited to, a negatively charged amino acid, e.g., L-gamma-glutamate. In one embodiment the fatty diacid or tetrazole may be attached to a hydrophobic spacer such as, but not limited to, tranexamic acid and isonipecotinic acid. In one embodiment the combined fatty diacid or tetrazole and spacer may be separated with a spacer such as, but not limited to, 8-amino-3,6-dioxaoctanoic acid (Oeg) or several Oeg spacers. In one embodiment the serum albumin binding side chain comprises one Oeg molecule. In one embodiment the serum albumin binding side chain comprises two Oeg molecules.

In one embodiment the serum albumin binding side chain is 2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl. In one embodiment the serum albumin binding side chain is 2-(2-{2-[2-(2-{2-[(S)-4-Carboxy-4-(19-carboxynonadecanoylamino)butyrylamino]ethoxy}ethoxy)acetylamino]ethoxy}ethoxy)acetyl. In one embodiment the serum albumin binding side chain is 2-(2-{2-[2-(2-{2-[(S)-4-Carboxy-4-(17-carboxyheptadecanoylamino)butyrylamino]ethoxy}ethoxy)acetyl-amino]ethoxy}ethoxy)acetyl. In one embodiment the serum albumin binding side chain is 2-(2-{2-[2-(2-{2-[(S)-4-Carboxy-4-(15-carboxypentadecanoylamino)butyrylamino]ethoxy}ethoxy)acetylamino]ethoxy}ethoxy)acetyl. In one embodiment the serum albumin binding side chain is 2-(2-{2-[2-(2-{2-[(S)-4-Carboxy-4-(hexadecanoylamino)butyrylamino]ethoxy}ethoxy)acetyl-amino]ethoxy}ethoxy)acetyl. In one embodiment the serum albumin binding side chain is [4-(16-(1H-Tetrazol-5-yl)hexadecanoylsulfamoyl)butyryl]ethoxy}ethoxy)acetylamino]ethoxy}ethoxy)acetyl].

In one embodiment -D- is a spacer providing distance of the albumin handles to the peptide and may be selected from the group consisting of at least one PEG molecule, such as at least two consecutive PEG molecules, at least one glycine, such as at least two consecutive glycines, or other small polar residues. In one embodiment said spacer may comprise at least one 8-amino-3,6-dioxaoctanoic acid (Oeg) molecule, such as at least two consecutive Oeg molecules, or other spacers of the PEG type. In one embodiment said spacer may be a peptide and may comprise or consist of at least two consecutive Gly molecules forming a glycine polymer. In one embodiment the spacer is composed of several polar or hydrophilic amino acids, e.g., but not limited to, (Ser-Gly), where n is an integer; n=1-20 or 1-10 or 1-5. In one embodiment the spacer may be composed of non-alfa-amino acids such as beta-alanine or 8-amino-caprylic acid or combinations thereof.

In one embodiment -D- is

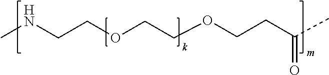

wherein k is selected from the group consisting of 0, 1, 2, 3, 4, 5, 11 and 27, and m is selected from the group consisting of 0, 1, 2, 3, 4, 5 and 6.

In one embodiment A-B-C-D- is selected and combined from any one of:

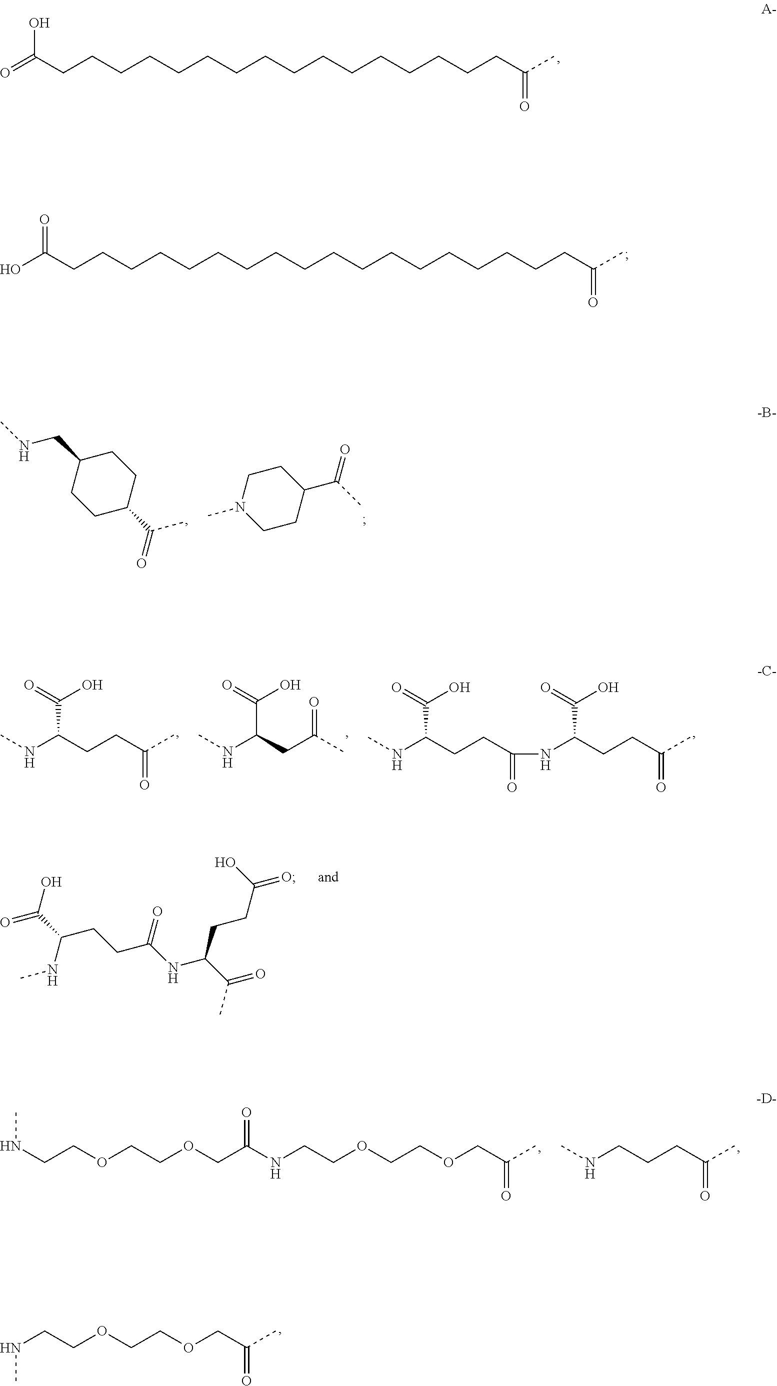

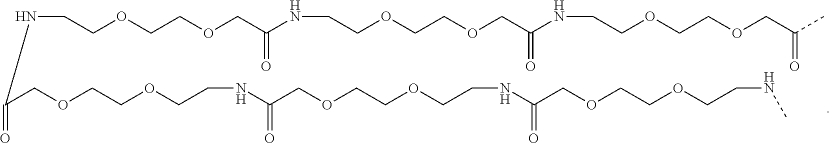

In one embodiment A-B-C-D- is selected and combined from any one of

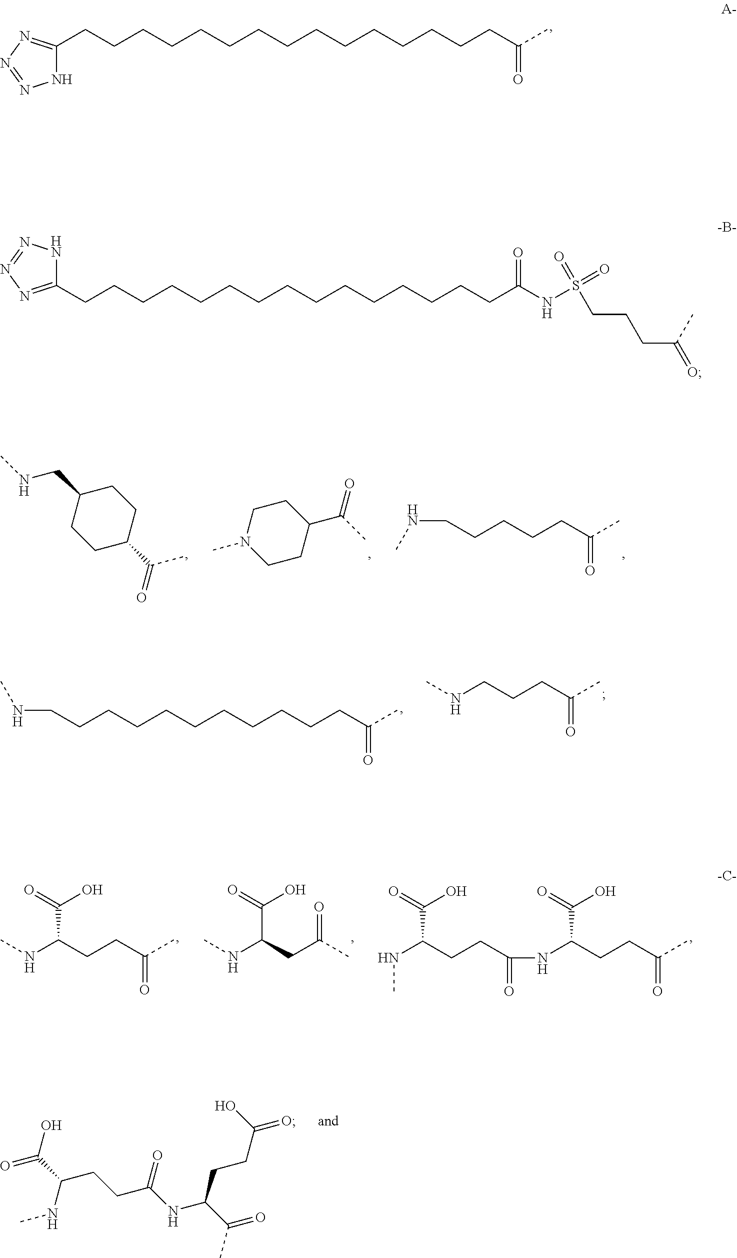

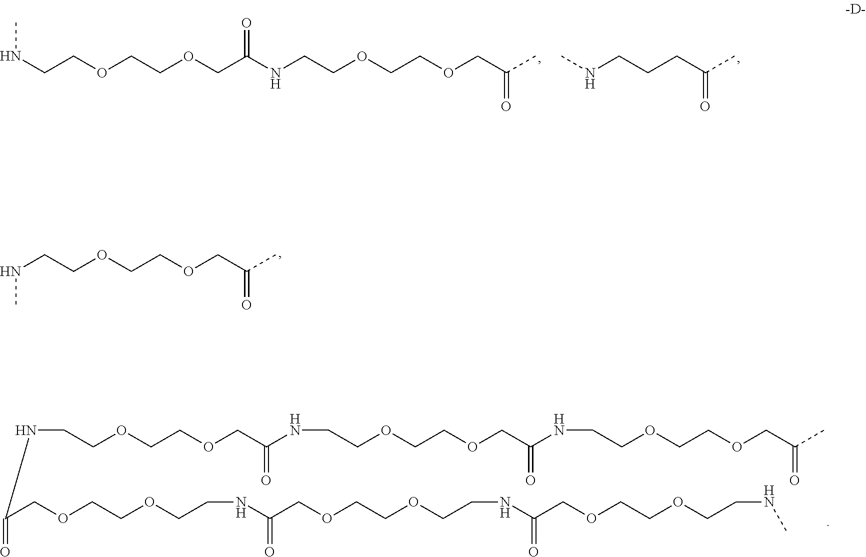

In one embodiment A-B-C-D- is selected from the group consisting of

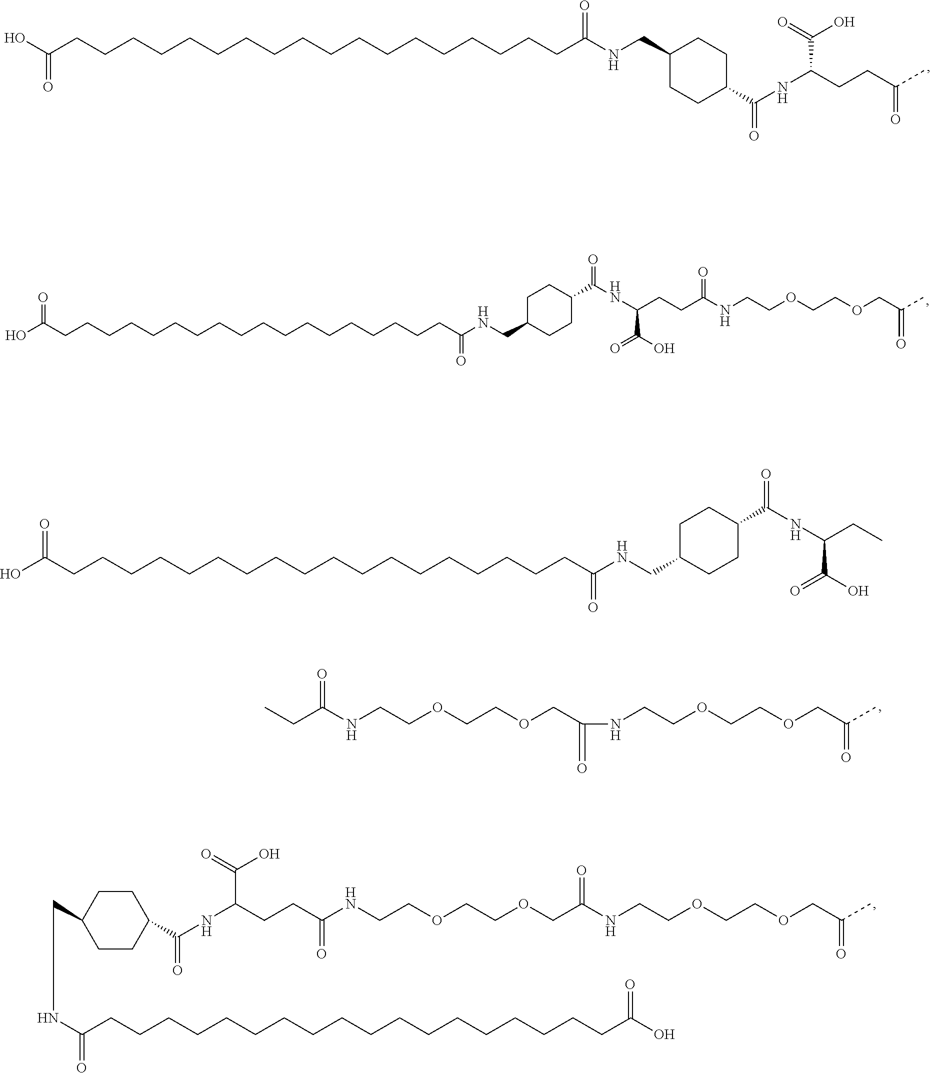

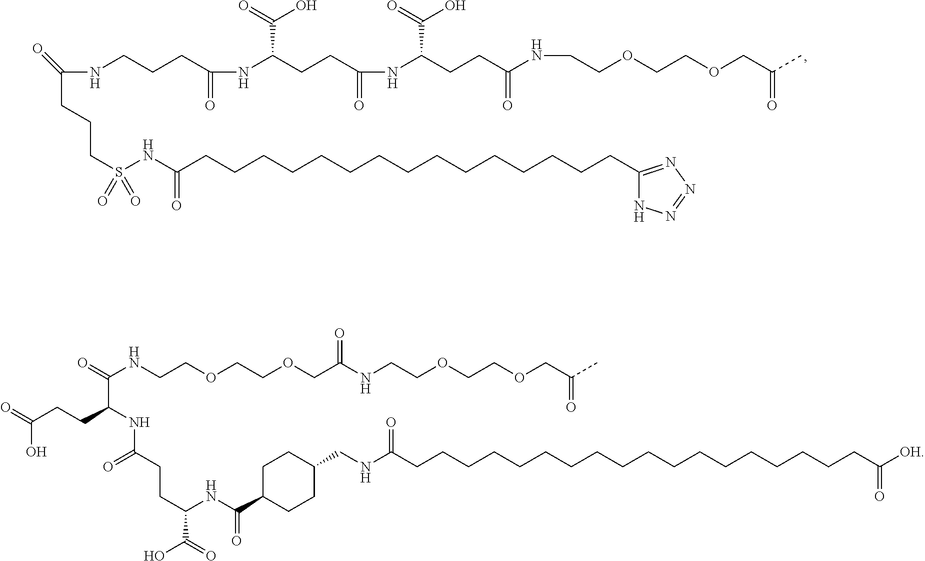

In one embodiment A-B-C-D is composed of an albumin binding fragment A-B-C- and a hydrophilic spacer, D. In one embodiment the serum albumin binding side chain may be linked to an amino, carboxyl, or thiol group, and may be linked by N or C termini, or at the side chains of lysine, aspartic acid, glutamic acid, or cysteine. In one embodiment the serum albumin binding side chain may be linked with diamine and dicarboxylic groups. A range of albumin binding side chains are known among linear and branched lipophilic moieties containing 4-40 carbon atoms having a distal acidic group. In one embodiment the terminal dashed bonds from the attached groups A, B, C and D shown in the formulas herein are to be regarded as attachment bonds and not ending in methylene groups unless stated. In one embodiment the groups A, B, C and/or D are attached to each other by amide bonds.

PYY analogues or derivatives thereof according to the invention (SEQ ID NO: 3 to SEQ ID NO: 30 and SEQ ID NO: 32 to SEQ ID NO: 35) as well as PYY(3-36) (SEQ ID NO: 1) and derivatives hereof (SEQ ID NO: 2 and SEQ ID NO: 31) is shown in Table 1.

TABLE 1
List of PYY analogues or derivatives of the invention and human PYY(3-36)

SEQ ID NO: 1
Name: PYY(3-36)
Structure:

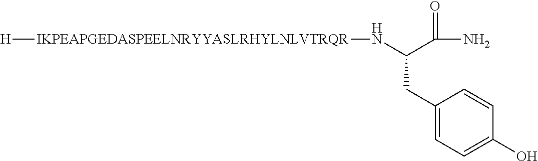

SEQ ID NO: 2
Name: N-alpha-[2-(2-{2-[2-(2-{2-[(S)-4-Carboxy-4-({trans-4-[(19-
carboxynonadecanoylamino)methyl]cyclohexanecarbonyl}amino)butyrylamino]-ethoxy}-
ethoxy)acetylamino]ethoxy}ethoxy)acetyl] PYY(3-36)
Structure:

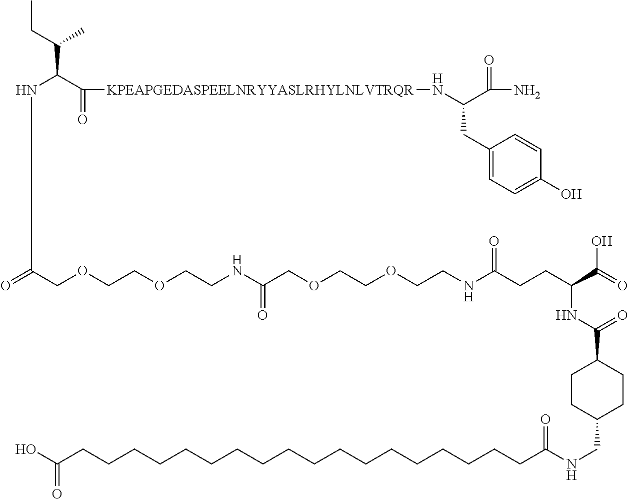

SEQ ID NO: 3
Name: N-alpha-[2-(2-{2-[2-(2-{2-[(S)-4-Carboxy-4-({trans-4-[(19-
carboxynonadecanoylamino)methyl]cyclohexanecarbonyl}amino)butyrylamino]-
ethoxy}ethoxy)acetylamino]ethoxy}ethoxy)acetyl][N-Methyl Gln34] PYY(3-36)
Structure:

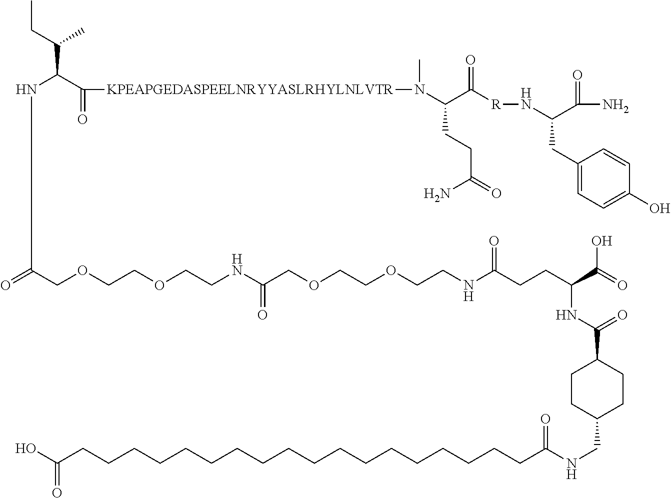

SEQ ID NO: 4
Name: N-alpha-[2-(2-{2-[2-(2-{2-[(S)-4-Carboxy-4-({trans-4-[(19-
carboxynonadecanoylamino)methyl]cyclohexanecarbonyl}amino)butyrylamino]-
ethoxy}ethoxy)acetylamino]ethoxy}ethoxy)acetyl][N-Methyl Arg35] PYY(3-36)
Structure:

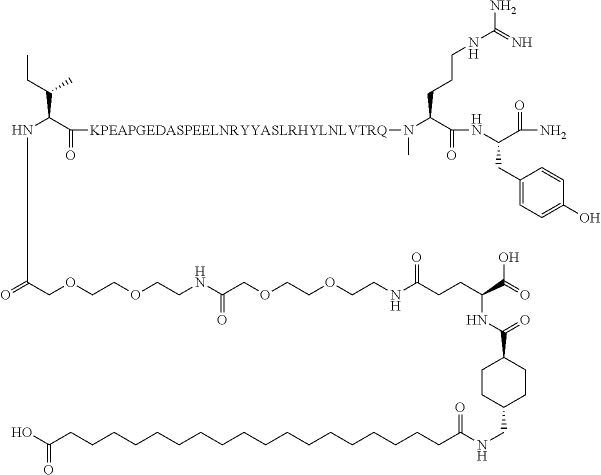

SEQ ID NO: 5
Name: N-alpha-[2-(2-{2-[2-(2-{2-[(S)-4-Carboxy-4-({trans-4-[(19-
carboxynonadecanoylamino)methyl]cyclohexanecarbonyl}amino)butyrylamino]-
ethoxy}ethoxy)acetylamino]ethoxy}ethoxy)acetyl][His25,N-Methyl Gln34] PYY(3-36)
Structure:

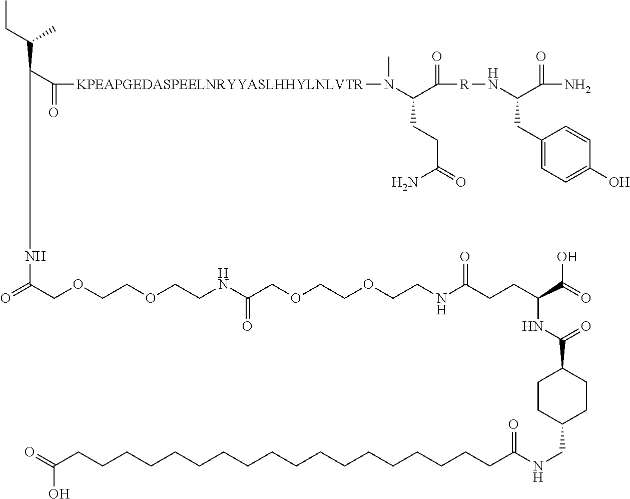

SEQ ID NO: 6
Name: N-alpha-[2-(2-{2-[2-(2-{2-[(S)-4-Carboxy-4-({trans-4-[(19-
carboxynonadecanoylamino)methyl]cyclohexanecarbonyl}amino)butyrylamino]-
ethoxy}ethoxy)acetylamino]ethoxy}ethoxy)acetyl][Aib25,N-Methyl Gln34] PYY(3-36)
Structure:

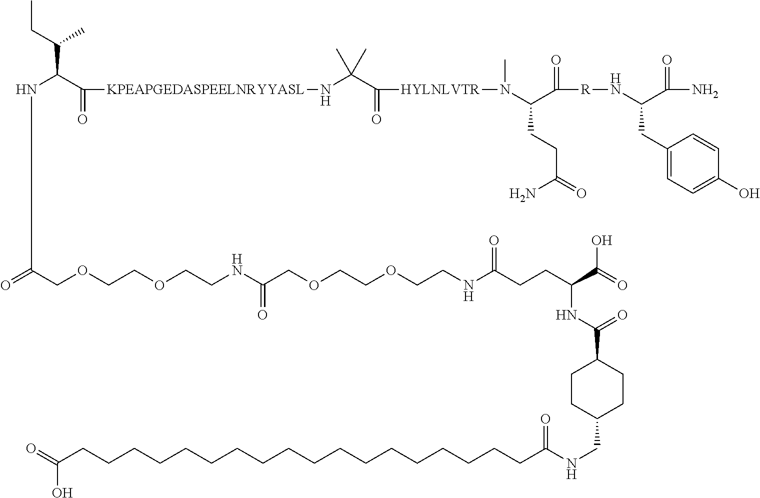

SEQ ID NO: 7
Name: N-alpha-[2-(2-{2-[2-(2-{2-[(S)-4-Carboxy-4-({trans-4-[(19-
carboxynonadecanoylamino)methyl]cyclohexanecarbonyl}amino)butyrylamino]-
ethoxy}ethoxy)acetylamino]ethoxy}ethoxy)acetyl][Tyr25,N-Methyl Gln34] PYY(3-36)
Structure:

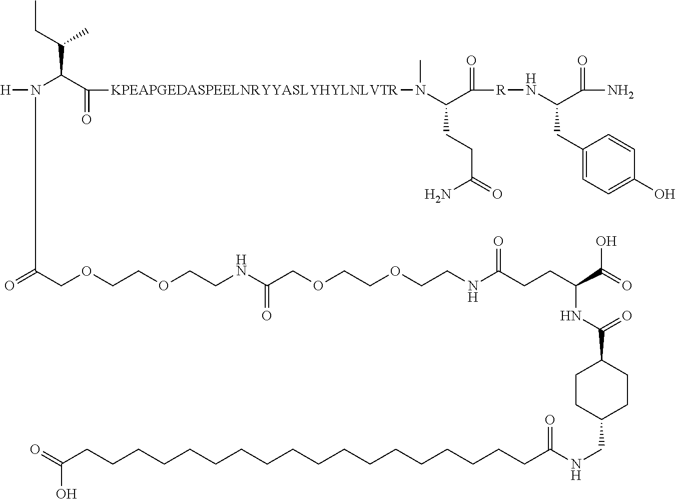

SEQ ID NO: 8
Name: N-alpha-{2-(2-{2-[2-(2-{2-[4-Carboxy-4-(17-
carboxyheptadecanoylamino)butyrylamino]ethoxy}-ethoxy)acetylamino]-
ethoxy}ethoxy)acetyl} [Ala20] Gln34] PYY(3-36)
Structure:

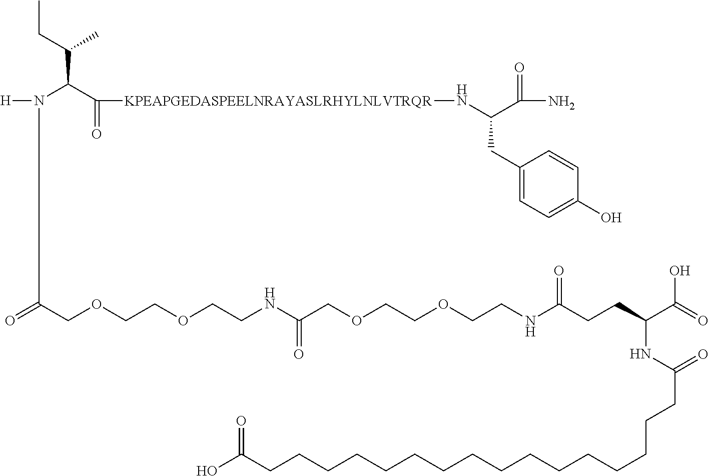

SEQ ID NO: 9
Name: N-alpha-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl} [N-methyl Arg35] PYY(3-36)
Structure:

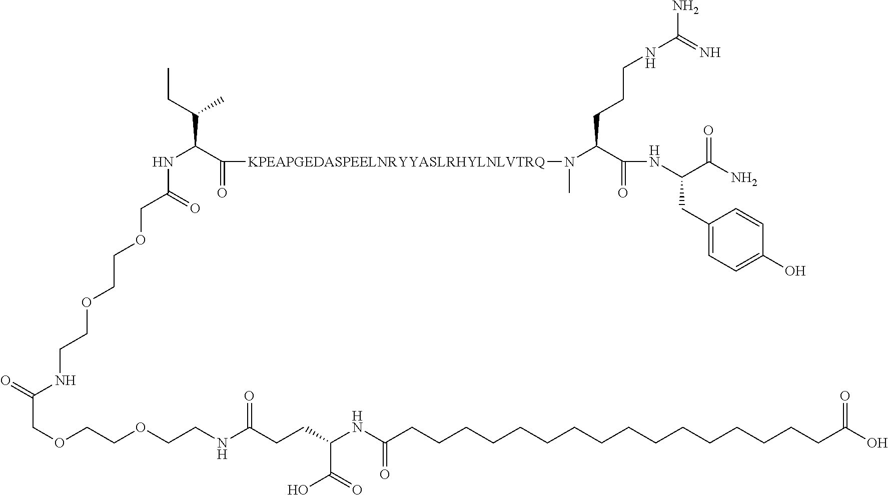

SEQ ID NO: 10
Name: N-alpha-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl} [Ala27] PYY(3-36)
Structure:

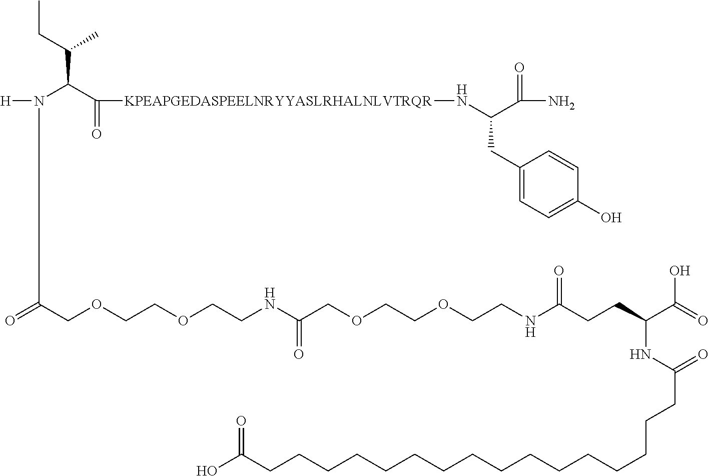

SEQ ID NO: 11
Name: [Cα-methyl Phe36] PYY(3-36)
Structure:

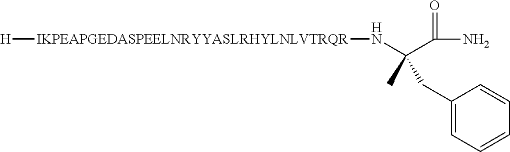

SEQ ID NO: 12
Name: [N-methyl Gln34] PYY(3-36)
Structure:

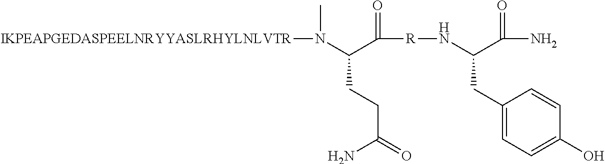

SEQ ID NO: 13
Name: [β-homo Gln34] PYY(3-36)
Structure:

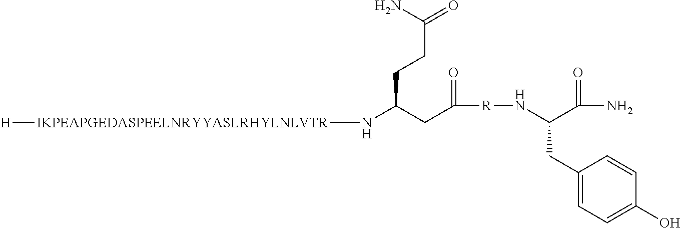

SEQ ID NO: 14
Name: [N-methyl Arg35] PYY(3-36)
Structure:

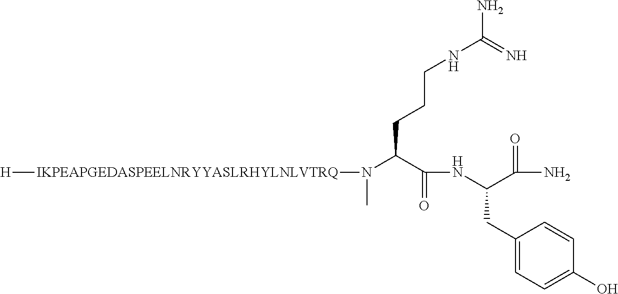

SEQ ID NO: 15
Name: [N-methyl Arg33] PYY(3-36)
Structure:

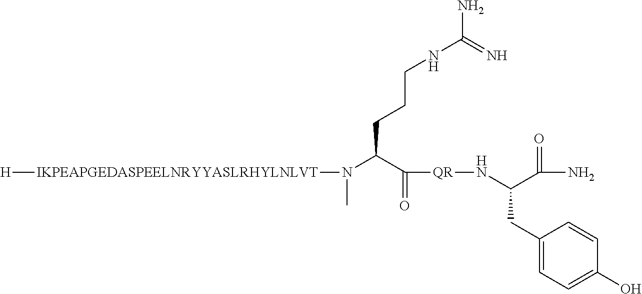

SEQ ID NO: 16
Name: [Agp35] PYY(3-36)
Structure:

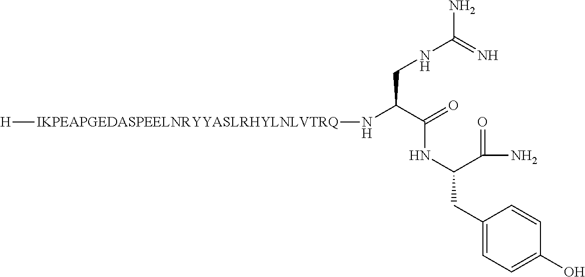

SEQ ID NO: 17
Name: [Agb35] PYY(3-36)
Structure:

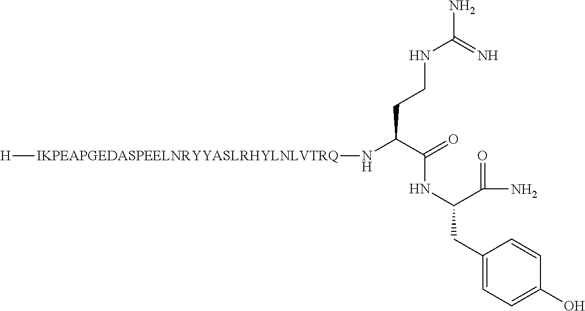

SEQ ID NO: 18
Name: [HomoArg35] PYY(3-36)
Structure:

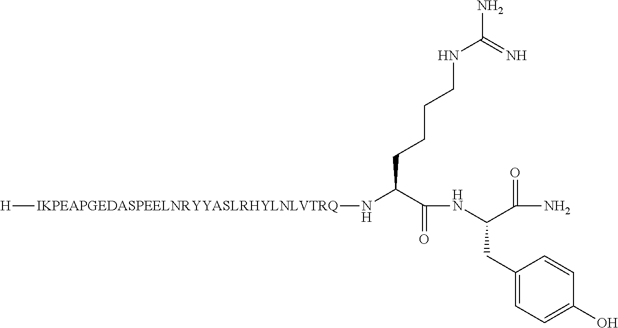

SEQ ID NO: 19
Name: [N-methyl Thr32] PYY(3-36)
Structure:

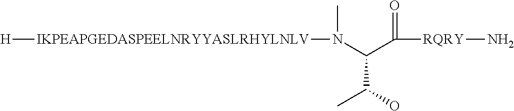

SEQ ID NO: 20
Name: N-alpha-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl} [Agp35] PYY(3-36)
Structure:

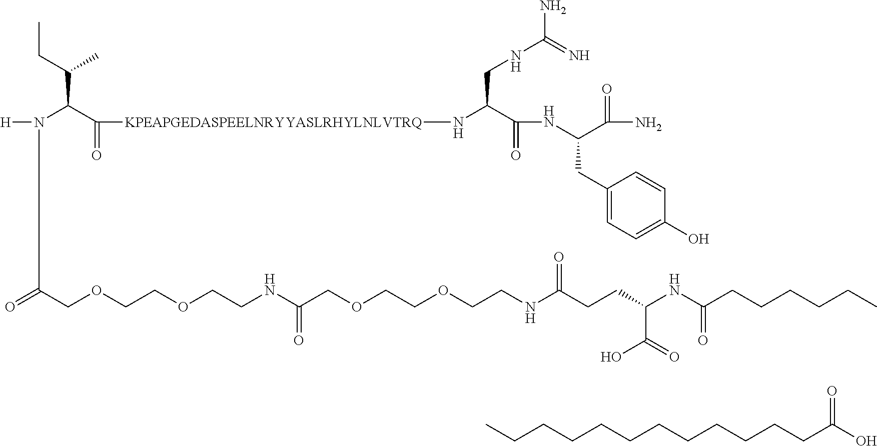

SEQ ID NO: 21
Name: N-alpha-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl} [Agb35] PYY(3-36)
Structure:

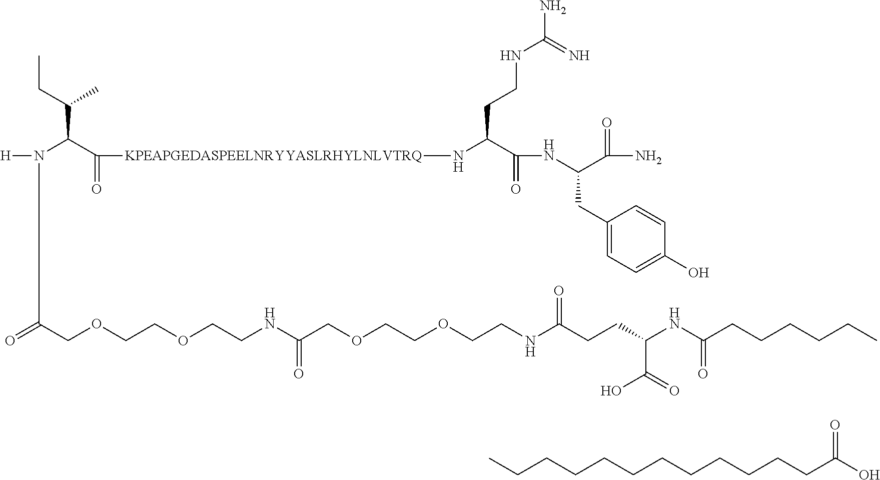

SEQ ID NO: 22
Name: N-alpha-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl} [HomoArg35] PYY(3-36)
Structure:

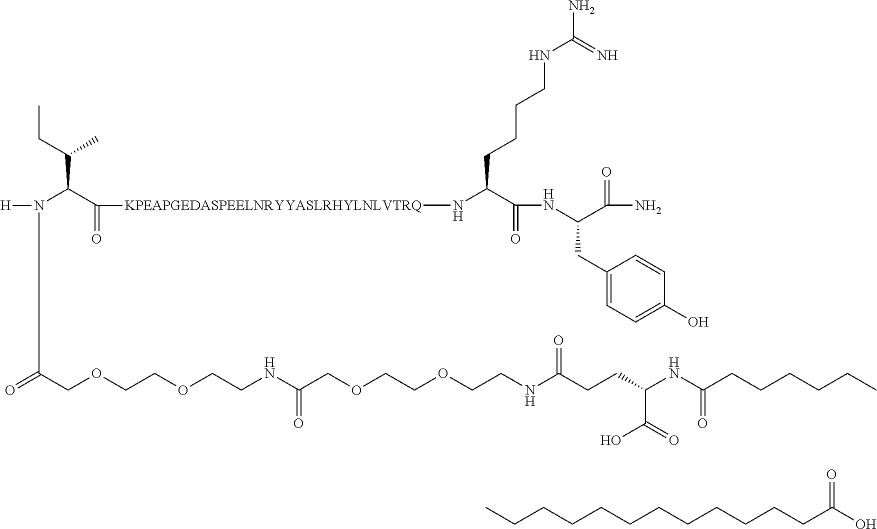

SEQ ID NO: 23
Name: [NArg35] PYY(3-36)
Structure:

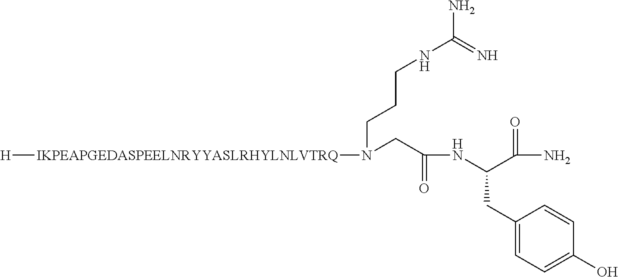

SEQ ID NO: 24
Name: [NGln34] PYY(3-36)
Structure:

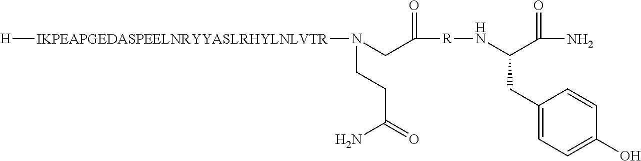

SEQ ID NO: 25
Name: N-alpha-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl} [NArg35] PYY(3-36)
Structure:

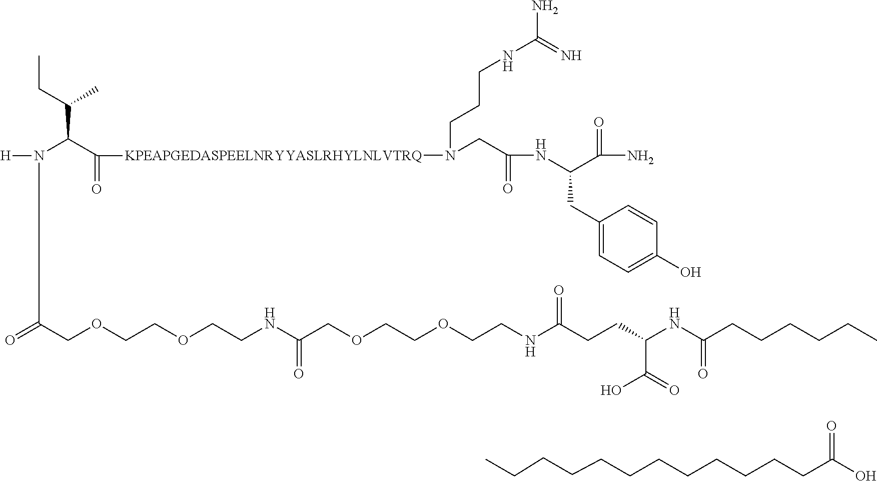

SEQ ID NO: 26
Name: N-alpha-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl} [NGln34] PYY(3-36)
Structure:

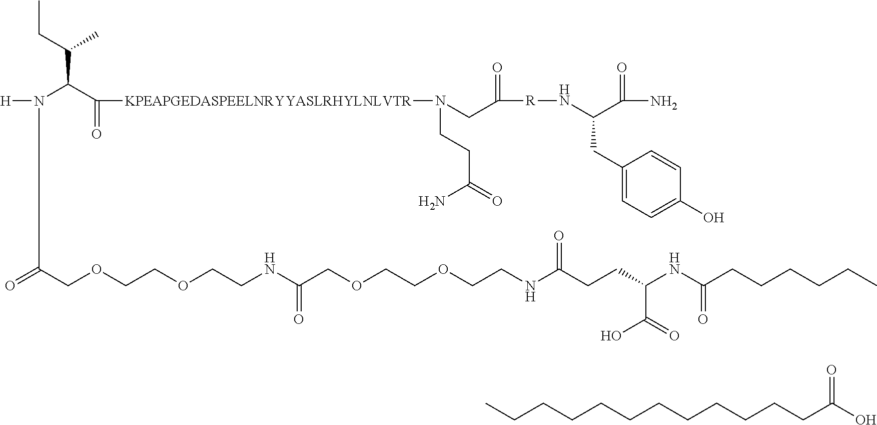

SEQ ID NO: 27
Name: [N-methyl Tyr36] PYY(3-36)
Structure:

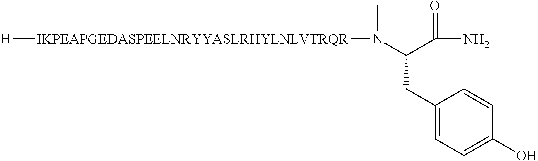

SEQ ID NO: 28
Name: [Cα-methyl Tyr36] PYY(3-36)
Structure:

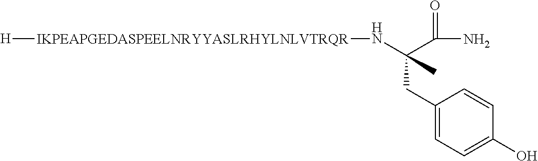

SEQ ID NO: 29
Name: N-epsilon13-[2-(2-{2-[2-(2-{2-[(S)-4-Carboxy-4-({trans-4-[(19-
carboxynonadecanoylamino)methyl]cyclohexanecarbonyl}amino)butyrylamino]-
ethoxy}ethoxy)acetylamino]ethoxy}ethoxy)acetyl][Lys13,N-methyl Tyr36] PYY(3-36)
Structure:

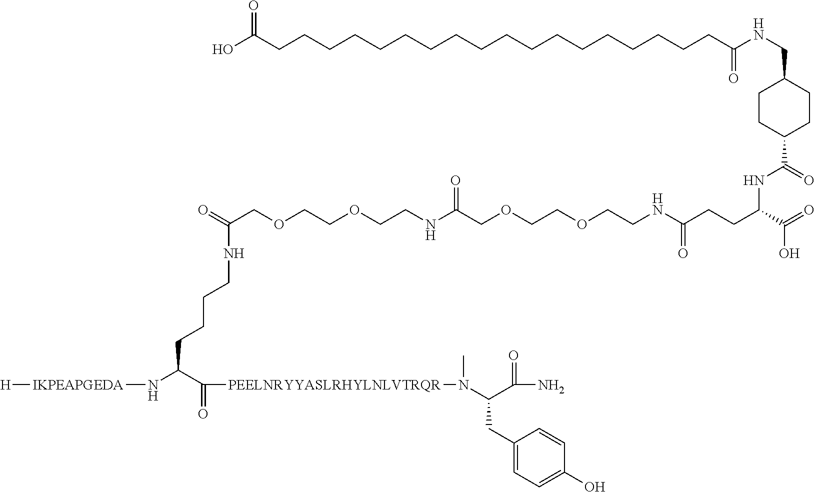

SEQ ID NO: 30
Name: N-alpha-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl} [Cα-methyl Tyr36] PYY(3-36)
Structure:

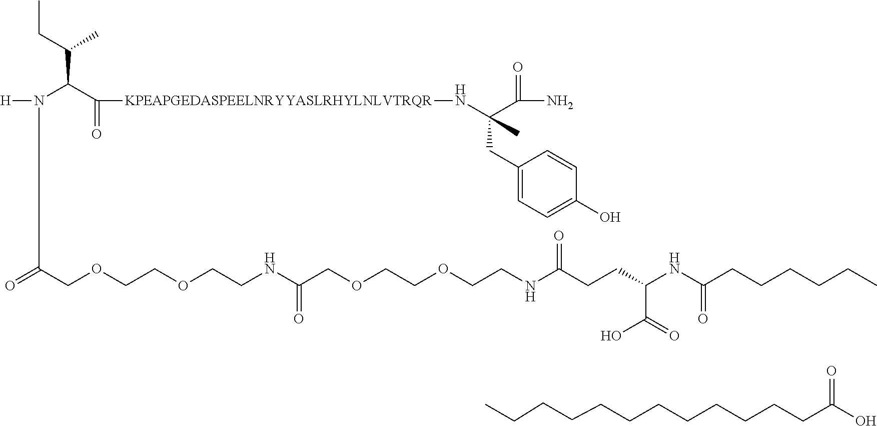

SEQ ID NO: 31
Name: [N-alpha-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl}] PYY(3-36)
Structure:

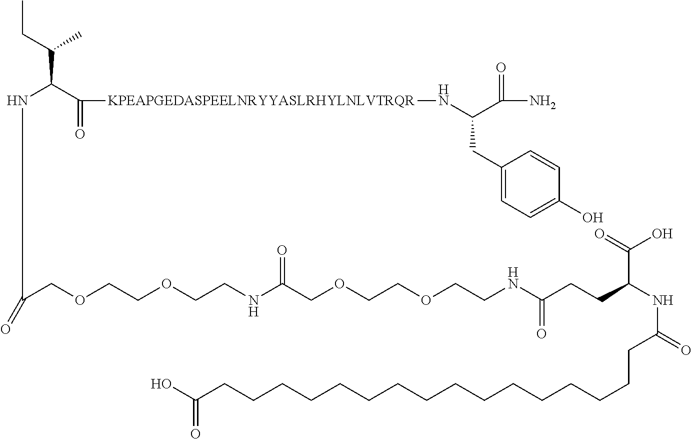

SEQ ID NO: 32
Name: [N-alpha-acetyl, N-methyl Arg35] PYY(3-36)
Structure:

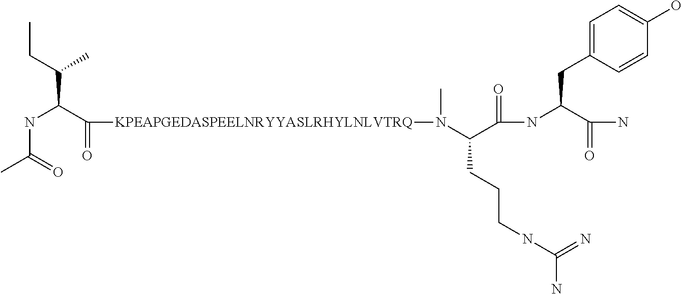

SEQ ID NO: 33
Name: [N-alpha-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl}[NTyr36]]PYY3-36
Structure:

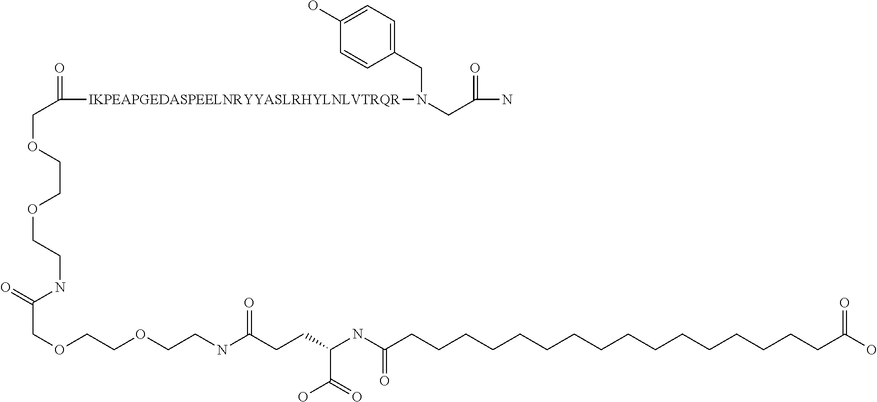

SEQ ID NO: 34
Name: [NTyr36]PYY3-36
Structure:

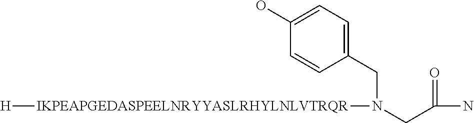

SEQ ID NO: 35
Name: N-alpha-Acetyl[N-epsilon10-{2-(2-{2-[2-(2-{2-[4-Carboxy-4-(17-
carboxyheptadecanoylamino)butyrylamino]ethoxy}ethoxy)-acetylamino]-
ethoxy}ethoxy)acetyl}][Lys10,N-methyl Arg35]] PYY(3-36)
Structure:

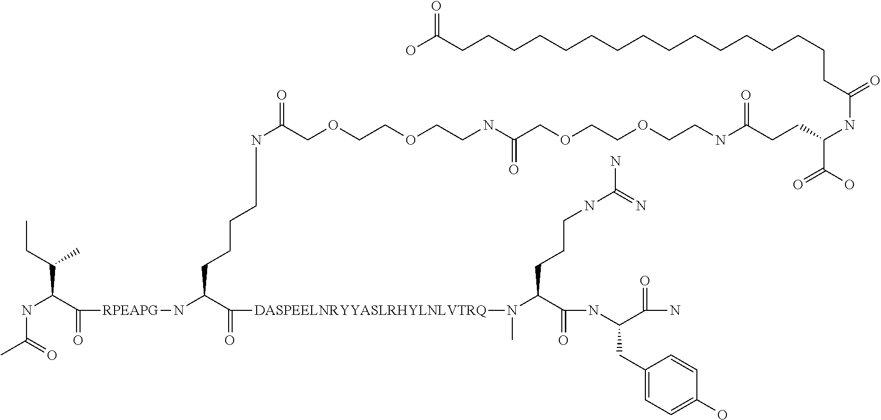

In one embodiment the PYY analogue or a derivative thereof is SEQ ID NO: 3. In one embodiment the PYY analogue or a derivative thereof is SEQ ID NO: 4. In one embodiment the PYY analogue or a derivative thereof is SEQ ID NO: 5. In one embodiment the PYY analogue or a derivative thereof is SEQ ID NO: 6. In one embodiment the PYY analogue or a derivative thereof is SEQ ID NO: 7. In one embodiment the PYY analogue or a derivative thereof is SEQ ID NO: 8. In one embodiment the PYY analogue or a derivative thereof is SEQ ID NO: 9. In one embodiment the PYY analogue or a derivative thereof is SEQ ID NO: 10. In one embodiment the PYY analogue or a derivative thereof is SEQ ID NO: 20. In one embodiment the PYY analogue or a derivative thereof is SEQ ID NO: 21. In one embodiment the PYY analogue or a derivative thereof is SEQ ID NO: 22.

In one embodiment the PYY analogue or a derivative thereof is SEQ ID NO: 25. In one embodiment the PYY analogue or a derivative thereof is SEQ ID NO: 26. In one embodiment the PYY analogue or a derivative thereof is SEQ ID NO: 29. In one embodiment the PYY analogue or a derivative thereof is SEQ ID NO: 30. In one embodiment the PYY analogue or a derivative thereof is SEQ ID NO: 33. In one embodiment the PYY analogue or a derivative thereof is SEQ ID NO: 35.

In one embodiment the PYY analogue or a derivative thereof is SEQ ID NO: 11. In one embodiment the PYY analogue or a derivative thereof is SEQ ID NO: 13. In one embodiment the PYY analogue or a derivative thereof is SEQ ID NO: 16. In one embodiment the PYY analogue or a derivative thereof is SEQ ID NO: 17. In one embodiment the PYY analogue or a derivative thereof is SEQ ID NO: 18. In one embodiment the PYY analogue or a derivative thereof is SEQ ID NO: 19. In one embodiment the PYY analogue or a derivative thereof is SEQ ID NO: 23. In one embodiment the PYY analogue or a derivative thereof is SEQ ID NO: 24. In one embodiment the PYY analogue or a derivative thereof is SEQ ID NO: 28. In one embodiment the PYY analogue or a derivative thereof is SEQ ID NO: 32. In one embodiment the PYY analogue or a derivative thereof is SEQ ID NO: 34.

In one embodiment the PYY analogue or derivative thereof retain at least about 25%, specifically about 30%, 40%, 50%, 60%, 70%, 80%, 90%, 95%, 98%, or 99% percent of the biological activity of human PYY(1-36). In one embodiment the term “biological activity” of PYY is intended to mean the ability to induce an effect in an in vivo model such as the assay for acute food-intake in mice described herein. In one embodiment “biological activity” of PYY is intended to mean reduction of food intake, effect on body weight, gastric emptying, change in respiratory quotient and/or effect on intestinal electrolyte secretion. Methods for determination of said biological effects are described herein, e.g., in Assay (V), Assay (VII), Assay (VI), Assay (VIII) and Assay (IX). In one embodiment the PYY analogue or derivative thereof exhibit improved biological activity compared to human PYY(1-36). In one embodiment the PYY analogue or derivative thereof exhibit at least about 110%, 125%, 130%, 140%, 150%, 200% or more of the biological activity of human PYY(1-36) or human PYY(3-36). In one embodiment the PYY analogue or derivative thereof has an effect in at least one of the assays described herein, such as food intake, effect on body weight, gastric emptying, change in respiratory quotient, effect on intestinal electrolyte secretion, Assay (V), Assay (VII), Assay (VI), Assay (VIII) and Assay (IX), which is equal to or greater than the potency of human PYY(1-36) or human PYY(3-36) in the same assay. In one embodiment the PYY analogue or derivative thereof exhibits improved ease of manufacture, stability and/or ease of formulation compared to human PYY(1-36) or human PYY(3-36).

In one embodiment the PYY analogue or derivative thereof has improved pharmacokinetic profile compared to human PYY(1-36), human PYY(3-36) or human PYY(3-36) with a serum albumin binding side chain identical to that of said derivative. In one embodiment the PYY analogue or derivative thereof comprising a serum albumin binding side chain according to the invention displays protracted properties that make them suitable administration once daily or with lower frequency than once-daily, such as in a once-weekly, twice-monthly, or once-monthly dosing regime. In one embodiment said pharmacokinetic profile or said protracted properties is determined by measuring the half life of the PYY analogue or derivative thereof.

In one embodiment the invention provides the PYY analogue or derivative thereof with high affinity albumin binding effect. In one embodiment high affinity albumin binding effect is defined as at least 10 times, such as at least 20 times, at least 50 times, or at least 100 times higher albumin binding of the PYY analogue or derivative thereof according to the invention relative to human PYY(1-36), human PYY(3-36) or human PYY(3-36) with a serum albumin binding side chain identical to that of said analogue or derivative.

In one embodiment the PYY analogue or derivative thereof has substantially improved half life relative to human PYY(1-36) or human PYY(3-36). In one embodiment the half life of the PYY analogue or derivative thereof in a rodent or in a non-rodent model is improved at least 3 fold, such as at least 6 fold, 10 fold or 50 fold, relative to human PYY(1-36) or human PYY(3-36). In one embodiment the PYY analogue or derivative thereof shows an improvement of half life compared to human PYY(3-36) in the range of 5-500, such as 10-500, 20-500, 50-500, 10-400, 20-400, 50-400, 100-500, 100-400 or 200-500 fold determined in vivo using a non-rodent model. In one embodiment the PYY analogue or derivative thereof shows an improvement of half life compared to human PYY(3-36) in the range of 1.2-4 fold determined in vivo using a non-rodent model. In one embodiment the PYY analogue or derivative thereof has a substantially improved half life in a non-rodent model relative to human PYY(1-36) or human PYY(3-36), wherein said analogue or derivative has at least the same level of potency for activation of the Y2 receptor as human PYY(1-36) or human PYY(3-36). In one embodiment the PYY analogue or derivative thereof has substantially improved half life in a non-rodent model relative to human PYY(1-36) or human PYY(3-36), wherein said analogue or derivative has at least 50%, such as 60%, 70%, 80% or 80% potency for activation of the Y2 receptor as human PYY(1-36) or human PYY(3-36). In one embodiment half life or half life is determination relative to human PYY(1-36), human PYY(3-36) or human PYY(3-36) with a serum albumin binding side chain identical to that of said analogue or derivative.

In one embodiment the PYY analogue or derivative thereof has an in vivo half life of at least 10 h, such as at least 20 h, at least 30 h, at least 40 h, at least 50 h, at least 100 h, at least 150 h, at least 200 h, at least 250 h, at least 300 h, or at least 350 h after i.v. administration to mini pigs, and alternatively an in vivo half life of at least 80 h after i.v. administration to mini pigs.

In one embodiment the half life of the PYY analogue or derivative thereof is longer than the half life of human PYY(3-36). In one embodiment the half life of the PYY analogue or derivative thereof is at least 1.1, such as at least 1.2, at least 1.3, at least 1.4 or at least 1.5 times the half life of human PYY(3-36). In one embodiment the half life of the PYY analogue or derivative thereof is at least 2, at least 4, at least 6, at least 8 or at least 10 times the half life of human PYY(3-36). In one embodiment the half life of the PYY analogue or derivative thereof is at least 0.8, at least 0.85, at least 0.90%, at least 0.95% or 1 times the half life of human PYY(3-36). In one embodiment half life is determined by Assay (IV) as described herein, in vitro half life in plasma.

In one embodiment the half life of the PYY analogue or derivative thereof is longer than the half life of human PYY(3-36). In one embodiment the half life of the PYY analogue or derivative thereof is at least 10 times, such as at least 20, at least 40, at least 60, at least 75, at least 100, at least 150, at least 200, at least 250, at least 300, at least 350 or at least 400 times the half life of human PYY(3-36). In one embodiment the half life of the PYY derivative according to the invention is longer than the half life of human PYY(3-36) with a serum albumin binding side chain identical to that of said analogue or derivative. In one embodiment the half life of a PYY derivative according to the invention is at least 2 times, such as at least 3, at least 4, at least 5, at least 6, at least 7, at least 8, at least 9, at least 10, at least 12, at least 15 or at least 20 times the half life of human PYY(3-36) with a serum albumin binding side chain identical to that of said analogue or derivative. In one embodiment half life is determined by Assay (V) as described herein, PK i.v. minipigs.

In one embodiment the PYY analogue or derivative thereof comprises at least one serum albumin binding side chain and at least one alteration selected from the group consisting of substitutions, insertions, deletions and modifications that stabilize against proteolytic breakdown, wherein said serum albumin binding side chain and said at least one alteration act synergistically to prolong the half life of said analogue or derivative in vivo compared to human PYY(3-36). The term “proteolytic breakdown” is intended to mean the degradation of a peptide by proteases and peptidases in vivo or in vitro. In one ascpect the term “stability against proteolytic breakdown” is intended to mean that a peptide in resistant to degradation by proteases and peptidases. Proteolytic breakdown as well as stability against proteolytic breakdown may be determined using Assay (I) as described herein.

In one embodiment the Y2 receptor potency of the PYY analogue or derivative is less than 2 times the Y2 receptor potency of human PYY(3-36). In one embodiment the Y2 receptor potency of the PYY analogue or derivative is less than 5 times, such as less than 10, 20, 50, 100 or 150 times the Y2 receptor potency of human PYY(3-36). In one embodiment the Y2 receptor potency of the PYY analogue or derivative is less than the Y2 receptor potency of human PYY(3-36). In one embodiment the Y2 receptor potency of the PYY analogue or derivative is between 0.5 and 5 times the Y2 receptor potency of human PYY(3-36). In one embodiment the Y2 receptor potency is determined by Assay (I), Y2 receptor ACTOne assay. In one embodiment a lower receptor potency corresponds to a higher EC50 value.

In one embodiment the PYY analogue or derivative thereof has a Y1 receptor potency which is higher than the Y1 receptor potency of hPYY(3-36) as determined by Assay (II).

In one embodiment the PYY analogue or derivative thereof has a Y5 receptor potency which is higher than the Y5 receptor potency of hPYY(3-36) as determined by Assay (III).

In one embodiment the PYY analogue or derivative thereof has a Y5/Y2 receptor potency ratio which is at least 5 as determined by Assay (III) and Assay (I), respectively. In one embodiment the PYY analogue or derivative thereof has a Y5/Y2 receptor potency ratio which is at least equal to or higher than the Y5/Y2 receptor potency ratio of hPYY(3-36) as determined by Assay (III) and Assay (I), respectively. In one embodiment the PYY analogue or derivative thereof has a Y5/Y2 receptor potency ratio which at least 15 or at least 20 as determined by Assay (III) and Assay (I), respectively.

In one embodiment the PYY analogue or derivative thereof has a Y1/Y2 receptor potency ratio which is at least 2 as determined by Assay (II) and Assay (I), respectively. In one embodiment the PYY analogue or derivative thereof has a Y1/Y2 receptor potency ratio which is higher than the Y1/Y2 receptor potency ratio of hPYY(3-36) as determined by Assay (II) and Assay (I), respectively. In one embodiment the PYY analogue or derivative thereof has a Y1/Y2 receptor potency ratio which is at least 15 or at least 20 as determined by Assay (II) and Assay (I), respectively. In one embodiment the PYY analogue or derivative thereof has a Y1/Y2 receptor potency ratio which is at least 30 or at least 50 as determined by Assay (II) and Assay (I), respectively.

In one embodiment the PYY analogue or derivative thereof has a half-life of at least 8 hours as determined by Assay (IV) and a Y2 receptor potency of less than 10 nM as determined by Assay (I). In one embodiment the PYY analogue or derivative thereof has a half-life of at least 8 hours as determined by Assay (IV), a Y2 receptor potency of less than 10 nM as determined by Assay (I), and a Y5/Y2 receptor potency ratio of at least 5 as determined by Assay (III) and Assay (I), respectively. In one embodiment the PYY analogue or derivative thereof has a half-life of at least 8 hours as determined by Assay (IV), a Y2 receptor potency of less than 10 nM as determined by Assay (I), and a Y1/Y2 receptor potency ratio of at least 2 as determined by Assay (III) and Assay (I), respectively.

Y2 receptor selectivity is intended to mean the ability to selectively activate the Y2 receptor relative to the Y1 and/or the Y5 receptor. The selectivity for the Y2 receptor relative to the Y1 or Y5 receptor is determined by the ratio of Y1/Y2 potency or Y5/Y2 potency ratio, respectively. The Y2, Y1, and Y5 receptor potency is determined by Assay (I), Assay (II), and Assay (III), respectively.

In one embodiment effects of the PYY analogue or derivative thereof described herein is determined relative to hPYY(3-36) with a serum albumin binding side chain identical to that of said derivative.

### Pharmaceutical Compositions

The present invention also relates to a pharmaceutical composition comprising a peptide or a derivative of the invention; or a pharmaceutically acceptable salt, amide, or ester thereof, and a pharmaceutically acceptable excipient.

One object of the present invention is to provide a pharmaceutical formulation comprising the PYY analogue or derivative thereof which is present in a concentration from 0.1 mg/ml to 25 mg/ml, and wherein said formulation has a pH from 3.0 to 9.0. The formulation may further comprise at least one selected from the group consisting of a buffer system, preservative(s), tonicity agent(s), chelating agent(s), stabilizer(s) and surfactant(s). The term “pharmaceutical composition” as used herein means a product comprising an active analogue or derivative according to the invention together with pharmaceutical excipients selecting from the group cinsisting of a buffer, a preservative, and optionally a tonicity modifier and/or a stabilizer. The PYY analogues or derivatives thereof and compositions containing them are also useful in the manufacture of a medicament for therapeutic applications mentioned herein.

The term “excipient” broadly refers to any component other than the active therapeutic ingredient(s). In a particular embodiment the excipient is an inert substance. In another embodiment it is an inactive substance. In a still further embodiment it is a not medicinally active substance.

The excipient may serve various purposes, e.g. as a carrier, vehicle, diluent, tablet aid, and/or to improve administration, and/or absorption of the active substance.

The formulation of pharmaceutically active ingredients with various excipients is known in the art, see e.g. Remington: The Science and Practice of Pharmacy (e.g. 19^{th} edition (1995), and any later editions).

Non-limiting examples of excipients for potential use in the composition of the invention are: Solvents, diluents, buffers, preservatives, tonicity regulating agents, chelating agents, and stabilisers. Any one or more of these may be incorporated in the pharmaceutical composition of the invention.

In one embodiment of the invention, the pharmaceutical composition is a liquid formulation. A preferred example of a liquid formulation is an aqueous formulation, i.e. a formulation comprising water. The liquid formulation may be a solution, or a suspension. An aqueous formulation typically comprises at least 50% w/w water, or at least 60%, 70%, 80%, or even at least 90% w/w of water.

In another embodiment, the pharmaceutical composition is a solid formulation, e.g. a freeze-dried or spray-dried composition, which may be used as is, or whereto the physician or the patient adds solvents, and/or diluents prior to use.

The pH in an aqueous formulation may be anything between pH 3 and pH 10. In one embodiment of the invention, the pH of the formulation is from about 7.0 to about 9.5. In another embodiment of the invention, the pH of the formulation is from about 3.0 to about 7.0.

In a further embodiment of the invention, the pharmaceutical composition comprises a buffer. The buffer may e.g. be selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid, and mixtures thereof. Each one of these specific buffers constitutes an alternative embodiment of the invention.

In a further embodiment of the invention, the pharmaceutical composition comprises a preservative. The preservative may e.g. be selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol), and mixtures thereof. The preservative may be present in a concentration from 0.1 mg/ml to 20 mg/ml. Each one of these specific preservatives constitutes an alternative embodiment of the invention.

In a further embodiment of the invention, the pharmaceutical composition comprises an isotonic agent. The isotonic agent may e.g. be selected from the group consisting of a salt (e.g. sodium chloride), a sugar or sugar alcohol, an amino acid (e.g. glycine, histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), and mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alfa and beta HPCD, soluble starch, hydroxyethyl starch and carboxymethylcellulose-Na may be used. Sugar alcohol is defined as a C4-C8 hydrocarbon having at least one —OH group and includes, for example, mannitol, sorbitol, inositol, galactitol, dulcitol, xylitol, and arabitol. In one embodiment, the sugar alcohol additive is mannitol. Each one of these specific isotonic agents constitutes an alternative embodiment of the invention.

In a further embodiment of the invention, the pharmaceutical composition comprises a chelating agent. The chelating agent may e.g. be selected from salts of ethylenediaminetetraacetic acid (EDTA), citric acid, and aspartic acid, and mixtures thereof. Each one of these specific chelating agents constitutes an alternative embodiment of the invention.

In a further embodiment of the invention, the pharmaceutical composition comprises a stabiliser. The stabiliser may e.g. be one or more oxidation inhibitors, aggregation inhibitors, surfactants, and/or one or more protease inhibitors. Non-limiting examples of these various kinds of stabilisers are disclosed in the following.

The term “aggregate formation” refers to a physical interaction between the peptide molecules resulting in formation of oligomers, which may remain soluble, or large visible aggregates that precipitate from the solution. Aggregate formation by a peptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical composition of the invention may comprise an amount of an amino acid base sufficient to decrease aggregate formation of the polypeptide during storage of the composition. The term “amino acid base” refers to one or more amino acids (such as methionine, histidine, imidazole, arginine, lysine, isoleucine, aspartic acid, tryptophan, threonine), or analogues thereof. Any amino acid may be present either in its free base form or in its salt form. Any stereoisomer (i.e., L, D, or a mixture thereof) of the amino acid base may be present.

In a further embodiment of the invention, methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. Any stereoisomer of methionine (L or D) or combinations thereof can be used.

In a further embodiment of the invention, the pharmaceutical composition comprises a stabiliser selected from the group of high molecular weight polymers or low molecular compounds. The stabiliser may e.g. be selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilisers constitutes an alternative embodiment of the invention.

The pharmaceutical composition of the invention may comprise additional stabilising agents such as, but not limited to, methionine and EDTA, which protect the polypeptide against methionine oxidation, and a nonionic surfactant, which protects the polypeptide against aggregation associated with freeze-thawing or mechanical shearing.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant may e.g. be selected from the group consisting of anionic surfactants, cationic surfactants, nonionic surfactants, and/or zwitterionic surfactants.

In a further embodiment of the invention, the pharmaceutical composition comprises one or more protease inhibitors, such as, e.g., EDTA (ethylenediamine tetraacetic acid), and/or benzamidineHCl.

Additional, optional, ingredients of the pharmaceutical composition of the invention include, e.g., wetting agents, emulsifiers, antioxidants, bulking agents, metal ions, oily vehicles, proteins (e.g., human serum albumin, gelatine), and/or a zwitterion (e.g., an amino acid such as betaine, taurine, arginine, glycine, lysine and histidine).

In an embodiment of the invention, each administered dose contains from 0.01 mg-10 mg of the derivative.

The derivative of the invention is preferably administered in the form of a pharmaceutical composition. It may be administered to a patient in need thereof at several sites, for example, at topical sites such as skin or mucosal sites; at sites which bypass absorption such as in an artery, in a vein, or in the heart; and at sites which involve absorption, such as in the skin, under the skin, in a muscle, or in the abdomen.

The route of administration may be, for example, lingual; sublingual; buccal; in the mouth; oral; in the stomach; in the intestine; nasal; pulmonary, such as through the bronchioles, the alveoli, or a combination thereof; parenteral, epidermal; dermal; transdermal; conjunctival; uretal; vaginal; rectal; and/or ocular. In a particular embodiment the composition is an oral composition, and the route of administration is per oral.

The composition of the invention may be administered in several dosage forms, for example as a solution; a suspension; an emulsion; a microemulsion; multiple emulsions; a foam; a salve; a paste; a plaster; an ointment; a tablet; a coated tablet; a chewing gum; a rinse; a capsule such as hard or soft gelatine capsules; a suppositorium; a rectal capsule; drops; a gel; a spray; a powder; an aerosol; an inhalant; eye drops; an ophthalmic ointment; an ophthalmic rinse; a vaginal pessary; a vaginal ring; a vaginal ointment; an injection solution; an in situ transforming solution such as in situ gelling, setting, precipitating, and in situ crystallisation; an infusion solution; or as an implant. In a particular embodiment the composition of the invention is a tablet, optionally coated, a capsule, or a chewing gum.

The composition of the invention may further be compounded in a drug carrier or drug delivery system, e.g. in order to improve stability, bioavailability, and/or solubility. In a particular embodiment the composition of the invention may be attached to such system through covalent, hydrophobic, and/or electrostatic interactions. The purpose of such compounding may be, e.g., to decrease adverse effects, achieve chronotherapy, and/or increase patient compliance.

The composition of the invention may also be used in the formulation of controlled, sustained, protracting, retarded, and/or slow release drug delivery systems.

Parenteral administration may be performed by subcutaneous, intramuscular, intraperitoneal, or intravenous injection by means of a syringe, optionally a pen-like syringe, or by means of an infusion pump.

The composition of the invention may be administered nasally in the form of a solution, a suspension, or a powder; or it may be administered pulmonally in the form of a liquid or powder spray.

Transdermal administration is a still further option, e.g. by needle-free injection, from a patch such as an iontophoretic patch, or via a transmucosal route, e.g. buccally.

In a particular embodiment the composition of the invention is a stabilised formulation. The term “stabilised formulation” refers to a formulation with increased physical and/or chemical stability, preferably both. In general, a formulation must be stable during use and storage (in compliance with recommended use and storage conditions) until the expiration date is reached.

The term “physical stability” refers to the tendency of the polypeptide to form biologically inactive and/or insoluble aggregates as a result of exposure to thermomechanical stress, and/or interaction with destabilising interfaces and surfaces (such as hydrophobic surfaces). The physical stability of an aqueous polypeptide formulation may be evaluated by means of visual inspection, and/or by turbidity measurements after exposure to mechanical/physical stress (e.g. agitation) at different temperatures for various time periods. Alternatively, the physical stability may be evaluated using a spectroscopic agent or probe of the conformational status of the polypeptide such as e.g. Thioflavin T or “hydrophobic patch” probes.

The term “chemical stability” refers to chemical (in particular covalent) changes in the polypeptide structure leading to formation of chemical degradation products potentially having a reduced biological potency, and/or increased immunogenic effect as compared to the intact polypeptide. The chemical stability can be evaluated by measuring the amount of chemical degradation products at various time-points after exposure to different environmental conditions, e.g. by SEC-HPLC, and/or RP-HPLC.

The treatment with a derivative according to this invention may also be combined with a surgery that influences the glucose levels, and/or lipid homeostasis such as gastric banding or gastric bypass.

It should be understood that any suitable combination of the derivatives according to the invention with one or more of the above-mentioned compounds and optionally one or more further pharmacologically active substances are considered to be within the scope of the present invention.

### Clinical Indications

In one embodiment the invention relates to the use of at least one PYY analogue or derivative thereof for the preparation of a medicament. In one embodiment a method of treating a disease, condition or disorder modulated by a Y2 receptor agonist using the PYY analogue or derivative thereof is provided. In one embodiment the PYY analogue or derivative thereof is administered peripherally, such as i.v, s.c. or orally. In one embodiment the subject to be treated by a method of the invention is a mammal, such as a human, a cat or a dog. In one embodiment a therapeutically effective amount of the PYY analogue or derivative thereof is used. The PYY analogue or derivative thereof may be used alone or in combination with at least one additional pharmaceutical agent that is useful in the treatment of the disease, condition or disorder or a co-morbidity of the disease, condition or disorder. Diseases, conditions, or disorders modulated by a Y2 receptor agonist in mammals include obesity and being overweight. Co-morbidities of such diseases, conditions, or disorders would likely be incidentally improved by treatment of such diseases, conditions, or disorders. In one embodiment a method of treating obesity using the PYY analogue or derivative thereof is provided.

As used herein, the term “therapeutically effective amount” of a compound refers to an amount sufficient to cure, alleviate, or partially arrest the clinical manifestations of a given disease and/or its complications with respect to appropriate control values determined prior to treatment or in a vehicle-treated group. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. In one embodiment the terms are intended to include the full spectrum of treatments for a given condition from which the patient is suffering, such as administration of the PYY analogue or derivative thereof in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the PYY analogue or derivative thereof in question to prevent the onset of symptoms or complications. The terms “treating”, “treat”, or “treatment” embrace both preventative, i.e., prophylactic, and palliative treatment. In one embodiment the invention relates to a method of reducing weight or promoting weight loss (including preventing or inhibiting weight gain) in a mammal which comprises peripherally administering to the mammal a weight-controlling or weight-reducing amount of the PYY analogue or derivative thereof.

In one embodiment the invention relates to a method of reducing food intake by administration of the PYY analogue or derivative thereof. In one embodiment the invention relates to a method of inducing satiety in a subject by administration of the PYY analogue or derivative thereof. In one embodiment the invention relates to a method of reducing caloric intake in a subject by administration of the PYY analogue or derivative thereof.

In one embodiment the invention relates to a method of reducing nutrient availability by administration of the PYY analogue or derivative thereof. In one embodiment the invention relates to a method of inhibition of food intake, slowing of gastric emptying, inhibition of gastric acid secretion, and inhibition of pancreatic enzyme secretion by administration of the PYY analogue or derivative thereof. In one embodiment the invention relates to a method of treating or preventing metabolic diseases such as type 1, type 2, or gestational diabetes mellitus, obesity and other manifestations of insulin-resistance syndrome (Syndrome X) by administration of the PYY analogue or derivative thereof.

In one embodiment the invention relates to a method for altering energy metabolism in a subject by administration of the PYY analogue or derivative thereof. In one embodiment the method for altering energy metabolism in a subject comprises administration of the PYY analogue or derivative thereof. In one embodiment the invention relates a method of increasing energy expenditure and decreasing efficiency of calorie utilization in a subject by administration of the PYY analogue or derivative thereof. In one embodiment the invention relates to a method of increasing energy expenditure by administration of the PYY analogue or derivative thereof.

In one embodiment the invention relates to a method for treating and/or preventing obesity, wherein the method comprises administering a therapeutically or prophylactically effective amount of the PYY analogue or derivative thereof to a subject in need thereof. In one embodiment the subject is an obese or overweight subject. While “obesity” is generally defined as a body mass index over 30, for purposes of this disclosure, any subject, including those with a body mass index of less than 30, who needs or wishes to reduce body weight is included in the scope of “obese”. Subjects who are insulin resistant, glucose intolerant, or have any form of diabetes mellitus (e.g., type 1, 2 or gestational diabetes) can benefit from the methods disclosed herein. In one embodiment the invention relates to methods of reducing food intake, reducing nutrient availability, causing weight loss, affecting body composition, and altering body energy content or increasing energy expenditure, treating diabetes mellitus and/or improving lipid profile (including reducing LDL cholesterol and triglyceride levels and/or changing HDL cholesterol levels), wherein the method comprises administration of the PYY analogue or derivative thereof. In one embodiment the methods of the invention are used to treat or prevent conditions or disorders which can be alleviated by reducing nutrient availability in a subject comprising administration to said subject of the PYY analogue or derivative thereof, such conditions and disorders include, but are not limited to, hypertension, dyslipidemia, cardiovascular disease, eating disorders, insulin-resistance, obesity, and diabetes mellitus of any kind.

In one embodiment the invention relates to a method for treating and/or preventing obesity-related diseases, such as reduction of food intake, Syndrome X (metabolic syndrome), diabetes, type 2 diabetes mellitus or Non Insulin Dependent Diabetes Mellitus (NIDDM), hyperglycemia, insulin resistance, impaired glucose tolerance, cardiovascular disease, hypertension, atherosclerosis, coronary artery disease, myocardial infarction, peripheral vascular disease, stroke, thromboembolic diseases, hypercholesterolemia, hyperlipidemia, gallbladder disease, osteoarthritis, sleep apnea, reproductive disorders such as polycystic ovarian syndrome, or cancer of the breast, prostate, or colon by administration of the PYY analogue or derivative thereof.

In one embodiment the PYY analogue or derivative thereof provides a reduction of food intake of at least 5%, such as at least 10%, 15%, 20%, 25% or 30% compared to vehicle. In one embodiment the PYY analogue or derivative thereof provides a reduction of food intake in the range of 5-30%, such as at least 5-20%, 5-15% or 10-20% compared to vehicle. In one embodiment the PYY analogue or derivative thereof provides a reduction of body weight of at least 5%, such as at least 10%, 15%, 20%, 25% or 30% compared to vehicle. In one embodiment the PYY analogue or derivative thereof provides a reduction of body weight in the range of 5-30%, such as at least 5-20%, 5-15% or 10-20% compared to vehicle.

### Gastro-Intestinal-Related Indications

In one embodiment the invention relates to a method for treating and/or preventing a disease associated with excess intestinal electrolyte and water secretion, decreased absorption or intestinal inflammatory condition, e.g., infectious diarrhoea, inflammatory diarrhoea, short bowel syndrome, or the diarrhoea which typically occurs following surgical procedures, e.g., ileostomy. Examples of infectious diarrhoea include, without limitation, acute viral diarrhoea, acute bacterial diarrhoea (e.g., salmonella, Campylobacter, and Clostridium or due to protozoal infections), or traveller's diarrhoea (e.g., Norwalk virus or rotavirus). Examples of inflammatory diarrhoea include, without limitation, malabsorption syndrome, tropical sprue, chronic pancreatitis, ulcerative colitis, Crohn's disease, diarrhoea, and irritable bowel syndrome by administration of the PYY analogue or derivative thereof.

The PYY analogue or derivative thereof exhibits a broad range of biological activities, some related to their antisecretory and antimotility properties. The PYY analogue or derivative thereof may suppress gastrointestinal secretions by direct interaction with epithelial cells or, perhaps, by inhibiting secretion of hormones or neurotransmitters which stimulate intestinal secretion. Anti-secretory properties include inhibition of gastric and/or pancreatic secretions and can be useful in the treatment or prevention of diseases and disorders including gastritis, acute pancreatitis, Barrett's esophagus, and Gastroesophageal Reflux Disease.

The PYY analogue or derivative thereof may be useful in the treatment of any number of gastrointestinal disorders (see e.g., Harrison's Principles of Internal Medicine, McGraw-Hill Inco, N.Y., 12th Ed.) that are associated with excess intestinal electrolyte and water secretion as well as decreased absorption A method of measuring intestinal electrolyte secretion is described on page 1250 of (Eto B et al., Comparison of the antisecretory effect of endogenous forms of peptide YY on fed and fasted rat jejunum, Peptides, 1997; 18(8): 1249-55).

In one embodiment the invention relates to a method for treating and/or preventing a condition characterized by damage to the intestine (see WO 03/105763, incorporated herein by reference in its entirety) such as chemotherapy-induced diarrhoea, ulcerative colitis, inflammatory bowel disease, bowel atrophy, loss bowel mucosa, and/or loss of bowel mucosal function by administration of the PYY analogue or derivative thereof. Assays for such activity, as described in WO 03/105763, include 11 week old male HSD rats, ranging 250-300 grams housed in a 12:12 lightdark cycle, and allowed ad libitum access to a standard rodent diet (Teklad LM 485, Madison, Wis.) and water. The animals were fasted for 24 hours before the experiment. A simple and reproducible rat model of chronic colonic inflammation has been previously described by Morris G P, et al., “Hapten-induced model of chronic inflammation and ulceration in the rat colon”, Gastroenterology, 1989; 96:795-803. It exhibits a relatively long duration of inflammation and ulceration, affording an opportunity to study the pathophysiology of colonic inflammatory disease in a specifically controlled fashion, and to evaluate new treatments potentially applicable to inflammatory bowel disease in humans. Rats were anesthetized with 3% isofluorane and placed on a regulated heating pad set at 37° C. A gavage needle was inserted rectally into the colon 7 cm. The hapten trinitrobenzenesulfonic acid (TNBS) dissolved in 50% ethanol (v/v) was delivered into the lumen of the colon through the gavage needle at a dose of 30 mg/kg, in a total volume of 0.4-0.6 mL, as described in Mazelin, et al., “Protective role of vagal afferents in experimentally-induced colitis in rats”, Juton Nery Syst, 1998; 73:38 45. Control groups received saline solution (NaCl 0.9%) intracolonically. Four days after induction of colitis, the colon was resected from anesthetized rats, which were then euthanized by decapitation. Weights of excised colon and spleen were measured, and the colons photographed for scoring of gross morphologic damage. Inflammation was defined as regions of hyperemia and bowel wall thickening.

Further indications in which the PYY analogue or derivative thereof may be used as well as methods of determination of effects of said analogue or derivative in relation to said indication are described in the international application no. PCT/EP2009/055989. Specifically, the PYY analogue or derivative thereof may be useful for treatment and/or prevention of indications selected from the group consisting of potentiating, inducing, enhancing or restoring glucose responsiveness in pancreatic islets or cells, treating or preventing conditions associated with metabolic disorders, anxiety, hypotension, rhinitis, promoting wound healing, decreasing time of recreation after surgery, promoting arteriogenesis as described in the international application no. PCT/EP2009/055989, wherein methods of determination of the effect of said analogue or derivative in at least one of said indications are also described.

In one embodiment an acute test may be performed where the PYY analogue or derivative thereof is administered to ensure that said analogue or derivative have the intended effect in the subject to be treated before a chronic treatment is started. Through these means it is ensured that only subjects who are susceptible to treatment with the PYY analogue or derivative thereof are treated with said analogue or derivative.

### Syntheses

PYY analogues or derivatives thereof of the invention may be synthesized by standard solid phase peptide synthesis (SPPS), using either an automated peptide synthesizer, or traditional bench synthesis. The solid support can be, e.g., Tentagel S RAM, chlorotrityl (Cl) or Wang (OH) resin, all of which are readily available commercially. The active amino or hydroxyl groups of those resins react readily with the carboxyl group of an N-Fmoc amino acid, thereby covalently binding it to the polymer via a linkage to a linker attached to the resin. The resin-bound Fmoc-amino acid may be deprotected by exposure to a mixture of 20% piperidine in N-methylpyrrolidinone (NMP) which readily cleaves the Fmoc-group. The subsequent amino acid is coupled using a coupling reagent and followed by another deprotection of the Fmoc-group. Examples of reagents facilitating the coupling of incoming amino acids to the resin-bound amino acid chain are: diisopropylcarbodiimide (DIC), tetra-methyluronium hexafluorophosphate (HATU), O-(1H-benzotriazole-1-yl)-N,N,N\N′-tetramethyluronium hexafluorophosphate (HBTU), O-(1H-benzotriazole-1-yl)-N,N,N′,N′-tetramethyluronium tetrafluoroborate (TBTU), 1H-hydroxybenzotriazole (HOBt).

The SPPS is continued a stepwise manner until the desired sequence is obtained. At the end of the synthesis, the resin-bound protected peptide is deprotected cleaving the protection groups on the side chains and also cleaving the peptide from the resin. This is done with trifluoroacetic acid (TFA) containing scavengers such as triisopropylsilane (TIPS). The peptide is then precipitated in diethylether and isolated. Peptide synthesis by solution chemistry rather than solid phase chemistry is also feasible.

It may be desirable to purify the PYY analogues and derivatives thereof generated by the present invention. Peptide purification techniques are well known to those of skill in the art. These techniques involve, at one level, the crude fractionation of the cellular milieu to peptide and non-peptide fractions. Having separated the peptide from other proteins, the peptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography, polyacrylamide gel electrophoresis, and isoelectric focusing. A particularly efficient method of purifying peptides is reverse phase HPLC, followed by characterization of purified product by liquid chromatography/mass spectrometry (LC/MS) and Matrix-Assisted Laser Desorption Ionization (MALDI) mass spectrometry. Additional confirmation of purity is obtained by determining amino acid analysis.

Certain embodiments of the present invention concern the purification, and in particular embodiments, the substantial purification, of a peptide, including the PYY analogue or derivative thereof according to the invention. The term “purified peptide” as used herein, is intended to refer to a composition, isolatable from other components, wherein the peptide is purified to any degree relative to its naturally obtainable state. A purified peptide therefore also refers to a peptide, free from the environment in which it may naturally occur. Generally, “purified” will refer to a peptide composition that has been subjected to fractionation to remove various other components, and which composition substantially retains its expressed biological activity. Where the term “substantially purified” is used, this designation will refer to a composition in which the peptide forms the major component of the composition, such as constituting about 50%, about 60%, about 70%, about 80%, about 90%, about 95% or more of the peptides in the composition.

Various techniques suitable for use in peptide purification will be well known to those of skill in the art. These include, e.g., precipitation with ammonium sulphate, PEG, antibodies, and the like; heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the peptides always be provided in their most purified state. Indeed, it is contemplated that less substantially purified products will have utility in certain embodiments. Partial purification may be accomplished by using fewer purification steps in combination, or by utilizing different fopins of the same general purification scheme. For example, it is appreciated that a cation-exchange column chromatography performed, utilizing an HPLC apparatus, will generally result in a greater “-fold” purification than the same technique utilizing a low pressure chromatography system. Methods exhibiting a lower degree of relative purification may have advantages in total recovery of protein product, or in maintaining the activity of an expressed protein.

One may optionally purify and isolate PYY analogues and derivatives thereof of the invention from other components obtained in the process. Methods for purifying a peptide can be found in U.S. Pat. No. 5,849,883. These documents describe specific exemplary methods for the isolation and purification of G-CSF compositions that may be useful in isolating and purifying PYY analogues and derivatives thereof of the invention. Given the disclosure of these patents, it is evident that one of skill in the art would be well aware of numerous purification techniques that may be used to purify PYY analogues and derivatives thereof of the invention from a given source.

Also it is contemplated that a combination of anion exchange and immunoaffinity chromatography may be employed to produce purified compositions of PYY analogue or derivatives thereof.

### EMBODIMENTS OF THE INVENTION

### Analogue

1. A PYY analogue or a derivative thereof with improved stability against C-terminal proteolytic breakdown which breakdown decreases functionality of said analogue or derivative as compared to human PYY(1-36) or human PYY(3-36).
2. The PYY analogue or a derivative thereof according to any one of the preceding embodiments, wherein said PYY analogue does not comprise [N-methyl Tyr36] or [D-Ala3] in any one of PYY(1-36), PYY(2-36), PYY(3-36), PYY(4-36) or PYY(5-36).
3. The PYY analogue or a derivative thereof according to any one of the preceding embodiments, wherein said functionality is determined by Assay (IV) as described herein, in vitro half life in plasma.
4. The PYY analogue or a derivative thereof according to any one of the preceding embodiments, wherein said analogue or derivative comprises the amino acid sequence of the formula (I):

Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅-Xaa₂₆-Xaa₂₇-Xaa₂₈-Xaa₂₉-Xaa₃₀-Xaa₃₁-Xaa₃₂-Xaa₃₃-Xaa₃₄-Xaa₃₅-Xaa₃₆ Formula (I)

wherein
Xaa₁ is Tyr, Phe, Ala, 2,3-diaminopropionic acid, 2,4-diaminobutyric acid, ornithine, Lys or absent;
Xaa₂ is Pro, Ala, Leu, Phe, hydroxyproline, 2,3-diaminopropionic acid, 2,4-diaminobutyric acid, ornithine, Lys or absent;
Xaa₃ is Ile, Val, Leu (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₄ is Lys, Glu, 2,3-diaminopropionic acid, 2,4-diaminobutyric acid, ornithine or Lys;
Xaa₅ is Pro, 2,3-diaminopropionic acid, 2,4-diaminobutyric acid, ornithine or Lys;
Xaa₆ is Glu, 2,3-diaminopropionic acid, 2,4-diaminobutyric acid, ornithine or Lys;
Xaa₇ is Ala, 2,3-diaminopropionic acid, 2,4-diaminobutyric acid, ornithine or Lys;
Xaa₈ is Pro, Ala, 2,3-diaminopropionic acid, 2,4-diaminobutyric acid, ornithine or Lys;
Xaa₉ is Gly, 2,3-diaminopropionic acid, 2,4-diaminobutyric acid, ornithine or Lys;
Xaa₁₀ is Glu, 2,3-diaminopropionic acid, 2,4-diaminobutyric acid, ornithine or Lys;
Xaa₁₁ is Asp, Ala, 2,3-diaminopropionic acid, 2,4-diaminobutyric acid, ornithine or Lys;
Xaa₁₂ is Ala, 2,3-diaminopropionic acid, 2,4-diaminobutyric acid, ornithine or Lys;
Xaa₁₃ is Ser, 2,3-diaminopropionic acid, 2,4-diaminobutyric acid, ornithine or Lys;
Xaa₁₄ is Pro or hydroxyproline;
Xaa₁₅ is Glu, 2,3-diaminopropionic acid, 2,4-diaminobutyric acid, ornithine or Lys;
Xaa₁₆ is Glu, 2,3-diaminopropionic acid, 2,4-diaminobutyric acid, ornithine or Lys;
Xaa₁₇ is Leu, Val, Ile, homoleucine, norleucine, (1-aminocyclopentyl) carboxylic acid, (1-aminocyclohexyl) carboxylic acid or 1-aminobutyric acid;
Xaa₁₈ is Asn, 2,3-diaminopropionic acid, 2,4-diaminobutyric acid, ornithine or Lys;
Xaa₁₉ is Arg, Ala, 2,3-diaminopropionic acid, 2,4-diaminobutyric acid, ornithine or Lys;
Xaa₂₀ is Tyr, Ala, Phe, 3-pyridylalaine, 2,3-diaminopropionic acid, 2,4-diaminobutyric acid, ornithine or Lys;
Xaa₂₁ is Tyr, Ala, Phe, 3-pyridylalaine, 2,3-diaminopropionic acid, 2,4-diaminobutyric acid, ornithine or Lys;
Xaa₂₂ is Ala, 2,3-diaminopropionic acid, 2,4-diaminobutyric acid, ornithine or Lys;
Xaa₂₃ is Ser, 2,3-diaminopropionic acid, 2,4-diaminobutyric acid, ornithine or Lys;
Xaa₂₄ is Leu, Ile, Val, homoleucine, norleucine, (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₂₅ is Arg, Ala, His, Tyr, aminoisobutyric acid, 2,3-diaminopropionic acid, 2,4-diaminobutyric acid, ornithine or Lys;
Xaa₂₆ is His, Arg, 2,3-diaminopropionic acid, 2,4-diaminobutyric acid, ornithine or Lys;
Xaa₂₇ is Tyr, Ala, Phe, homoPhe or 3-pyridylalanine, 2,3-diaminopropionic acid, 2,4-diaminobutyric acid, ornithine or Lys;
Xaa₂₈ is Ile, Val, Leu, homoleucine, norleucine, (1-aminocyclopentyl) carboxylic acid, (1-aminocyclohexyl) carboxylic acid, aminoisobutyric acid, 1-aminobutyric acid, 2,3-diaminopropionic acid, 2,4-diaminobutyric acid, ornithine or Lys;
Xaa₂₉ is Asn, Gln, 2,3-diaminopropionic acid, 2,4-diaminobutyric acid, ornithine or Lys;
Xaa₃₀ is Met, Leu, Val, Ile, homoleucine, aminoisobutyric acid, norleucine, (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₃₁ is Leu, Val, Ile, aminoisobutyric acid, homoleucine, norleucine, (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₃₂ is Ser, Thr, 2,3-diaminopropionic acid, 2,4-diaminobutyric acid, ornithine or Lys;
Xaa₃₃ is Arg, N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid;
Xaa₃₄ is Gln, Asn, His, Pro, N-methyl Gln, β-homo Gln, (2-Carbamoyl-ethylamino)-acetic acid, N-methyl Asn or N-methyl His;
Xaa₃₅ is Arg, N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid, or Amino-(1-carbamimidoyl-piperidin-4-yl)-acetic acid;
Xaa₃₆ is Tyr, Phe, N-methyl Tyr, C-α-methyl Phe, 3-pyridylalanine or (4-Hydroxybenzylamino)-acetic acid.
5. The PYY analogue or derivative thereof according to any of the preceding embodiments, wherein the peptide is derived from a vertebrate such as a mammal, e.g., a human.
6. The PYY analogue or a derivative thereof according to any one of the preceding embodiments, wherein said analogue or derivative comprises at least one amino acid residue substituted into a proteinogenic or non-proteinogenic amino acid residue selected from the group consisting of

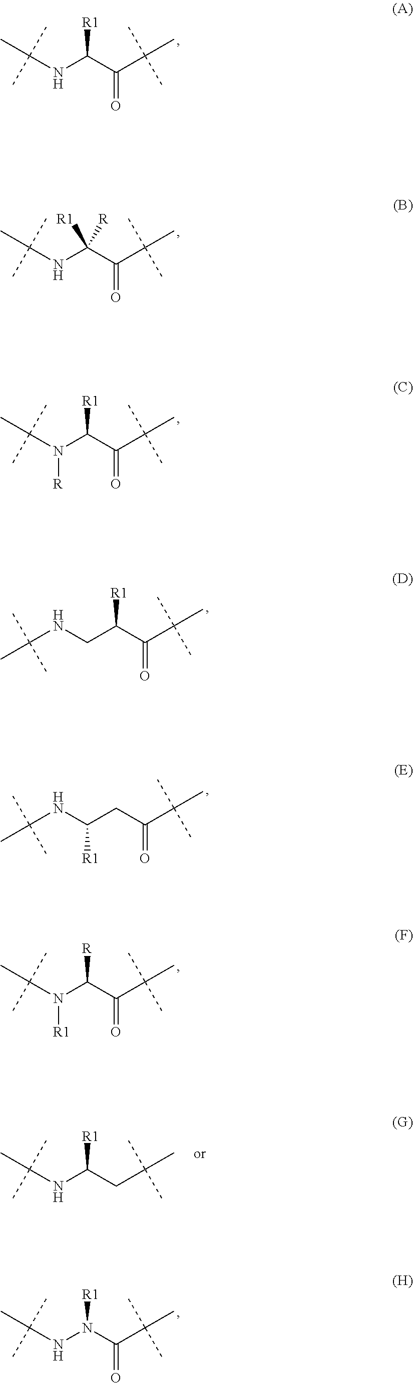

wherein R1 is side a chain of an amino acid; and R is H or C1-C12 alkyl.
7. The PYY analogue or a derivative thereof according to any one of the preceding embodiments, wherein said analogue or derivative comprises the amino acid residue represented by formula (C)

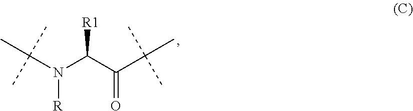

wherein
R1 is a side chain of an amino acid and
R is selected from the group consisting of alkyl, benzyl or phenyl.
8. The PYY analogue or a derivative thereof according to embodiment 7, wherein said analogue or derivative comprises the amino acid residue represented by formula (C) in position 35.
9. The PYY analogue or a derivative thereof according to any one of the preceding embodiments, wherein said analogue or derivative comprises at least one peptide bond which is altered to a reduced peptide bond or a peptide bond isoster, such as a tetrazole, a sulphonamide or an azide.
10. The PYY analogue or a derivative thereof according to any one of the preceding embodiments, wherein said analogue or derivative comprises at least one substitution with an N-methyl amino acid, N-substituted glycine, homo amino acid, beta-amino acid, beta-homo amino acid, C-alpha-methyl amino, an amino acid with shortened sidechain, an amino acid with a methylated sidechain, an alpha-methyl amino acid, a peptoid or a beta-amino acid.
11. The PYY analogue or a derivative thereof according to any one of the preceding embodiments, wherein said analogue or derivative comprises at least one substitution in positions selected from the group consisting of position 36, 35, 34, 33, 32, 31, 30, 29, 28, 27, 26, 25, 21, 20, 19 and 4.
12. The PYY analogue or a derivative thereof according to any one of the preceding embodiments, wherein Xaa₂₀ is Ala.
13. The PYY analogue or a derivative thereof according to any one of the preceding embodiments, wherein Xaa₂₅ is selected from the group consisting of His, aminoisobutyric acid (Aib) and Tyr.
14. The PYY analogue or a derivative thereof according to any one of the preceding embodiments, wherein Xaa₂₇ is Ala.
15. The PYY analogue or a derivative thereof according to any one of the preceding embodiments, wherein Xaa₃₃ is N-methyl Arg, methyllysine, dimethyllysine, trimethyllysine, 2-amino-3-guanidino-propionic acid, 2-amino-4-guanidino-butyric acid, monomethyllarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)butyric acid, 2-Amino-3-(4-guanidino-phenyl)-propionic acid or Amino-(1-carbamimidoylpiperidin-4-yl)-acetic acid.
16. The PYY analogue or a derivative thereof according to any one of the preceding embodiments, wherein Xaa₃₃ is N-methyl Arg.
17. The PYY analogue or a derivative thereof according to any one of the preceding embodiments, wherein Xaa₃₄ is Pro, N-methyl Gln, β-homo Gln, (2-Carbamoyl-ethylamino)acetic acid, N-methyl Asn or N-methyl His.
18. The PYY analogue or a derivative thereof according to any one of the preceding embodiments, wherein Xaa₃₄ is N-methyl Gln.
19. The PYY analogue or a derivative thereof according to any one of the preceding embodiments, wherein Xaa₃₄ is β-homo Gln.
20. The PYY analogue or a derivative thereof according to any one of the preceding embodiments, wherein Xaa₃₅ is N-methyl Arg, methyllysine, dimethyllysine, trimethyllysine, 2-amino-3-guanidino-propionic acid, 2-amino-4-guanidino-butyric acid, monomethyllarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)butyric acid, 2-Amino-3-(4-guanidino-phenyl)-propionic acid, or Amino-(1-carbamimidoyl-piperidin-4-yl)-acetic acid.
21. The PYY analogue or a derivative thereof according to any one of the preceding embodiments, wherein Xaa₃₅ is N-methyl Arg.
22. The PYY analogue or a derivative thereof according to any one of the preceding embodiments, wherein Xaa₃₅ is N-methyl Gln.
23. The PYY analogue or a derivative thereof according to any one of the preceding embodiments, wherein Xaa₃₆ is Phe, N-methyl Tyr, C-alpha-methyl Phe, 3-pyridylalanine or (4-Hydroxy-benzylamino)-acetic acid.
24. The PYY analogue or a derivative thereof according to any one of the preceding embodiments, wherein Xaa₃₆ is N-methyl Tyr, C-alpha-methyl Phe, 3-pyridylalanine or (4-Hydroxy-benzylamino)-acetic acid.
25. The PYY analogue or a derivative thereof according to any one of the preceding embodiments, wherein Xaa₃₆ is α-methyl Phe.
26. The PYY analogue or a derivative thereof according to any one of the preceding embodiments, wherein at least one of Xaa₃₃, Xaa₃₄, Xaa₃₅ and Xaa₃₆ is selected from the group consisting of
Xaa₃₃ is N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid;
Xaa₃₄ is N-methyl Gln, β-homo Gln, (2-Carbamoyl-ethylamino)-acetic acid, N-methyl Asn or N-methyl His;
Xaa₃₅ is N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid, or Amino-(1-carbamimidoyl-piperidin-4-yl)-acetic acid;
Xaa₃₆ is N-methyl Tyr, C-α-methyl Phe, 3-pyridylalanine or (4-Hydroxy-benzylamino)acetic acid.
27. The PYY analogue or a derivative thereof according to any one of the preceding embodiments, wherein Xaa₁ and Xaa₂ are absent or Xaa₁, Xaa₂, Xaa₃ and Xaa₄ are absent.
28. The PYY analogue or a derivative thereof according to any one of the preceding embodiments, wherein said analogue or derivative is selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 23, SEQ ID NO: 24, SEQ ID NO: 27 and SEQ ID NO: 28.
29. The PYY analogue or a derivative thereof according to any one of the preceding embodiments, wherein said analogue or derivative is SEQ ID NO: 32 or SEQ ID NO: 34.
30. The PYY analogue or a derivative thereof according to any one of the preceding embodiments, wherein said analogue or derivative is selected from the group consisting of SEQ ID NO: 11, SEQ ID NO: 13, SEQ ID NO: 16, SEQ ID NO: 17, SEQ ID NO: 18, SEQ ID NO: 19, SEQ ID NO: 23, SEQ ID NO: 24, SEQ ID NO: 28, SEQ ID NO: 32 and SEQ ID NO: 34.
31. The PYY analogue or a derivative thereof according to any one of the preceding embodiments, wherein said analogue or a derivative does not comprise a derivatisation group and wherein said analogue or a derivative is not
[N-methyl Arg33] PYY(3-36),
[N-methyl Gln34] PYY(3-36),
[N-methyl Arg35] PYY(3-36),
[N-methyl Tyr36] PYY(1-36), or
[N-methyl Tyr36] PYY(3-36).

### Derivative

32. The PYY analogue or a derivative thereof according to any one of the preceding embodiments, wherein said analogue or derivative comprises at least one derivatisation with a serum albumin binding side chain comprising a distal carboxylic acid or a distal tetrazole group.
33. The PYY analogue or a derivative thereof according to according to any one of the preceding embodiments, wherein said proteinogenic or non-proteinogenic amino acid residue is in at least one position selected from the group consisting of position 33, 34, and 36.
34. The PYY analogue or a derivative thereof according to any one of the preceding embodiments, wherein said analogue or derivative comprises at least one derivatisation with either A-B-C-D- or A-C-D- or A-B-C- or A-C-,
wherein A- is

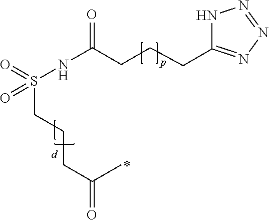

wherein p is selected from the group consisting of 10, 11, 12, 13 and 14, and d is selected from the group consisting of 0, 1, 2, 3, 4 and 5, and
-B- is selected from the group consisting of

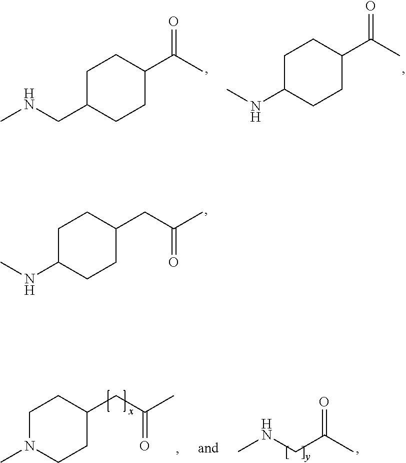

wherein x is selected from the group consisting of 0, 1, 2, 3 and 4, and y is selected from the group consisting of 1, 2, 3, 4, 5, 6, 7, 8, 9, 10, 11 and 12,

### or A- is

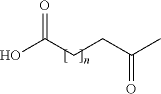

wherein n is selected from the group consisting of 12, 13, 14, 15, 16 17, 18 and 19, and B is selected from the group consisting of

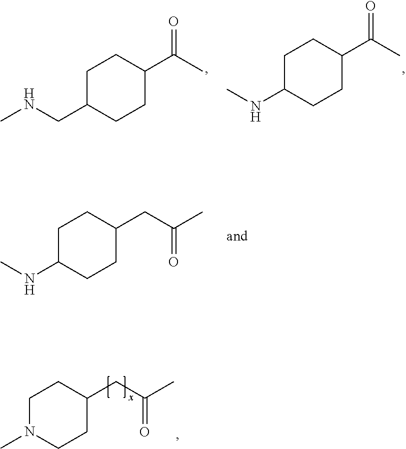

wherein x is selected from the group consisting of 0, 1, 2, 3 and 4, and
-C- is selected from the group consisting of

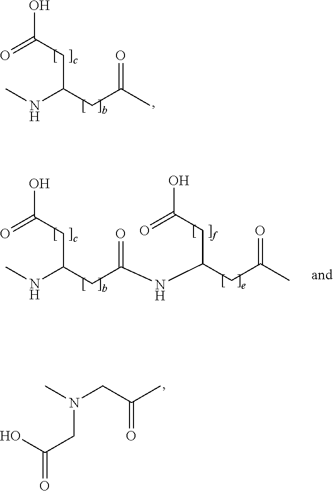

wherein b and e are each independently selected from the group consisting of 0, 1 and 2, and c and f are each independently selected from the group consisting of 0, 1 and 2 with the proviso that b is 1 or 2 when c is 0, or b is 0 when c is 1 or 2, and e is 1 or 2 when f is 0, or e is 0 when f is 1 or 2, and
-D- is attached to said amino acid residue and is a spacer.
   35. The PYY analogue or a derivative thereof according to embodiment 34, wherein said derivatisation with either A-B-C-D- or A-C-D- or A-B-C- or A-C- is attached to an amino acid residue, such as lysine, and/or the N-terminal amino group and/or the C-terminal amino group.
   36. The PYY analogue or derivative thereof according to any of embodiments 33-35, wherein the serum albumin binding side chain is attached to an amino group of the side chain of an amino acid of the peptide backbone.
   37. The PYY analogue or derivative thereof according to any of embodiments 33-36, wherein the serum albumin binding side chain is attached to an amino group of the side chain of an amino acid of the peptide backbone selected from the group consisting of 2,3-diaminopropionic acid, 2,4-diaminobutyric acid, ornithine, and Lys.
   38. The PYY analogue or derivative thereof according to any of embodiments 33-37, wherein the spacer, -D-, comprises at least one 8-amino-3,6-dioxaoctanoic acid (Oeg) molecule.
   39. The PYY analogue or derivative thereof according to any of embodiments 33-38, wherein the serum albumin binding side chain comprises at least 14 carbon atoms, such as 16, 18 or 20 carbon atoms.
   40. The PYY analogue or derivative thereof according to any of embodiments 33-39, wherein the serum albumin binding side chain comprises an amide.
   41. The PYY analogue or derivative thereof according to any of embodiments 33-40, wherein the serum albumin binding side chain comprises a distal carboxylic acid group or a distal tetrazole group.
   42. The PYY analogue or derivative thereof according to any of embodiments 33-41, wherein the serum albumin binding side chain comprises an alkyl chain with at least 14 carbon atoms comprising a distal carboxylic acid or a distal tetrazole group.
   43. The PYY analogue or derivative thereof according to any of embodiments 33-42, wherein the serum albumin binding side chain comprises a C18 dicarboxylic acid or a C16 dicarboxylic acid.
   44. The PYY analogue or derivative thereof according to any of embodiments 33-43, wherein the serum albumin binding side chain comprises a C16 carboxylic acid.
   45. The PYY analogue or a derivative thereof according to any one of embodiments 33-44, wherein said derivatisation is 2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl or 2-(2-{2-[2-(2-{2-[4-Carboxy-4-(17-carboxyheptadecanoylamino)butyrylamino]ethoxy}-ethoxy)acetylamino]-ethoxy}ethoxy)acetyl.
   46. The PYY analogue or a derivative thereof according to any one of embodiments 33-45, wherein said analogue or derivative is selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 20, SEQ ID NO: 21, SEQ ID NO: 22, SEQ ID NO: 25, SEQ ID NO: 26, SEQ ID NO: 29, SEQ ID NO: 30 and SEQ ID NO: 31.
   47. The PYY analogue or a derivative thereof according to any one of embodiments 33-46, wherein said analogue or derivative is SEQ ID NO: 33 or SEQ ID NO: 35.

### Determination of Functionality

48. The PYY analogue or a derivative thereof according to any of the preceding embodiments, wherein said functionality is determined by an assay selected from the group consisting of Assay (IV), Assay (I), Assay (V), Assay (VII), Assay (VI), Assay (VIII) and Assay (IX) as described herein.
49. The PYY analogue or a derivative thereof according to any of the preceding embodiments, wherein said functionality is determined by an assay selected from the group consisting of Assay (II) and Assay (III) as described herein.

### Functional Embodiments

50. The PYY analogue or a derivative thereof according to any of the preceding embodiments, wherein said analogue or derivative has protracted properties compared to human PYY(3-36).
51. The PYY analogue or a derivative thereof according to any of the preceding embodiments, wherein the half life is at least 2 times, such as at least 3, 4, 5 or 8 times, the half life of PYY(3-36).
52. The PYY analogue or a derivative thereof according to any of the preceding embodiments, wherein the half life is at least 50 times, such as at least 75, 100, 200 or 300 times, the half life of PYY(3-36).
53. The PYY analogue or a derivative thereof according to embodiment 36 or 37, wherein the half life is determined by Assay (IV) or Assay (V) described herein.
54. The PYY analogue or derivative thereof according to any of the preceding embodiments, wherein said analogue or derivative is suitable for administration in a dosing regime selected from group consisting of a once-daily, a once-weekly, a twice-monthly and a once-monthly dosing regime.
55. The PYY analogue or derivative thereof according to any of the preceding embodiments, wherein said analogue or derivative shows improved PK profile compared to human PYY, PYY(3-36) or PYY(3-36) with a serum albumin binding group identical to that of said analogue or derivative.
56. The PYY analogue or derivative thereof according to any of the preceding embodiments, wherein said analogue or derivative shows protracted properties compared to human PYY, PYY(3-36) or PYY(3-36) with a serum albumin binding group identical to that of said analogue or derivative.
57. The PYY analogue or derivative thereof according to any of the preceding embodiments, wherein said analogue or derivative shows improved half life in vivo compared to human PYY, PYY(3-36) or PYY(3-36) with a serum albumin binding group identical to that of said analogue or derivative.
58. The PYY analogue or derivative thereof according to any of the preceding embodiments, wherein a therapeutically effective dose of said analogue or derivative causes less side effects compared to human PYY, PYY(3-36) or PYY(3-36) with a serum albumin binding group identical to that of said analogue or derivative.
59. The PYY analogue or a derivative thereof according to any one of the preceding embodiments, wherein said analogue or derivative has increased Y1/Y2 receptor potency ratio compared to hPYY(3-36).
60. The PYY analogue or a derivative thereof according to any one of the preceding embodiments, wherein said analogue or derivative has increased Y5/Y2 receptor potency ratio compared to hPYY(3-36).

### Compositions

61. A composition comprising the PYY analogue or a derivative thereof as defined in any of the preceding embodiments and at least one pharmaceutical excipient.

### Indications

62. The PYY analogue or a derivative thereof according to any of the preceding embodiments for use in the treatment of a condition responsive to Y receptor modulation.
63. The PYY analogue or a derivative thereof according to embodiment 62, wherein the condition responsive to Y receptor modulation is obesity.
64. The PYY analogue or a derivative thereof according to embodiment 62 or 63, wherein said analogue or derivative is administered once-daily, twice-weekly or once-weekly.
65. Use of the PYY analogue or derivative thereof as defined in any of embodiments 1-60 for the preparation of a medicament for the treatment of a condition responsive to Y receptor modulation, such as obesity or obesity-related diseases, e.g., reduction of food intake.
66. Use of the PYY analogue or derivative thereof as defined in any of embodiments 1-60 for administration in a mammal, wherein said analogue or derivative shows protracted properties compared to human PYY, PYY(3-36) or PYY(3-36) with a serum albumin binding group identical to that of said analogue or derivative.
67. A method of treatment of a condition responsive to Y receptor modulation by administration of the PYY analogue or a derivative thereof as defined in any of embodiments 1-60.
68. A method according to embodiment 67, wherein said condition responsive to Y receptor modulation is obesity.
69. A method according to embodiment 67 or 68, wherein said analogue or derivative is administered once-daily, twice-weekly or once-weekly.
70. A method of treatment according to any one of embodiments 67-69, wherein the condition responsive to Y receptor modulation is obesity-related diseases, such as reduction of food intake, Syndrome X (metabolic syndrome), diabetes, type 2 diabetes mellitus or Non Insulin Dependent Diabetes Mellitus (NIDDM), hyperglycemia, insulin resistance, or impaired glucose tolerance.
71. A method of treatment according to any one of embodiments 67-70, wherein the condition responsive to Y receptor modulation is obesity-related diseases, such as polycystic ovary syndrome (PCOS).
72. A method of treatment according to any one of embodiments 67-71, wherein the condition responsive to Y receptor modulation is an obesity-related cardiovascular disease such as hypertension, atherosclerosis, coronary artery disease, myocardial infarction, peripheral vascular disease, stroke, thromboembolic diseases, hypercholesterolemia, or hyperlipidemia.
73. A method of treatment according to any one of embodiments 67-72, wherein the condition responsive to Y receptor modulation is diarrhoea such as infectious diarrhoea, inflammatory diarrhoea, chemotherapy-induced diarrhoea, short bowel syndrome, or the diarrhoea which typically occurs following surgical procedures, e.g., ileostomy.
74. A method of treatment according to any one of embodiments 67-73, wherein the condition responsive to Y receptor modulation is a condition characterized by damage to the intestine such as chemotherapy-induced diarrhoea, ulcerative colitis, Crohns disease, bowel atrophy, loss of bowel mucosa, and/or loss of bowel mucosal function.
75. A method of treatment according to any one of embodiments 67-74, wherein the condition responsive to Y receptor modulation is an intestinal inflammatory condition such as ulcerative colitis or Crohn's disease.
76. A method of treatment according to any one of embodiments 67-75, wherein the condition responsive to Y receptor modulation is allergic or non-allergic rhinitis.
77. A method of treatment according to any one of embodiments 67-76, wherein the condition responsive to Y receptor modulation is anxiety.
78. A method of treatment according to any one of embodiments 67-77, wherein said analogue or derivative shows improved PK profile compared to human PYY, PYY(3-36) or PYY(3-36) with a serum albumin binding group identical to that of said analogue or derivative.
79. A method of treatment according to any one of embodiments 67-78, wherein said analogue or derivative shows protracted properties compared to human PYY, PYY(3-36) or PYY(3-36) with a serum albumin binding group identical to that of said analogue or derivative.
80. A method of treatment according to any one of embodiments 67-79, wherein said analogue or derivative shows improved half life in vivo compared to human PYY, PYY(3-36) or PYY(3-36) with a serum albumin binding group identical to that of said analogue or derivative.
81. A method of treatment according to any one of embodiments 67-80, wherein a therapeutically effective dose of said analogue or derivative causes less side effects compared to human PYY, PYY(3-36) or PYY(3-36) with a serum albumin binding group identical to that of said analogue or derivative.

### EXAMPLES

Abbreviations used:

| Abbreviation | Meaning |
|---|---|
| r.t: | Room temperature |
| DIPEA: | Diisopropylethylamine |
| H2O: | Water |
| CH3CN: | Acetonitrile |
| DMF: | N,N-dimethylformamide |
| HBTU: | 2-(1H-Benzotriazol-1-y1-)-1,1,3,3 |
| | tetramethyluronium |
| | hexafluorophosphate |
| Fmoc: | 9 H-fluoren-9-ylmethoxycarbonyl |
| Boc: | tert butyloxycarbonyl |
| OtBu: | tert butyl ester |
| tBu: | tert butyl |
| Trt: | Triphenylmethyl |
| Pmc: | 2,2,5,7,8-Pentamethyl-chroman- |
| | 6-sulfonyl |
| Dde: | 1-(4,4-Dimethyl-2,6- |
| | dioxocyclohexylidene)ethyl |
| ivDde: | 1-(4,4-Dimethyl-2,6- |
| | dioxocyclohexylidene)- |
| | 3-methylbutyl |
| Mtt: | 4-methyltrityl |
| Mmt: | 4-methoxytrityl |
| DCM: | Dichloromethane |
| TIPS: | triisopropylsilane) |
| TFA: | trifluoroacetic acid |
| Et2O: | Diethylether |
| NMP: | 1-Methyl-pyrrolidin-2-one |
| DIPEA: | Diisopropylethylamine |
| HOAt: | 1-Hydroxy-7-azabenzotriazole |
| HOBt: | 1-Hydroxybenzotriazole |
| DIC: | Diisopropylcarbodiimide |
| MW: | Molecular weight |

### Example 1

### Synthesis of Resin Bound Peptide, SPPS Method

The protected peptidyl resin was synthesized according to the Fmoc strategy on a Prelude Solid Phase Peptide Synthesizer from Protein Technologies in 0.25 mmol scale using DIC and HOAt mediated couplings in NMP. The starting resin used for the synthesis of the peptide amides was Rink-Amide resin. The protected amino acid derivatives used were standard Fmoc-amino acids (supplied from e.g. Anaspec, Bachem, Iris Biotech, Watanabe, or Novabiochem). In some cases the Fmoc-amino acids were custom synthesized using methods known in the art. The epsilon amino group of lysines to be acylated were protected with Mtt. The synthesis of the peptides may in some cases be improved by the use of dipeptides, e.g., pseudoprolines from Novabiochem, Fmoc-Ser(tbu)-ψSer(Me,Me)—OH, see e.g. catalogue from Novobiochem 2002/2003 or newer version, or W.R. Sampson (1999), J. Pep. Sci. 5, 403.

Procedure for Cleaving the Peptide off the Resin

After synthesis the resin was washed with DCM and dried, and the peptide was cleaved from the resin by a 2 hour treatment with TFA/TIPS/water (92.5/5/2.5) or TFA/TIPS (95/5) followed by precipitation with diethylether. The peptide was redissolved in 30% acetic acid or similar solvent and purified by standard RP-HPLC on a C18 column using acetonitrile/TFA. The identity of the peptide was confirmed by MALDI-MS.

Procedure for Removal of Mtt-Protection

The resin was placed in a syringe and treated with hexafluoroisopropanol for 2×10 min to remove the Mtt group. The resin was then washed with DCM and NMP as described above.

Procedure for Attachment of Sidechains to Lysine Residue

The albumin binding side chain A-B-C-D, A-C-D, A-B-C or A-B can be attached to the peptide either by acylation to resin bound peptide or acylation in solution to the unprotected peptide using standard acylating reagent such as but not limited to DIC, HOBt/DIC, HOAt/DIC, or HBTU.

Procedure for Removal of Fmoc-Protection

The resin (0.25 mmol) was placed in a filter flask in a manual shaking apparatus and treated with N-methylpyrrolidone/methylene chloride (1:1) (2×20 ml) and with N-methylpyrrolidone (1×20 ml), a solution of 20% piperidine in N-methylpyrrolidone (3×20 ml, 10 min each). The resin was washed with N-methylpyrrolidone (2×20 ml), N-methylpyrrolidone/Methylene chloride (1:1) (2×20 ml) and methylene chloride (2×20 ml).

### Procedures for Analysis of Product

MALDI-MS

Molecular weights of the peptides were determined using matrix-assisted laser desorption time of flight mass spectroscopy (MALDI-MS), recorded on a Microflex (Bruker). A matrix of alpha-cyano-4-hydroxy cinnamic acid was used. The molecular weight of the product was calculated based on the result of MALDI-MS analysis.

UPLC (Method 04 A3 1)

The UPLC-analysis was performed using a Waters Acquity UPLC system fitted with a Waters Acquity UPLC HSS T3 1.8 μm, 2.1×150 mm column. UV detections were collected at 214 and 254 nm. Oven temperature was 30 C. The following eluents were used; eluent A) 0.25M ammoniumbicarboate in water/acetonitrile (90:10) and eluent B) acetonitrile/water (70:30). The column was equilibrated with the following eluent composition: 75% eluent A, 25% eluent B. After injection, the sample was eluted at 0.4 ml/min with gradient of 25% to 55% eluent B in eluent A during 16 min.

UPLC (Method 04 A4 1)

The UPLC-analysis was performed using a Waters Acquity UPLC system fitted with a Waters Acquity UPLC HSS T3 1.8 μm, 2.1×150 mm column. UV detections were collected at 214 and 254 nm. Oven temperature was 30 C. The following eluents were used; eluent A) 0.25M ammoniumbicarboate in water/acetonitrile (90:10) and eluent B) acetonitrile/water (70:30). The column was equilibrated with the following eluent composition: 65% eluent A, 35% eluent B. After injection, the sample was eluted at 0.4 ml/min with a gradient of 35% to 65% eluent B in eluent A during 16 min.

UPLC (Method 04 A6 1)

The RP-analysis was performed using a Waters UPLC system fitted with a dual band detector. UV detections at 214 nm and 254 nm were collected using an ACQUITY UPLC BEH130, C18, 130 Å, 1.7 μm, 2.1 mm×150 mm column, 40° C. The UPLC system was connected to two eluent reservoirs containing: A: 10 mM TRIS, 15 mM ammonium sulphate, 80% H₂O, 20%, pH 7.3; B: 80% CH₃CN, 20% H₂O. The following linear gradient was used: 95% A, 5% B to 10% A, 90% B during 16 minutes at 0.35 ml/min.

UPLC (Method 07 84 1)

The UPLC-analysis was performed using a Waters Acquity UPLC system fitted with a Waters Acquity UPLC BEH 1.7 μm, 2.1×150 mm column. UV detections were collected at 214 and 254 nm. Oven temperature was 40 C. The following eluents were used; eluent A) 0.05% trifluoroacetic acid in water and eluent B) 0.0% trifluoroacetic acid in acetonitrile. The column was equilibrated with the following eluent composition: 95% eluent A, 5% eluent B. After injection, the sample was eluted at 0.4 ml/min with a gradient of 5% to 95% eluent B in eluent A during 16 min.

UPLC (Method 07 84 2)

The UPLC-analysis was performed using a Waters Acquity UPLC system fitted with a Waters Acquity UPLC BEH 1.7 μm, 2.1×50 mm column. UV detections were collected at 214 and 254 nm. Oven temperature was 40 C. The following eluents were used; eluent A) 0.05% trifluoroacetic acid in water and eluent B) 0.0% trifluoroacetic acid in acetonitrile. The column was equilibrated with the following eluent composition: 95% eluent A, 5% eluent B. After injection, the sample was eluted at 0.4 ml/min with a gradient of 5% to 95% eluent B in eluent A during 16 min.

UPLC (Method 07 84 3)

The UPLC-analysis was performed using a Waters Acquity UPLC system fitted with a Waters Acquity UPLC BEH 1.7 μm, 2.1×150 mm column. UV detections were collected at 214 and 254 nm. Oven temperature was 40 C. The following eluents were used; eluent A) 0.05% trifluoroacetic acid in water and eluent B) 0.0% trifluoroacetic acid in acetonitrile. The column was equilibrated with the following eluent composition: 95% eluent A, 5% eluent B. After injection, the sample was eluted at 0.4 ml/min with a gradient of 5% to 95% eluent B in eluent A during 16 min

UPLC (Method 07 82 1)

The UPLC-analysis was performed using a Waters Acquity UPLC system fitted with a Waters Acquity UPLC BEH 1.7 μm, 2.1×150 mm column. UV detections were collected at 214 and 254 nm. Oven temperature was 40 C. The following eluents were used; eluent A) 0.05% trifluoroacetic acid in water and eluent B) 0.0% trifluoroacetic acid in acetonitrile. The column was equilibrated with the following eluent composition: 95% eluent A, 5% eluent B. After injection, the sample was eluted at 0.4 ml/min with a gradient of 5% to 60% eluent B in eluent A during 16 min.

UPLC (Method 07 82 2)

The UPLC-analysis was performed using a Waters Acquity UPLC system fitted with a Waters Acquity UPLC BEH 1.7 μm, 2.1×50 mm column. UV detections were collected at 214 and 254 nm. Oven temperature was 40° C. The following eluents were used; eluent A) 0.05% trifluoroacetic acid in water and eluent B) 0.0% trifluoroacetic acid in acetonitrile. The column was equilibrated with the following eluent composition: 95% eluent A, 5% eluent B. After injection, the sample was eluted at 0.4 ml/min with a gradient of 5% to 60% eluent B in eluent A during 16 min.

The retention time (RT) was calculated based on the result of the UPLC analysis.

The analysis results of the compounds are shown below:

| SEQ | UPLC | | | | calcu- |
|---|---|---|---|---|---|
| ID | | RT | UPLC | MALDI- | lated |

| | | | | | | |
|---|---|---|---|---|---|---|
| NO | Method | (min) | Method | RT (min) | MS (m/z) | MW |

| | | | | | | |
|---|---|---|---|---|---|---|
| 1 | 07_B4_1 | 6.9 | 04_A3_1 | 5.9 | 4049 | 4050 |
| 2 | 07_B4_1 | 9.1 | 04_A4_1 | 8.1 | 4932 | 4933 |
| 3 | 07_B2_2 | 11.6 | 04_A4_1 | 9.0 | 4945 | 4947 |
| 4 | 07_B4_1 | 9.5 | 04_A4_1 | 14.5 | 4945 | 4947 |
| 5 | 07_B4_1 | 10.3 | 04_A4_1 | 7.7 | 4925 | 4928 |
| 6 | 07_B4_1 | 10.4 | 04_A4_1 | 8.9 | 4973 | 4876 |
| 7 | 07_B4_1 | 12.6 | 04_A3_1 | 13.0 | 4954 | 4954 |
| 8 | 07_B4_1 | 8.0 | 04_A3_1 | 10.9 | 4673 | 4673 |
| 9 | 07_B4_1 | 8.1 | 04_A3_1 | 12.3 | 4777 | 4779 |
| 10 | 07_B4_1 | 7.9 | 04_A3_1 | 10.3 | 4673 | 4673 |
| 11 | 07_B4_1 | 6.0 | 04_A3_1 | 6.9 | 4045 | 4048 |
| 12 | 07_B2_2 | 7.5 | 04_A3_1 | 8.5 | 4062 | 4064 |
| 13 | 07_B2_1 | 8.5 | 04_A3_1 | 8.5 | 4061 | 4064 |
| 14 | 07_B2_2 | 7.8 | 04_A3_1 | 8.8 | 4062 | 4064 |
| 15 | 07_B2_2 | 7.5 | 04_A3_1 | 7.8 | 4063 | 4064 |
| 16 | 07_B4_3 | 6.2 | 04_A3_1 | 9.8 | 4020 | 4021 |
| 17 | 07_B4_3 | 6.2 | 04_A3_1 | 9.8 | 4035 | 4036 |
| 18 | 07_B4_3 | 7.5 | 04_A3_1 | 7.8 | 4061 | 4064 |
| 19 | 07_B4_3 | 6.0 | 04_A3_1 | 4.7 | 4063 | 4064 |
| 20 | 07_B4_1 | 8.3 | 04_A4_1 | 5.6 | 4736 | 4737 |
| 21 | 07_B4_1 | 8.3 | 04_A4_1 | 5.4 | 4750 | 4751 |
| 22 | 07_B4_1 | 9.3 | 04_A3_1 | 9.5 | 4779 | 4779 |
| 23 | 07_B4_1 | 5.7 | 04_A6_1 | 4.4 | 4049 | 4050 |
| 24 | 07_B4_1 | 5.7 | 04_A6_1 | 4.2 | 4049 | 4050 |
| 25 | 07_B4_3 | 9.5 | 04_A6_1 | 5.8 | 4766 | 4765 |
| 26 | 07_B4_1 | 9.4 | 04_A3_1 | 6.2 | 4766 | 4765 |
| 27 | 07_B4_1 | 7.6 | 04_A3_1 | 6.2 | 4062 | 4064 |
| 28 | 07_B4_3 | 6.2 | 04_A3_1 | 9.9 | 4062 | 4064 |
| 29 | 07_B4_1 | 9.3 | 04_A3_1 | 9.5 | 4779 | 4779 |
| 30 | 07_B4_3 | 8.3 | 04_A4_1 | 4.2 | 4781 | 4779 |
| 31 | 07_B4_3 | 4.4 | 04_A4_1 | 6.5 | n/a | 4105.6 |
| 32 | 07_B4_3 | 6.5 | 04_A6_1 | 4.4 | n/a | 4106 |
| 33 | 07_B4_3 | 9.5 | 04_A6_1 | 6.4 | 4764 | 4765 |
| 34 | 07_B4_1 | 5.7 | 04_A6_1 | 4.3 | 4049 | 4050 |
| 35 | 07_B4_1 | 7.7 | 04_A6_1 | 5.8 | 4847 | 4849 |

n/a: not analysed

### Biological Assays

The utility of the PYY analogues or derivatives thereof of the present invention as pharmaceutically active agents in the reduction of weight gain and treatment of obesity in mammals (such as humans), may be demonstrated by the activity of the agonists in conventional assays and in the in vitro and in vivo assays described below.

Such assays also provide a means whereby the activities of the PYY analogues or derivatives thereof of this invention can be compared with the activities of known compounds, such as human PYY(1-36), human PYY(3-36) or human PYY(3-36) with a serum albumin binding side chain identical to that of the said derivative.

### Assay (I): Y2 Receptor ACTOne Potency Assay

This assay provides a method for determination of in vitro effect of peptides on the Y2 receptor activity using the ACTOne based FLIPR assay. ACTOne™ is an easily scaleable cAMP biosensor HTS platform for measurement of Gs and Gi coupled 7™ receptor signalling from BD Biosciences (San Jose, Calif.). The cells express a biosensor developed around a modified rat olfactory cyclic nucleotide gated (CNG) calcium channel—a fairly non-discriminatory ion channel that responds to cAMP and cGMP. The CNG has been engineered to be cAMP selective and thus function as a cAMP responsive biosensor that signals through calcium or membrane potential responsive dyes. Y2 receptor expressing ACTOne HEK-293 cells are obtained from BD Biosciences. The cells are loaded with a calcium responsive dye that only distributes in the cytoplasm. Probenecid, an inhibitor of the organic anion transporter is added to prevent the dye from leaving the cell. A phosphodiesterase inhibitor is added to prevent formatted cAMP from being degraded. Isoproterenol (a β1/β2 agonist) is added to activate the adenylate cyclase. When an Y2 or Y4 receptor agonist is added, the adenylate cyclase is inactivated. The decreased calcium concentration in the cytoplasm is then detected as a decrease in fluorescence. Together with the test substance, isoproterenol at a concentration matching EC₈₀, is added to all wells. The assay is carried out as follows: The cells are plated out in Greiner 384-well plates. 25 μl cell suspension containing 560 cells per μl are added to all wells using the Multidrop™ (384-Multidrop from Labsystems, Finland). The cell plates are then incubated in the incubator over night at 37° C. with 5% CO₂ in stacks of up to 9 plates. The cell plates are loaded with 25 μl probe from the FLIPR calcium4 kit (Molecular Devices, CA, USA) using the Multidrop™. The cell plates are returned to the incubator and incubated for 60 min. at 37° C. in stacks of up to 9 plates. The cell plates are then left at room temperature for 60 min., before use, without stacking the plates. The plates are covered with tinfoil to avoid light (the dye can be excited by the daylight, which results in higher baseline and variation). The FLIPR (FLIPRtetra from Molecular Devices, CA, USA) adds 1 μl sample and 1 μl isoproterenol (0.05 μM final concentration) at the same time. The fluorescence signal from the wells is measured 330 seconds after sample addition on the FLIPR. The EC50 is calculated as the concentration of the Y2 receptor agonist inducing 50% decrease in fluorescence signal. A reported value of 1000 nM is intended to mean at least 1000 nM as this is the detection limit of the assay.

### Assay (II)—Y1 Receptor ACTOne Potency Assay

This assay provides a method for determination of in vitro effect of peptides on the Y1 receptor activity using the ACTOne based FLIPR assay. The assay was carried out as described for Y2 receptor ACTOne potency assay (Assay (I)) except that ACTOne HEK-293 cells expressing the Y1 receptor were used. A reported value of 1000 nM is intended to mean at least 1000 nM as this is the detection limit of the assay.

### Assay (III)—Y5 Receptor IPOne Potency Assay

The IPOne-Tb assay (Cisbio, Bagnols-sur-Cèze Cedex, France) is a homogeneous time resolved fluorescence (HTRF) assay which functions as a competitive immunoassay that measures IP1 levels using cryptate labelled anti-IP1 monoclonal antibody and d2 labelled IP1, wherein IP1 is accumulated following activation of seven transmembrane receptors that couples to the Gq pathway. In the hY5 IPOne assay a HEK293 cell line stably expressing both the human Y5 receptor and the chimeric G-protein Gqi5 was used where Gqi5 ensures Gq signalling of the Gi coupled Y5 receptor. The buffers and reagents for the assay were supplied with the IPOne-Tb kit (Cisbio, Bagnols-sur-Cèze Cedex, France). The assay was carried out as follows: on the day before the assay cells were seeded at a density of 40,000 cells/well in 20 μl in 384-well small volume white tissue culture plates, Greiner #784080, and incubated overnight at 37° C. with 5% CO₂. On the day of the assay the media was removed and 10 μl stimulation buffer supplemented with 0.005% Tween-20 was added together with 5 μl agonist serial dilution. The plates were then incubated for 1 hour at 37° C. IP1-d2 and IP1-cryptate is reconstituted in lysis buffer according to the IPOne-Tb kit protocol. 3 μl of each of the IP1-d2 and IP1-cryptate working solutions was added to each well. The plate was incubated for 1 hour at room temperature. The plate was read on a Mithras LB 940 HTRF compatible reader (Berthold Technologies, Bad Wildbad, Germany) with 665 nm and 620 nm emission filters and the signal was calculated as the fluorescence ratio 665 nm/620 nm. A reported value of 1000 nM is intended to mean at least 1000 nM as this is the detection limit of the assay.

### Assay (IV): In Vitro Half Life in Plasma

1 μM of test compound was incubated at 37° C. with heparin stabilized plasma from minipig to which 20% of phosphate buffer (0.2 M, pH 7.4) was added. Aliqouts were taken at predefined time points and plasma samples were precipitated with organic solvent and the resultant supernatant was analysed by LC-MS for quantification of non-degraded test compound. The in vitro half life was determined from time profiles that were fitted as first-order kinetics.

Assay (V): PK i.v. Minipig

An assay useful for measuring the pharmacokinetic (PK) profile of the PP analogue or derivative thereof is the following mini-pig PK assay.

Five male Göttingen mini-pigs weighing approximately 18 to 30 kg from Ellegaard Göttingen Minipigs A/S, Denmark are included in the study. The mini-pigs have two central venous catheters inserted which are used for intra venous (i.v.) dosing and blood sampling. The test compound is dissolved in 50 mM K2HPO4, 0.05% tween 80, pH=8.0 to a concentration of 100-180 nmol/ml or 50 mM NaHPO4, 145 mM NaCl, 0.05% Tween 80, pH 7.4.

For comparison a control compound, such as human PP(1-36), may be administered. The pigs are dosed with 6-10 nmol test compound/kg body weight. Blood samples are taken at the following time points: pre-dose, 30 minutes, 1, 2, 4, 8, 24, 48, 72, 96, 120, 168 and 240 hours post dosing. The blood samples were collected into test tubes containing EDTA buffer for stabilization and kept on ice for max. 20 minutes before centrifugation. The centrifugation procedure to separate plasma may be: 4° C., approx. 2500 g for 10 minutes. Plasma is collected and immediately transferred to Micronic tubes stored at −20° C. until assayed.

Quantitative Assay for Plasma Samples

The test substances were assayed in plasma by Turbulent Flow Chromatography coupled to Liquid Chromatography with subsequent Mass Spectrometric Detection (TFC/LC/MS). The selectivity of the method allows various compounds to be quantitated in one sample, e.g. cassette dosing of four compounds per animal.

The concentrations of the test substance in unknown samples were calculated using the peak area as a function of amount. Calibration graphs based on plasma samples spiked with the analyte were constructed by regression analysis. Typical dynamic range for standard assay was 1-2,000 nmol/l. The method performance was assured by co-assaying quality control (QC) samples in duplicate at three concentration levels.

Stock and working solutions of analytes were prepared in plasma and incubated by 37° C. for 1 hour.

Sample Preparation: 40.0 μl EDTA-plasma was added 160 μl 50% methanol, 1% formic acid, then vortexed and centrifuged at 14300 rpm (16457 g) at 4° C. for 20 minutes. The supernatant was transferred to a 96 well plate, then plates were incubated with 0.4% BSA, 37° C. for ½ hour. Injection volume was 25 μl.

For sample clean up a TurboFlow C8 (0.5×50 mm) or a Cyclone column (0.5×50 mm) both from Thermo Scientific, Franklin, Mass., USA, was used and the LC separation was done either on a Proteo 4 μm column (2.0×50 mm) or a Onyx C18 column (2.0×50 mm) both from Phenomenex, Torrance, Calif., USA. Eluents were isocratic and gradient combinations of methanol, acetonitril, Milli-Q water and formic acid. Selective detection was done by mass spectrometry operated in positive mode ionisation.

Non-compartmental analysis (NCA): Plasma concentration-time profiles are analyzed by non-compartmental pharmacokinetics analysis (NCA) using WinNonlin Professional 5.0 (Pharsight Inc., Mountain View, Calif., USA). NCA is performed using the individual plasma concentration-time profiles from each animal.

### Assay (VI): Assay for Determining Effect on Acute Food Intake

Mean cumulative food intake after single dosing in lean mice was determined in BioDaq system for automatic monitoring of food intake. 32 lean C57BI6J mice, 8 weeks old, were used in the study. The mice were housed two per cage but with a dividing wall containing holes to separate the mice. They were housed in reversed daily rhythm and with ad libitum access to food and water. The mice were acclimatized to the system for two weeks before start of the study. The system consisted of 32 individual boxes with a scale connected to each box which automatically registered the food available to the mice. Each time the mouse ate the weight reduction of food was registered and data collected on a computer. Data was collected continuously. The collected data was analysed using GraphPadPrism software.

Before start of the study the mice were fasted for approx. 18 hours. The mice were treated s.c. (10 ml/kg) with analogues dissolved in 50 mM Na₂HPO₄, 0.145M NaCl, 0.05% Tween 80, pH 7.4. Treatment took place 30 minutes before lights go out. Data was collected up to 48 hours after dosing.

Assay (VII): Determination of Effect on Body Weight and/or Body Composition

Additional assays useful to the invention include those that can determine the effect of PYY analogues or derivatives thereof on body weight and/or body composition. An exemplary assay is the following which involves utilization of an ob/ob mouse model for metabolic disease: ob/ob mice (Taconic, Hudson, N.Y.) on reversed day/night cycle and with access to a regular diet (Altromin 1324, Brogaarden, Denmark) are used. The mice are weighed on a weekly basis. Mice are used in the study when they have reached a body weight of at least 40 gram. Before starting the study all mice are scanned for body composition (NMR scan). One week before starting the study the mice are weighed daily to get a stable baseline and to acclimatize them to the procedure. The mice are divided into one group (n=10) receiving s.c. dosing of vehicle and groups of n=10 animals receiving PYY analogues or derivatives thereof by s.c. dosing of different doses or dosing intervals. At least one PYY analogue or derivative thereof and optionally at least one control compound, such as human PYY(1-36) or human PYY(3-36), are dissolved in 50 mM NaH₂PO₄, 165 mM NaCl, pH=7.4. Dosing is performed once daily at the same time point every day, shortly before lights off. As an alternative to s.c. administration some or all of the PYY analogues or derivatives thereof can be delivered via Alzet osmotic minipumps. The pumps can be set to deliver any amount of the PYY analogues or derivatives thereof, e.g., 1 μmol/kg/24 hours. The mice are dosed for 3 weeks. Body weight for all mice is recorded daily in combination with dosing. After 1 week and 3 weeks of treatment, the mice are scanned for body composition using a QNMR system (Echo Medical Systems, Houston, Tex.). Thereafter the mice are euthanized with cervical dislocation. Data are analysed in Graph Pad Prism. Statistical significance is assessed by comparing the groups with ANOVA followed by Tukey's post-hoc test. A p-value <0.05 is considered statistically significant.

Respiratory quotient (RQ, defined as CO₂ production divided by O₂ consumption) and metabolic rate can be determined using whole-animal indirect calorimetry (Oxymax, Columbus Instruments, Columbus, Ohio). The mice can be euthanized by isoflurane overdose, and an index of adiposity (bilateral epididymal fat pad weight) measured. In the methods of the invention the PYY analogue or derivative thereof are those having potency in one of the assays described herein, such as the assay to determine food intake, gastric emptying, pancreatic secretion, weight reduction or body composition assays.

### Assay (VIII): Measurement of Gastric Emptying

An exemplary assay for measurement of gastric emptying is described in the materials and methods section page 1326 under the headline “Gastric emptying” in Asakawa A et al., Characterization of the effects of pancreatic polypeptide in the regulation of energy balance, Gastroenterology, 2003, 124, 1325-1336.

### Assay (IX): Measurement of Appetite

Appetite can be measured by any means known to one of skill in the art. For example, in humans, decreased appetite can be assessed by a psychological assessment. In such an embodiment, administration of the receptor agonist results in a change in perceived hunger, satiety, and/or fullness. Hunger can be assessed by any means known to one of skill in the art. In one embodiment hunger is assessed using psychological assays, such as by an assessment of hunger feelings and sensory perception using e.g. a questionnaire.

### Example 2

A comparison of in vitro half lifes determined in minipig plasma (Assay (IV)) and Y2, Y1 and Y5 receptor potencies (Assay (I), (II) and (III), respectively) of PYY(3-36) analogues is shown in Table 2. This experiment shows that introduction of certain modifications into PYY prolongs the half life of PYY in vitro.

TABLE 2
In vitro half life (t½) in minipig plasma and Y2, Y1 and Y5
receptor potencies of PYY(3-36) compounds (Y2R, Y1R and
Y5R is the Y2, Y1 and Y5 receptor, respectively)

| | | In vitro t½ | Y2R EC50, | Y1R EC50, | Y5R EC50, |
|---|---|---|---|---|---|
| SEQ | | Assay (IV) | Assay (I) | Assay (II) | Assay (III) |
| ID NO | PYY(3-36) compound | (min) | (nM) | (nM) | (nM) |
| 1 | PYY(3-36) | 70 | 0.77 | 20 | 8.6 |
| 11 | [Ca-methyl | 84 | 40 | >1000 | n/a |
| | Phe36] PYY(3-36) | | | | |
| 12 | [N-methyl Gln34] | 406 | 1.7 | 0.24 | n/a |
| | PYY(3-36) | | | | |
| 13 | [β-homo Gln34] | 279 | 97 | 65 | n/a |
| | PYY(3-36) | | | | |
| 14 | [N-methyl Arg35] | 436 | 4.0 | >1000 | 860 |
| | PYY(3-36) | | | | |
| 15 | [N-methyl Arg33] | 59 | 69 | >1000 | n/a |
| | PYY(3-36) | | | | |
| 16 | [Agp35] | 231 | 320 | 740 | >1000 |
| | PYY(3-36) | | | | |
| 17 | [Agb35] | 195 | 75 | 440 | 300 |
| | PYY(3-36) | | | | |
| 18 | [hArg35] | 128 | 79 | 520 | >1000 |
| | PYY(3-36) | | | | |
| 19 | [N-methyl Thr32] | n/a | 990 | >1000 | >1000 |
| | PYY(3-36) | | | | |

n/a: not analysed

### Example 3

Y2, Y1 and Y5 receptor potencies (Assay (I), (II) and (III), respectively) were determined for derivatives of PYY analogues and human PYY(3-36) and the results are shown in Table 3.

TABLE 3
Y2, Y1 and Y5 receptor potencies of PYY(3-36) compounds (Y2R, Y1R and Y5R
is the Y2, Y1 and Y5 receptor, respectively).

| | | Y2R EC50, | Y1R EC50, | Y5R EC50, |
|---|---|---|---|---|
| SEQ | | Assay (I), | Assay (II), | Assay (III), |
| ID NO | PYY(3-36) compound | (nM) | (nM) | (nM) |
| 1 | PYY(3-36) | 0.77 | 20 | 8.6 |
| 2 | N-α*-PYY(3-36) | 4.6 | 13 | 2.4 |
| 3 | N-α*-[N-methyl Gln34]-PYY(3-36) | 2.8 | 1.0 | n/a |
| 4 | N-α*-[N-methyl Arg35]-PYY(3-36) | 7.9 | 290 | 230 |
| 6 | N-α*-[Aib25,N-methyl Gln34]- PYY(3-36) | 100 | 2.2 | n/a |
| 8 | N-α^{#}-[Ala20]-PYY(3-36) | 17 | 680 | 180 |
| 9 | N-α^{#}-[N-methyl Arg35]-PYY(3-36) | 31 | 7.5 | 83 |
| 10 | N-α^{#}-[Ala27]-PYY(3-36) | 39 | 200 | 70 |
| 20 | N-α^{#}-[Agp35]-PYY(3-36) | 370 | 710 | >1000 |
| 21 | N-α^{#}-[Agb35]-PYY(3-36) | 220 | 73 | >1000 |
| 22 | N-α^{#}-[Homo-Arg35]-PYY(3-36) | 385 | 670 | >1000 |
| 23 | [NArg35]-PYY(3-36) | 330 | 447 | >1000 |
| 24 | [NGln34]-PYY(3-36) | 394 | 419 | 30 |
| 25 | N-α^{#}-[NArg35]PYY(3-36) | 627 | 362 | >1000 |
| 26 | N-α^{#}-[NGln34]-PYY(3-36) | 450 | 370 | 52 |
| 27 | [N-methyl Tyr36]-PYY(3-36) | 15 | >1000 | 64 |
| 28 | [Ca-methyl Tyr36]-PYY(3-36) | 74 | 760 | >1000 |
| 29 | N-ε¹³*[Lys13, N-methyl Tyr36]-PYY(3-36) | 710 | >1000 | n/a |
| 30 | N-α^{#}[Ca-methyl Tyr36]-PYY(3-36) | 280 | n/a | n/a |
| 31 | N-α^{#}-PYY(3-36) | 12 | 27 | 20 |
| 32 | N-α-acetyl-[N-methyl Arg35]-PYY(3-36) | 6.5 | 64 | 190 |
| 33 | N-α^{#}-[NTyr36]-PYY(3-36) | 396 | 442 | >1000 |
| 34 | [NTyr36]-PYY(3-36) | 336 | 521 | >1000 |
| 35 | N-α-acetyl-N-ε^{10#}-[Lys10, | 17 | 426 | 191 |
| | N-methyl Arg35]-PYY(3-36) | | | |

*= [2-(2-{2-[2-(2-{2-[(S)-4-Carboxy-4-({trans-4-[(19-carboxynondecanoylamino)methyl]cyclohexanecarbonyl}
amino)-butyrylamino]-ethoxy}ethoxy)acetylamino]ethoxy}ethoxy)acetyl];
^{#}= {2-(2-{2-[2-(2-{2-[4-Carboxy-4-(17-carboxyheptadecanoylamino)butyrylamino]-ethoxy}ethoxy)-acetylamino]-
ethoxy}ethoxy)acetyl};
n/a: not analysed

### Example 4

Half-life of PYY analogous or derivatives thereof and hPYY(3-36) were determined according to Assay (V), PK i.v. minipig, as described herein. The results are shown in Table 4. This experiment shows that introduction of certain modifications into PYY prolongs the half life of PYY in vivo.

TABLE 4
In vivo Half-life (t½) of derivatives of PYY(3-36)
compounds determined in minipigs

| SEQ ID | | | | Dose | t½ |
|---|---|---|---|---|---|
| NO | PYY(3-36) compound | RoA¹ | n | (nmol/kg) | (h) |
| 1 | PYY(3-36) | i.v | 3 | 47 | 0.2 |
| 2 | N-α*-PYY(3-36) | i.v. | 3 | 9.5 | 8 |
| | | i.v. | 2 | 50 | 25 |
| | | s.c. | 4 | 29.7 | 30 |
| 3 | N-α*-[N-methyl Gln34]-PYY(3-36) | i.v. | 4 | 4 | 33 |
| 4 | N-α*-[N-methyl Arg35]-PYY(3-36) | i.v. | 3 | 30 | 73 |
| | | i.v. | 4 | 52 | 104 |
| | | s.c. | 4 | 57 | 93 |
| 6 | N-α*[Aib25,N-methyl Gln34] | i.v. | 4 | 10.4 | 46 |
| | PYY(3-36) | | | | |
| 9 | N-α^{#}-[N-methyl Arg35]-PYY(3-36) | i.v. | 3 | 10 | 80 |
| 11 | [Cα-methyl Phe36]PYY(3-36) | i.v. | 2 | 3.7 | 0.1 |
| | | i.v. | 2 | 48 | 1.0 |
| 27 | [N-methyl Tyr36]PYY(3-36) | i.v. | 3 | 4.8 | 0.3 |
| 29 | N-ε^{13#}-[Lys13, N-methyl Tyr36]] | i.v. | 4 | 1.7 | 20 |
| | PYY(3-36) | | | | |
| 31 | N-α^{#}-PYY(3-36) | i.v. | 2 | 50 | 17 |

¹) RoA: route of administration.
*= [2-(2-{2-[2-(2-{2-[(S)-4-Carboxy-4-({trans-4-[(19-carboxynondecanoylamino)methyl]cyclohexanecarbonyl}amino)-butyrylamino]-ethoxy}ethoxy)acetylamino]ethoxy}acetyl]
^{#}= {2-(2-{2-[2-(2-{2-[4-Carboxy-4-(17-carboxyheptadecanoylamino)butyrylamino]-
ethoxy}ethoxy)-acetylamino]-ethoxy}ethoxy)acetyl}

### Example 5

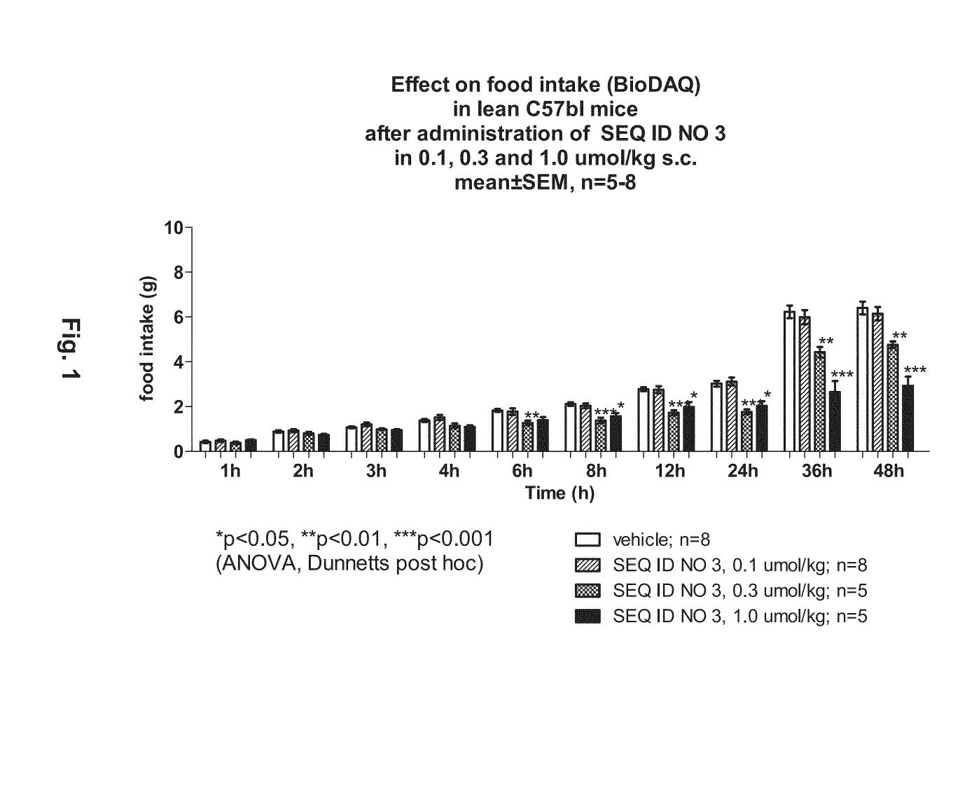

The effect of PYY derivatives on food intake was determined in lean fasted-re-fed C57BL/6 mice according to Assay (VI). The mice were administrated s.c. doses of the peptides in 0.1, 0.3 and 1.0 μmol/kg as well as vehicle alone 30 min before food return and cumulative food intake was measured over 48 hr. The results are shown in Table 5 and FIG. 1 for SEQ ID NO: 3.

TABLE 5
Mean cumulative food intake (n = 5-8 per group)

| Time after | Mean cumulative food intake [g] | | | |
|---|---|---|---|---|
| injection | | SEQ ID 3 | SEQ ID 3 | SEQ ID 3 |
| (h) | vehicle | 0.1 μmol/kg | 0.3 μmol/kg | 1 μmol/kg |
| 1 | 0.43 | 0.48 | 0.39 | 0.51 |
| 2 | 0.89 | 0.93 | 0.80 | 0.74 |
| 3 | 1.08 | 1.21 | 0.98 | 0.95 |
| 4 | 1.38 | 1.51 | 1.15 | 1.09 |
| 6 | 1.83 | 1.78 | 1.27** | 1.40 |
| 8 | 2.12 | 2.04 | 1.38*** | 1.57* |
| 12 | 2.78 | 2.75 | 1.73*** | 1.99* |
| 24 | 3.02 | 3.12 | 1.76*** | 2.03* |
| 36 | 6.23 | 5.99 | 4.43** | 2.66*** |
| 48 | 6.40 | 6.14 | 4.76** | 2.93*** |

*p < 0.05,
**p < 0.01,
***p < 0.001 (ANOVA, Dunnetts post hoc)

### Example 6

The effect of PYY derivatives on the mean cumulative food intake was determined according to Assay (VI). The results are shown in Table 6-8.

TABLE 6
Mean cumulative food intake (n = 7-8 per group)

| Time after | Mean cumulative food intake [g] | | | |
|---|---|---|---|---|
| injection | | SEQ ID 2 | SEQ ID 2 | SEQ ID 2 |
| (h) | vehicle | 0.1 μmol/kg | 0.3 μmol/kg | 1 μmol/kg |
| 1 | 0.49 | 0.48 | 0.58 | 0.43 |
| 2 | 0.91 | 0.95 | 0.92 | 0.80 |
| 3 | 1.16 | 1.25 | 1.18 | 1.10 |
| 4 | 1.64 | 1.58 | 1.57 | 1.43 |
| 6 | 2.22 | 1.96 | 1.88 | 1.57** |
| 8 | 2.48 | 2.28 | 2.21 | 1.87 |
| 12 | 3.32 | 2.93 | 2.88 | 2.26** |
| 24 | 3.55 | 3.24 | 2.92 | 2.28*** |
| 36 | 7.37 | 7.27 | 6.24* | 2.98*** |
| 48 | 7.61 | 7.53 | 6.52 | 3.33*** |

*p < 0.05,
**p < 0.01,
***p < 0.001 (ANOVA, Dunnetts post hoc)

TABLE 7
Mean cumulative food intake (n = 6-8 per group)

| Time after | Mean cumulative food intake [g] | | | |
|---|---|---|---|---|
| injection | | SEQ ID NO: 4 | SEQ ID NO: 4 | SEQ ID NO: 4 |
| (h) | vehicle | 0.1 μmol/kg | 0.3 μmol/kg | 1.0 μmol/kg |
| 1 | 0.49 | 0.46 | 0.51 | 0.34 |
| 2 | 0.94 | 0.81 | 0.86 | 0.70 |
| 3 | 1.40 | 1.13 | 1.15 | 0.96* |
| 4 | 1.74 | 1.39 | 1.52 | 1.17** |
| 6 | 2.41 | 1.89** | 1.80** | 1.43*** |
| 8 | 2.96 | 2.35*** | 2.24*** | 1.84*** |
| 12 | 3.78 | 3.08*** | 2.75*** | 2.52*** |
| 24 | 4.09 | 3.43** | 3.12*** | 3.24*** |
| 36 | 7.78 | 7.01 | 6.58** | 6.10*** |
| 48 | 7.98 | 7.39 | 6.82* | 6.54** |

*p < 0.05,
**p < 0.01,
***p < 0.001 (ANOVA, Dunnetts post hoc)

TABLE 8
Mean cumulative food intake (n = 7-8 per group)

| Time after | Mean cumulative food intake [g] | | |
|---|---|---|---|
| injection | | SEQ ID 31 | SEQ ID 9 |
| (h) | vehicle | 1 μmol/kg | 1 μmol/kg |
| 1 | 0.49 | 0.58 | 0.52 |
| 2 | 0.99 | 0.92 | 0.89 |
| 3 | 1.28 | 1.10 | 1.01 |
| 4 | 1.65 | 1.29 | 1.21* |
| 6 | 2.13 | 1.61* | 1.58* |
| 8 | 2.74 | 2.07* | 2.19* |
| 12 | 3.61 | 2.60** | 2.84* |
| 24 | 4.46 | 2.88*** | 3.08*** |
| 36 | 8.51 | 6.41** | 6.41** |
| 48 | 9.23 | 6.88** | 6.91** |

*p < 0.05,
**p < 0.01,
***p < 0.001 (ANOVA, Dunnetts post hoc)

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

1. A PYY analogue or a derivative thereof comprising
i. at least one serum albumin binding side chain comprising an alkyl chain with at least 14 carbon atoms, wherein said alkyl chain optionally further comprises a distal carboxylic acid or a distal tetrazole group; and
ii. at least one amino acid residue substituted into a proteinogenic or non-proteinogenic amino acid residue selected from the group consisting of 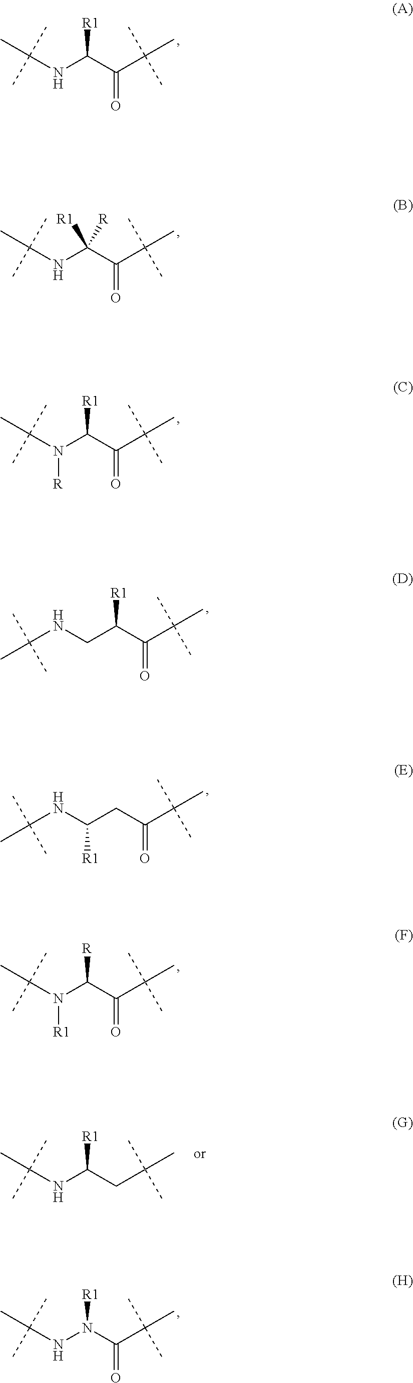
wherein R1 is side a chain of an amino acid and R is H or C1-C12 alkyl.

2. The PYY analogue or a derivative thereof according to claim 1, wherein said proteinogenic or non-proteinogenic amino acid residue is in at least one position selected from the group consisting of position 36, 35, 34, 33, 32, 31, 30, 29, 28, 27, 26, 25, 21, 20, 19 and 4 or from the group consisting of position 33, 34, 35 and 36.

3. The PYY analogue or a derivative thereof according to claim 1, wherein said scrum albumin binding side chain is selected from the group consisting of A-B-C-D-, A-C-D-, A-B-C- and A-C-, wherein A- is 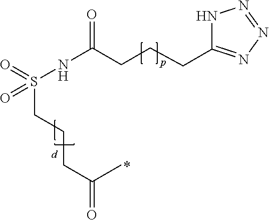
wherein p is selected from the group consisting of 10, 11, 12, 13 and 14, and d is selected from the group consisting of 0, 1, 2, 3, 4 and 5, and
-B- is selected from the group consisting of 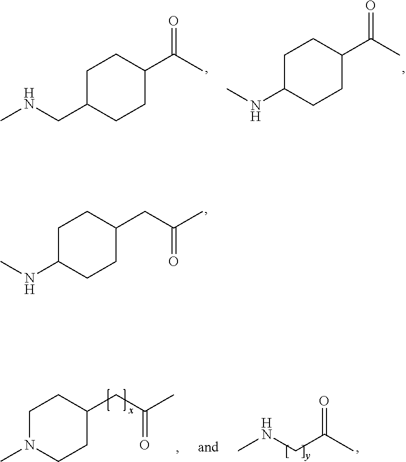
wherein x is selected from the group consisting of 0, 1, 2, 3 and 4, and y is selected from the group consisting of 1, 2, 3, 4, 5, 6, 7, 8, 9, 10, 11 and 12,
or A- is 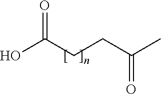
wherein n is selected from the group consisting of 12, 13, 14, 15, 16 17, 18 and 19, and B is selected from the group consisting of 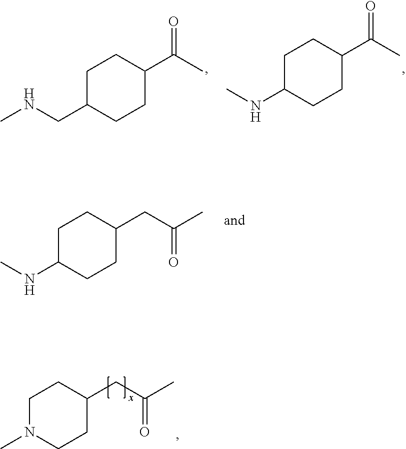
wherein x is selected from the group consisting of 0, 1, 2, 3 and 4, and
-C- is selected from the group consisting of 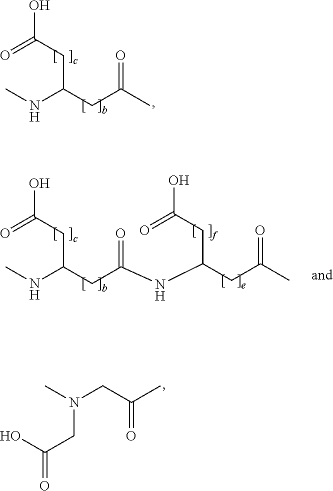
wherein b and e are each independently selected from the group consisting of 0, 1 and 2, and c and f are each independently selected from the group consisting of 0, 1 and 2 with the proviso that b is 1 or 2 when c is 0, or b is 0 when c is 1 or 2, and e is 1 or 2 when f is 0, or e is 0 when f is 1 or 2, and
-D- is attached to said amino acid residue and is a spacer, such as at least one 8-amino-3,6-dioxaoctanoic acid (Oeg) molecule.

4. The PYY analogue or a derivative thereof according to claim 1, wherein said serum albumin binding side chain is
2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl or
2-(2-{2-[2-(2-{2-[4-Carboxy-4-(17-carboxyheptadecanoylamino)butyrylamino]ethoxy}-ethoxy)acetylamino]-ethoxy}ethoxy)acetyl.

5. The PYY analogue or a derivative thereof according to claim 1, wherein said analogue or derivative comprises the amino acid sequence of the formula (I):
Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅-Xaa₂₆-Xaa₂₇-Xaa₂₈-Xaa₂₉-Xaa₃₀-Xaa₃₁-Xaa₃₂-Xaa₃₃-Xaa₃₄-Xaa₃₅-Xaa₃₆ Formula (I)
wherein
Xaa₁ is Tyr, Phe, Ala, 2,3-diaminopropionic acid, 2,4-diaminobutyric acid, ornithine, Lys or absent;
Xaa₂ is Pro, Ala, Len, Phe, hydroxyproline, 2,3-diaminopropionic acid, 2,4-diaminobutyric acid, ornithine, Lys or absent;
Xaa₃ is Ile, Val, Leu (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₄ is Lys, Gln, 2,3-diaminopropionic acid, 2,4-diaminobutyric acid, ornithine or Lys;
Xaa₅ is Pro, 2,3-diaminopropionic acid, 2,4-diaminobutyric acid, ornithine or Lys;
Xaa₆ is Glu, 2,3-diaminopropionic acid, 2,4-diaminobutyric acid, ornithine or Lys;
Xaa₇ is Ala, 2,3-diaminopropionic acid, 2,4-diaminobutyric acid, ornithine or Lys;
Xaa₈ is Pro, Ala, 2,3-diaminopropionic acid, 2,4-diaminobutyric acid, ornithine or Lys;
Xaa₉ is Gly, 2,3-diaminopropionic acid, 2,4-diaminobutyric acid, ornithine or Lys;
Xaa₁₀ is Glu, 2,3-diaminopropionic acid, 2,4-diaminobutyric acid, ornithine or Lys;
Xaa₁₁ is Asp, Ala, 2,3-diaminopropionic acid, 2,4-diaminobutyric acid, ornithine or Lys;
Xaa₁₂ is Ala, 2,3-diaminopropionic acid, 2,4-diaminobutyric acid, ornithine or Lys;
Xaa₁₃ is Ser, 2,3-diaminopropionic acid, 2,4-diaminobutyric acid, ornithine or Lys;
Xaa₁₄ is Pro or hydroxyproline;
Xaa₁₅ is Glu, 2,3-diaminopropionic acid, 2,4-diaminobutyric acid, ornithine or Lys;
Xaa₁₆ is Glu, 2,3-diaminopropionic acid, 2,4-diaminobutyric acid, ornithine or Lys;
Xaa₁₇ is Leu, Val, Ile, homoleucine, norleucine, (1-aminocyclopentyl) carboxylic acid, (1-aminocyclohexyl) carboxylic acid or 1-aminobutyric acid;
Xaa₈ is Asn, 2,3-diaminopropionic acid, 2,4-diaminobutyric acid, ornithine or Lys;
Xaa₁₉ is Arg, Ala, 2,3-diaminopropionic acid, 2,4-diaminobutyric acid, ornithine or Lys;
Xaa₂₀ is Tyr, Ala, Phe, 3-pyridylalaine, 2,3-diaminopropionic acid, 2,4-diaminobutyric acid, ornithine or Lys;
Xaa₂₁ is Tyr, Ala, Phe, 2,3-diaminopropionic acid, 2,4-diaminobutyric acid, ornithine or Lys;
Xaa₂₂ is Ala, 2,3-diaminopropionic acid, 2,4-diaminobutyric acid, ornithine or Lys;
Xaa₂₃ is Ser, 2,3-diaminopropionic acid, 2,4-diaminobutyric acid, ornithine or Lys;
Xaa₂₄ is Leu, Ile, Val, homoleucine, norleucine, (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₂₅ is Arg, Ala, His, Tyr, aminoisobutyric acid, 2,3-diaminopropionic acid, 2,4-diaminobutyric acid, ornithine or Lys;
Xaa₂₆ is His, Arg, 2,3-diaminopropionic acid, 2,4-diaminobutyric acid, ornithine or Lys;
Xaa₂₇ is Tyr, Ala, Phe, homoPhe or 3-pyridylalanine, 2,3-diaminopropionic acid, 2,4-diaminobutyric acid, ornithine or Lys;
Xaa₂₈ is Ile, Val, Leu, homoleucine, norleucine, (1-aminocyclopentyl) carboxylic acid, (1-aminocyclohexyl) carboxylic acid, aminoisobutyric acid, 1-aminobutyric acid, 2,3-diaminopropionic acid, 2,4-diaminobutyric acid, ornithine or Lys;
Xaa₂₉ is Asn, Gln, 2,3-diaminopropionic acid, 2,4-diaminobutyric acid, ornithine or Lys;
Xaa₃₀ is Met, Leu, Val, Ile, homoleucine, aminoisobutyric acid, norleucine, (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₃₁ is Leu, Val, Ile, aminoisobutyric acid, homoleucine, norleucine, (1-aminocyclopentyl) carboxylic acid, (1-aminocyclohexyl) carboxylic acid, 1-aminobutyric acid, 2,3-diaminopropionic acid, 2,4-diaminobutyric acid, ornithine or Lys;
Xaa₃₂ is Ser, Thr, 2,3-diaminopropionic acid, 2,4-diaminobutyric acid, ornithine or Lys;
Xaa₃₃ is Arg, N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, Amino-3-(4-guanidino-phenyl)-propionic acid or Amino-(1-carbamimidoyl-piperidin-4-yl)acetic acid;
Xaa₃₄ is Gln, Asn, His, Pro, N-methyl Gln, β-homo Gln, (2-Carbamoyl-ethylamino)-acetic acid, N-methyl Asn or N-methyl His;
Xaa₃₅ is Arg, N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid, or Amino-(1-carbamimidoyl-piperidin-4-yl)-acetic acid;
Xaa₃₆ is Tyr, Phe, N-methyl Tyr, C-α-methyl Phe, 3-pyridylalanine or (4-Hydroxybenzylamino)-acetic acid;
wherein at least one of Xaa₃₃, Xaa₃₄, Xaa₃₅ and Xaa₃₆ is selected from the group consisting of
Xaa₃₃ is N-methyl Arg, methyllysine, dimethyllysine, trimethyllysine, 2-amino-3-guanidinopropionic acid, 2-amino-4-guanidino-butyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrolidin-2-yl)propionic acid, 2-Amino-4-(2-amino-pyrimidin-4-D-butyric acid, 2-Amino-3-(4-guanidino-phenyl)-propionic acid or Amino-(1-carbamimidoyl-piperidin-4-yl)-acetic acid;
Xaa₃₄ is N-methyl Gln, β-homo Gln, (2-Carbamoyl-ethylamino)-acetic acid, N-methyl Asn or N-methyl His;
Xaa₃₅ is N-methyl Arg, methyllysine, dimethyllysine, trimethyllysine, 2-amino-3-guanidinopropionic acid, 2-amino-4-guanidino-butyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrolidin-2-yl)propionic acid, 2-Amino-4-(2-amino-pyrimidin-4-yl)-butric acid, 2-Amino-3-(4-guanidino-phenyl)-propionic acid, or Amino-(1-carbamimidoyl-piperidin-4-yl)-acetic acid; and
Xaa₃₆ is N-met Tyr, C-α-methyl Phe, 3-pyridylalanine or (4-Hydroxy-benzylamino)acetic acid.

6. The PYY analogue or a derivative thereof according to claim 1, wherein Xaa₃₃ is N-methyl Arg, methyllysine, dimethyllysine, trimethyllysine, 2-amino-3-guanidino-propionic acid, 2-amino-4-guanidinobutyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-1-carbamimidoyl-pyrrolidin-2-yl)-propionic acid, 2-Amino-4-(2-amino-pyrimidin-4-yl)butyric acid, 2-Amino-3-(4-guanidino-phenyl)-propionic acid, or Amino-(1-carbamimidoyl-piperidin-4-yl)-acetic acid.

7. The PYY analogue or a derivative thereof according to claim 1, wherein Xaa₃₄ is Pro, N-methyl Gln, β-homo Gln, (2-Carbamoyl-ethylamino)-acetic acid, N-methyl Asn or N-methyl His.

8. The PYY analogue or a derivative thereof according to claim 1, wherein Xaa₃₅ is N-methyl Arg, methyllysine, dimethyllysine, trimethyllysine, 2-amino-3-guanidino-propionic acid, 2-amino-4-guanidinobutyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butric acid, 2-Amino-3-(4-guanidino-phenyl)-propionic acid, or Amino-(1-carbamimidoyl-piperidin-4-yl)-acetic acid.

9. The PYY analogue or a derivative thereof according to claim 1 wherein Xaa₃₆ is N-methyl Tyr, C-alpha-methyl Phe, 3-pyridylalanine or (4-Hydroxy-benzylamino)-acetic acid.

10. The PYY analogue or a derivative thereof according to claim 1, wherein Xaa₁ and Xaa₂ are absent or Xaa₁, Xaa₂, Xaa₃ and Xaa₄ are absent.

11. The PYY analogue or a derivative thereof according to claim 1, wherein said analogue or derivative is selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 20, SEQ ID NO: 21, SEQ ID NO: 22, SEQ ID NO: 25, SEQ ID NO: 26, SEQ ID NO: 29, SEQ ID NO: 30, SEQ ID NO: 33 and SEQ ID NO: 35.

12. The PYY analogue or a derivative thereof according to claim 1, wherein said analogue or derivative has improved stability against C-terminal proteolytic breakdown which breakdown decreases functionality of said analogue or derivative as compared to human PYY(1-36) or human PYY(3-36), wherein said functionality is determined by Assay (IV), Assay (I) or Assay (V) as described herein.

13. The PYY analogue or a derivative thereof according to claim 1, wherein said analogue or a derivative does not comprise a derivatisation group and wherein said analogue or a derivative is not
[N-methyl Arg33] PYY(3-36),
[N-methyl Gln34] PYY(3-36),
[N-methyl Arg35] PYY(3-36),
[N-methyl Tyr36] PYY(1-36), or
[N-methyl Tyr36] PYY(3-36).

14. The PYY analogue or a derivative thereof according to claim 1, wherein said analogue or derivative is selected from the group consisting of SEQ ID NO: 11, SEQ ID NO: 13, SEQ ID NO: 16, SEQ ID NO: 17, SEQ ID NO: 18, SEQ ID NO: 19, SEQ ID NO: 23, SEQ ID NO: 24, SEQ ID NO: 28, SEQ ID NO: 32 and SEQ ID NO: 34.

15. (canceled)

16. A pharmaceutical composition comprising the PYY analogue or a derivative thereof according to claim 1 and at least one pharmaceutical excipient.

17. A method for treating diabetes, a condition responsive to Y receptor modulation, obesity, obesity-related diseases, and reduction of food intake comprising administering to a subject in need of such treatment a pharmaceutically effective amount of a PYY analogue or a derivative thereof according to claim 1.
